SEQUENCE LISTING (17 sequences)

SEQ ID NO: 1 - DNA, 1192 bp, Rattus sp.
atgcggccggccgcagagctgggccagacgctgagcagggccgggctctgccgacccctttgcctcctgctctgcgcttcgcagctaccgcacacgatgcacccccaaggccgcgccgcctccccacagctgctgctcggcctcttccttgtgctactgctgcttctgcagctgtccgcgccgtccagcgcctctgagaatcccaaggtgaagcaaaaagcgctgatccggcagagggaagtggtagacctgtataatgggatgtgcctacaaggaccagcaggagttcctggtcgcgatgggagccctggggccaatggcattcctggcacaccgggaatcccaggtcgggatggattcaaaggagagaaaggggagtgcttaagggaaagctttgaggaatcctggaccccaaactacaagcagtgttcatggagttcacttaattatggcatagatcttgggaaaattgcggaatgtacattcacaaagatgcgatccaacagcgctcttcgagttctgttcagtggctcgcttcggctcaaatgcaggaatgcttgctgtcaacgctggtattttacctttaatggagctgaatgttcaggacctcttcccattgaagctatcatctatctggaccaaggaagccctgagttaaattcaactattaatattcatcgtacttcctccgtggaaggactctgtgaagggattggtgctggactggtagacgtggccatctgggtcggcacctgttcagattaccccaaaggagacgcttctactgggtggaattctgtgtcccgcatcatcattgaagaactaccaaaataaagcccctgaaggtttcattccctgcctcatttacttgttaaatcaagcctctggatgggtcatttaaatgacatttcagaagtcacttatgtgctcagccaaatgaaaaagcaaagttaaatacgtttacagaccaaagtgtgatctcacactttaagatctagcattatccattttatttcaaccaaagatggtttcaggattttatttctcattgattactttttgagcctatataccggaatgctgttatagtctttaatatttcctactgttgacattttgaaacatataaaagttatgtctttgtaagagctgtatagaattattttatatgttaaataaatgcttcaaacaa

SEQ ID NO: 2 - protein, 245 aa, Rattus sp.
MHPQGRAASPQLLLGLFLVLLLLLQLSAPSSASENPKVKQKALIRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPELNSTINIHRTSSVEGLCEGIGAGLVDVAIWVGTCSDYPKGDASTGWNSVSRIIIEELPK

SEQ ID NO: 3 - DNA, 1220 bp, Homo sapiens
acgagggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatatttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaa

SEQ ID NO: 4 - protein, 243 aa, Homo sapiens
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 5 - protein, 277 aa, Rattus sp.
MRPAAELGQTLSRAGLCRPLCLLLCASQLPHTMHPQGRAASPQLLLGLFLVLLLLLQLSAPSSASENPKVKQKALIRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPELNSTINIHRTSSVEGLCEGIGAGLVDVAIWVGTCSDYPKGDASTGWNSVSRIIIEELPK

SEQ ID NO: 6 - RNA, 403 nt, Artificial sequence
  Description of Artificial SequenceREMODEL antisense ribonucleoprobe
ccacccaguagaagcgucuccuuugggguaaucugaacaggugccgacccagauggccacgucuaccaguccagcaccaaucccuucacagaguccuuccacggaggaaguacgaugaauauuaauaguugaauuuaacucagggcuuccuugguccagauagaugauagcuucaaugggaagagguccugaacauucagcuccauuaaagguaaaauaccagcguugacagcaagcauuccugcauuugagccgaagcgagccacugaacagaacucgaagagcgcuguuggaucgcaucuuugugaauguacauuccgcaauuuucccaagaucuaugccauaauuaagugaacuccaugaacacugcuuguaguuugggguccaggauuccucaaagcuu

SEQ ID NO: 7 - protein, 15 aa, Artificial sequence
  Description of Artificial Sequencecarboxy- terminal amino acids of REMODEL
GWNSVSRIIIEELPK

SEQ ID NO: 8 - protein, 24 aa, Artificial sequence
  Description of Artificial Sequenceinsulin signal peptide
MALLVHFLPLLALLALWEPKPTQA

SEQ ID NO: 9 - DNA, 734 bp, Artificial sequence
  Description of Artificial Sequencemyc-tagged REMODEL construct
atggcccccaaggccgcgccgcctccccacagctgctgctcggcctcttccttgtgctactgctgcttctgcagctgtccgcgccgtccagcgcctctgagaatcccaaggtgaagcaaaaagcgctgatccggcagagggaagtggtagacctgtataatgggatgtgcctacaaggaccagcaggagttcctggtcgcgatgggagccctggggccaatggcattcctggcacaccgggaatcccaggtcgggatggattcaaaggagagaaaggggagtgcttaagggaaagctttgaggaatcctggaccccaaactacaagcagtgttcatggagttcacttaattatggcatagatcttgggaaaattgcggaatgtacattcacaaagatgcgatccaacagcgctcttcgagttctgttcagtggctcgcttcggctcaaatgcaggaatgcttgctgtcaacgctggtattttacctttaatggagctgaatgttcaggacctcttcccattgaagctatcatctatctggaccaaggaagccctgagttaaattcaactattaatattcatcgtacttcctccgtggaaggactctgtgaagggattggtgctggactggtagacgtggccatctgggtcggcacctgttcagattaccccaaaggagacgcttctactgggtggaattctgtgtcccgcatcatcattgaagaactaccaaaa

SEQ ID NO: 10 - DNA, 513 bp, Homo sapiens
  misc_feature: longer cleavage fragment of human CTHRC1
attccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataa

SEQ ID NO: 11 - protein, 170 aa, Homo sapiens
  MISC_FEATURE: longer cleavage fragment of human CTHRC1
IPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 12 - DNA, 495 bp, Homo sapiens
  misc_feature: shorter cleavage fragment of human CTHRC1
atcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataa

SEQ ID NO: 13 - protein, 164 aa, Homo sapiens
  MISC_FEATURE: shorter cleavage fragment of human CTHRC1
IPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 14 - protein, 34 aa, Artificial sequence
  collagen domain of CTHRC1
GPAGVPGRDGSPGANGIPGTPGIPGRDGFKGEKG

SEQ ID NO: 15 - DNA, 102 bp, Artificial sequence
  collagen domain of CTHRC1
ggaccagcaggagttcctggtcgcgatgggagccctggggccaatggcattcctggcacaccgggaatcccaggtcgggatggattcaaaggagagaaaggg

SEQ ID NO: 16 - protein, 34 aa, Artificial sequence
  mouse collagen 1 alpha1
GSPGTAGARGNDGAVGAAGPPGPTGPTGPPGFGF

SEQ ID NO: 17 - DNA, 102 bp, Artificial sequence
  mouse collagen 1 alpha1
ggatcccctggcactgctggtgctcgcggtaacgatggtgctgttggtgctgctggaccccctggtcccaccggccccactggccctcctggcttcggattc

(19) United States
(12) Patent Application Publication
(10) Pub. No.: US 2005/0147602 A1
(43) Pub. Date: Jul. 7, 2005

(54) Compositions, methods and kits relating to CTHRC1, a novel modulator of collagen matrix

(75) Inventor: Volkhard Lindner, South Portland, ME (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Maine Medical Center Research Institute

(21) Appl. No.: 10/939,233

(22) Filed: Sep. 10, 2004

Related U.S. Application Data
(63) Continuation-in-part of application No. 10/045,992, filed on Oct. 19, 2001.
Application No. 10/045,992 is a continuation-in-part of application No. 09/692,081, filed on Oct. 19, 2000, now Pat. No. 6,630,325.
(60) Provisional application No. 60/503,933, filed on Sep. 18, 2003.
(60) Provisional application No. 60/504,107, filed on Sep. 18, 2003.

(51) Int. Cl. A01K 67/00; C07H 21/04; A61K 39/395; A61K 39/00; C07K 14/785

(52) U.S. Cl. 424/130.1; 435/69.7; 435/320.1; 435/325; 530/356; 536/23.5; 530/388.1; 514/12; 424/192.1; 800/8

(57) ABSTRACT

The invention relates to a novel CTHRC1 nucleic acid and protein encoded thereby. Expression of CTHRC1 is induced by injury in, among others, arteries and skin, and CTHRC1 is expressed in bone, cartilage, kidney, lung and brain. CTHRC1 expression is associated with collagen matrix production, arterial remodeling, arterial restenosis, constrictive remodeling, vessel injury, ectopic ossification, fibrosis, and the like. CTHRC1 also plays a role in cell-cell and cell-matrix adhesion, cell-migration, and bone, cartilage, skin and brain development. CTHRC1 also regulates the level of BMPs, including BMP1 and BMP4, and the invention encompasses methods relating to affecting the level of BMPs by affecting the level of CTHRC1. In addition, the invention relates to modulation of the level of CTHRC1 to affect processes associated with fibrosis mediated by formation of collagen matrix. The invention further relates to methods of treating, preventing, and/or detecting these diseases, disorders or conditions, where the methods comprise modulating or detecting CTHRC1 expression and/or production of CTHRC1 polypeptide. The invention also relates to affecting CTHRC1 expression using cytokines.

### CROSS-REFERENCE TO RELATED APPLICATIONS

The present application is a continuation-in-part of U.S. patent application Ser. No. 10/045,992, filed on Oct. 19, 2001, which is a continuation-in-part of U.S. patent application Ser. No. 09/692,081, filed on Oct. 19, 2000, from which it is entitled priority under 35 U.S.C. § 120, and U.S. Provisional Patent Application No. 60/503,933, filed Sep. 18, 2003, and U.S. Provisional Patent Application No. 60/504,107, filed Sep. 18, 2003, from which it is entitled priority under 35 U.S.C. § 119(e), each of which is incorporated by reference herein as if set forth in its entirety.

### STATEMENT REGARDING FEDERALLY SUPPORTED RESEARCH AND DEVELOPMENT

This invention was supported in part by U.S. Government funds (National Institutes of Health grant Nos. HL69182 and DE13248), and the U.S. Government may therefore have certain rights in the invention.

### BACKGROUND OF THE INVENTION

The present invention relates to arterial remodeling, restenosis, fibrosis, and formation of bone and cartilage. Arterial stenosis with reduction in blood flow is a common problem in many vascular diseases. Several growth factors have been implicated in the mechanisms leading to vascular stenosis. For instance, fibroblast growth factor 2 (FGF-2) has been identified as an important factor in mediating proliferation of smooth muscle cells leading to intimal lesion formation. Furthermore, it has been demonstrated that arterial stenosis in response to angioplasty is largely due to constrictive remodeling as a result of adventitial fibrosis. As more fully set forth below, transforming growth factor beta (TGF-β) signaling has been demonstrated to play an important role in arterial stenosis in that, among other things, inhibition of TGF-β signaling using a soluble TGF-β receptor type II dramatically reduced lumen narrowing by decreasing constrictive remodeling and adventitial matrix deposition as well by decreasing neointima formation. These results indicate the crucial role of TGF-β signaling in arterial response to injury.

Vascular remodeling is a response of blood vessels to both physiological and pathological stimuli, leading to either vessel enlargement (positive remodeling) or shrinkage (constrictive remodeling). It has been demonstrated that neointimal proliferation or intimal mass following angioplasty shows little correlation with restenosis because of permanent changes in vascular geometry (Kakuta et al., 1994, Circulation 89:2809-2815; Nunes et al., 1995, Arterioscler. Thromb. Vasc. Biol. 15:156-165). Constrictive remodeling has been shown to account for most of the restenosis process (Mintz et al., 1993, Circulation 88:1-654), and is now generally considered the predominant cause of restenosis. A successful therapeutic approach to restenosis, therefore, would target constrictive vascular remodeling.

Several growth factors have been implicated in the mechanisms leading to vascular stenosis, such as fibroblast growth factor-2 (FGF-2) and transforming growth factor-β (TGF-β). Specifically, cellular responses involving TGF-β in the adventitia have gained increased attention for their potential involvement in adventitial remodeling (Wilcox et al., 1996, Int. J. Radiat. Oncol. Biol. Phys. 36:789-796; Wilcox and Scott, 1996, Int. J. Cardiol. 54S:S21-35; Shi et al., 1996, Circulation 93:340-348). There is evidence that proliferative events occurring in the adventitia contribute to vascular remodeling and restenosis in response to vascular injury (Wilcox et al., 1996, Int. J. Radiat. Oncol. Biol. Phys. 36:789-796; Wilcox et al., 1997, Ann. N.Y. Acad. Sci. 811:437-447; Scott et al., 1996, Circulation 93:2178-2187). There is now general agreement that TGF-β is a potential factor in the adventitial remodeling process (Shi et al., 1996, Arterioscler. Thromb. Vasc. Biol. 16:1298-1305).

Although it is known that the TGF-β family of cytokines can have a variety of effects on vascular cells, very little is known about the role of this family of cytokines in vascular remodeling. TGF-β affects many functions including proliferation of smooth muscle cells (SMC) (Halloran et al., 1995, Am. J. Surg. 170:193-197). It has been demonstrated that inhibition of SMC proliferation by TGF-β1 occurs via extension of the G2 phase of the cell cycle (Grainger et al., 1994, Biochem. J. 299:227-235). In contrast, it has also been shown that inhibition of SMC proliferation by TGF-β1 is due to arrest in the late G1 phase of the cell cycle (Reddy and Howe, 1993, J. Cell Physiol. 156:48-55). SMC derived from atherosclerotic lesions responded to TGF-β1 with an increase in proliferation, and lower levels of TGF-β receptor II (TGF-βRII) have been implicated in the lack of inhibition by TGF-β1 in these cells (McCaffrey et al., 1995, J. Clin. Invest. 96:2667-2675).

Further studies have established that TGF-β1 stimulates SMC proliferation in vitro. Low doses of TGF-β1 stimulated SMC proliferation via platelet-derived growth factor (PDGF)-amino acid (AA)-dependent and PDGF-AA-independent mechanisms, while higher doses of TGF-β1 were inhibitory (Battegay et al., 1990, Cell 63:515-524; Stouffer and Owens, 1994, J. Clin. Invest. 93:2048-2055). Bifunctional effects of TGF-β1 in migration assays with SMC were also demonstrated (Koyama et al., 1990, Biochem. Biophys. Res. Commun. 169:725-729; Mii et al., 1993, Surgery 114:464-470).

TGF-β1 also plays a role in intimal lesion formation as indicated by a 5-7 fold induction of TGF-β1 mRNA in the balloon-injured rat carotid artery, with elevated levels of TGF-β1 mRNA persisting for 2 weeks (Majesky et al., 1991, J. Clin. Invest. 88:904-910). During the 2 week period, elevated TGF-β1 mRNA levels correlated with increases in mRNA expression of fibronectin and alpha-2 (I) and alpha-1 (III) collagens. These studies also demonstrated that infusion of recombinant TGF-β1 caused an increase in intimal SMC proliferation in vivo (id.).

Among clinically significant findings regarding the role of TGF-β signaling in arterial response to injury, it has been demonstrated that TGF-β1 mRNA expression in restenotic lesions compared to primary atherosclerotic lesions is increased (Nikol et al., 1992, J. Clin. Invest. 90:1582-1592). In the rat balloon injury model, treatment with TGF-β1 antibodies caused a small but significant reduction in neointima formation (Wolf et al., 1994, J. Clin. Invest. 93:1172-1178). Overexpression of TGF-β1 in the rat carotid artery by adenoviral gene transfer led to transient neointima formation with cartilaginous metaplasia that almost completely resolved within 8 weeks (Shulick et al., 1998, Proc. Natl. Acad. Sci. USA 95:6983-6988). Without wishing to be bound by any particular theory, TGF-β1 may also affect vascular tone since the factor was found to suppress nitric oxide synthase expression (Perella et al., 1996, J. Biol. Chem. 271:13776-13780) while at the same time inducing the vasoconstrictor endothelin in SMC in vitro (Kurihara et al., 1989, Biochem. Biophys. Res. Commun. 159:1435-1440). Further, TGF-β1 has been implicated in anti-apoptotic effects in SMC (Herbert and Carmeliet, 1997, FEBS Lett. 413:401-404).

Studies examining the expression of TGF-β ligand and TGF-β receptor (TGF-βR) mRNAs using reverse transcriptase polymerase chain reaction (RT-PCR) analysis revealed that TGF-β1, TGF-β3, and TGF-βII mRNA levels were increased in the media of the injured rat carotid artery (Ward et al., 1997, Arterioscler. Thromb. Vasc. Biol. 17:2461-2470) and expression of TGF-β2 and TGF-β3 were also reported in SMC of the lung vasculature (Khalil et al., 1996, Am. J. Respir. Cell Mol. Biol. 14:131-138; Pelton et al., 1991, Am. J. Respir. Cell Mol. Biol. 5:522-530). However, reduced levels of TGF-βRII were demonstrated in human atherosclerotic lesions (McCaffrey et al., 1995, J. Clin. Invest. 96:2667-2675). The three TGF-β ligands have overlapping functions and all of them induce expression of the alpha-1 (I), alpha-2 (I) and alpha-1 (III) chains of collagen (Bray et al., 1998, Hypertension 31:986-994).

The role of TGF-β isoforms in vascular repair processes was examined using a rat balloon catheter denudation model (Smith et al., 1999, Circ. Res. 84:1212-1222). Proliferating and quiescent SMC in denuded vessels expressed high levels of mRNA for TGF-β1, TGF-β2, and TGF-β3, and lower levels of TGF-βRII mRNA (Smith et al., 1999, Circ. Res. 84:1212-1222). The role of TGF-β signaling in the rat carotid artery balloon injury model was tested and it was shown that control vessels developed an extensive neointima and adventitial fibrosis with abundant collagen production. Vessels from animals injected with a recombinant soluble TGF-βRII (designated as “TGF-βR:Fc”) revealed only little neointima formation and much less collagen deposition in the adventitia. The adventitia also contained significantly fewer cells, indicating that the proliferation of adventitial fibroblasts is mediated by TGF-β. Further, inhibition of TGF-β signaling with TGF-βR:Fc dramatically reduced lumen narrowing by decreasing constrictive remodeling and adventitial matrix deposition, as well as neointima formation.

TGF-β has been implicated in myofibroblastic transdifferentiation (Orlandi et al., 1994, Exp. Cell Res. 214:528-536; Desmouliere et al., 1993, J. Cell Biol. 122:103-111; Verbeek et al., 1994, Am. J. Pathol. 144:372-382), causing fibroblasts to transiently express smooth muscle α-actin (Darby et al., 1990, Lab. Invest. 63:21-29). The expression of smooth muscle α-actin in the carotid artery was examined using immunostaining at 4 days after balloon denudation when proliferation of adventitial fibroblasts is rapid. Immunoreactive smooth muscle α-actin was either completely absent or markedly reduced in the outer adventitia of vessels from rats treated with TGF-βR:Fc compared to controls. This result demonstrated that the induction of smooth muscle α-actin expression by adventitial fibroblasts is at least in part mediated by TGF-β.

Morphometric analysis of the carotid arteries demonstrated significant increases in lumen area in all rats treated with TGF-βR:Fc with an approximate 88% increase with a dose of 2 mg/kg given every other day for 2 weeks. Further, a dose of 0.5 mg/kg every other day for 2 weeks caused nearly a 60% increase in lumen area despite the fact that intimal lesion formation was not affected by this dose. These results indicate that loss of lumen area is in large part due to constrictive remodeling and measurements of the perimeter of the neointima (IEL) and media (EEL) demonstrated that all doses of TGF-βR:Fc used in this study significantly inhibited the reduction in IEL and EEL.

The effect of TGF-βR:Fc on remodeling is highly relevant to the clinical situation of restenosis after angioplasty (Mintz et al., 1993, Circulation 88:1 -654; Mintz et al., 1994, Circulation 90:1-24). Immunostaining with anti-human IgG antibody demonstrated that the TGF-βR:Fc primarily localized to the adventitia and neointima indicating that these are the predominant sites of TGF-β activity because TGF-βR:Fc binds only active TGF-β. One prominent effect of soluble TGF-βRII was the effect on collagen synthesis, which was particularly striking in the adventitia of Masson's trichrome stained sections. It was further found that the effects of TGF-βR:Fc on collagen expression by Northern blot analysis of RNA isolated from carotid arteries 4 days after injury were markedly reduced for collagen Type I and Type III, but Type XV was unaffected. No differences in levels of osteopontin, tropoelastin, or fibronectin mRNA were detected.

Taken together, the aforementioned findings identify the TGF-β isoforms as major factors mediating adventitial fibrosis and constrictive remodeling following vascular injury. Thus, genes whose expression is affected by TGF-β are likely involved in such TGF-β associated processes, including arterial stenosis mediated by, inter alia, adventitial fibrosis and constrictive remodeling.

Many of the factors and processes involved in artery repair are shared in the development of bone and cartilage. Bone remodeling is a dynamic physiologic process by which bone mass is maintained or adjusted in response to appropriate stimuli (reviewed in Ducy et al., 2000, Science 289:1501-1504). This process consists of two phases, bone resorption by osteoclasts followed by bone deposition by osteoblasts. These events occur continually throughout the skeleton and understanding the factors that regulate this process is likely to have important implications for the treatment of common bone diseases such as osteoporosis. Many vascular injury related genes have been found that are known for their function in bone. These include osteopontin (Giachelli et al., 1993, J. Clin. Invest. 92:1686-1696), alkaline phosphatase (ALP), bone morphogenic proteins (BMPs), and osteocalcin (Balica et al., 1997, Circulation 95:1954-60; Bostrom et al., 1993, J. Clin. Invest. 91:1800-9; Bostrom et al., 1995, Am. J. Cardiol. 75:88B-91B). Calcifications frequently also occur dystrophically in arteries affected by atherosclerosis and it is a characteristic finding in the medial sclerosis of Monckeberg. Furthermore, matrix Gla protein deficient mice typically develop extensive vascular calcifications (Luo et al., 1997, Nature 386:78-81). For a better understanding of potential common denominators between the events taking place in injured arteries or tissues and bone/cartilage development, a brief description of the cellular events is provided here.

Wound healing is characterized by the formation of granulation tissue from connective tissue surrounding the damaged area and its components are inflammatory cells, proliferating fibroblasts and myofibroblasts (smooth muscle α-actin positive), and a rich capillary network. Matrix protein synthesis is abundant and as the wound closes and evolves into a scar, there is a decrease in cellularity associated with the disappearance of myofibroblasts (Desmouliere et al., 1995Am. J. Pathol. 146:56-66). This cell loss occurs via apopototic cell death (Desmouliere et al., 1995, Am. J. Pathol. 146:56-66). A similar course of events can be observed in the adventitia of balloon-injured arteries where the response to injury leads to constrictive remodeling as the predominant cause of restenosis. TGF-β is a major factor mediating the remodeling process in arteries in response to injury (Smith et al., 1999, Circ. Res. 84:1212-1222). Inhibition of TGF-β function with a soluble TGF-β receptor II completely blocks the transdifferentiation of adventitial fibroblasts into myofibroblasts, demonstrating that this transdifferentiation process is at least in part mediated by TGF-β (Smith et al., 1999, Circ. Res. 84:1212-1222).

Chondrocytes participating in endochondral ossification undergo events that have some resemblance to wound healing. These include proliferation in the proliferative zone, followed by hypertrophy and subsequent cell death by apoptosis. The ingrowth of blood vessels in this process is critical as inhibition of vascular endothelial growth factor—(VEGF) mediated angiogenesis causes a decrease in the hypertrophic zone with reduced bone formation (Gerber et al., 1999, Nat. Med. 5:623-628). During this differentiation process matrix protein synthesis is abundant and TGF-β as well as BMP members have been shown to participate in it (Olsen et al., 2000, Annu. Rev. Cell Dev. Biol. 16:191-220).

An important gene for chondrocyte differentiation was first recognized through the discovery of a mutation that is associated with a severe dwarfing syndrome now referred to as campomelic dysplasia (Foster et al., 1994, Nature 372:525-30). This syndrome is characterized by malformation of long bones, vertebrae, pelvic and skull bones. The gene affected was Sox9, a member of the Sox gene family of transcription factors. During mouse development Sox9 transcript expression peaks in cartilage primordia at 11.5 to 14.5 dpc (Ng et al., 1997, Dev. Biol. 183:108-21) and like collagen II expression continues to be high in prechondrocytes and chondrocytes. In the growth plate of long bones, expression of the cartilage specific collagen II and Sox9 are seen in resting and proliferating chondrocytes. In hypertrophic chondrocytes, collagen II is still expressed while Sox9 is turned off. In the absence of Sox9 no chondrocyte specific marker genes are expressed in Sox9 null cells of mouse chimeras (Bi et al., 1999, Nat. Genet. 22:85-89) and no differentiation of chondrocytes from the perichondrium takes place. While other Sox gene family members also participate in chondrocyte specific gene expression Sox9 appears to play a dominant role in this process.

As part of the endochondral ossification process cartilage mass increases by proliferation of chondrocytes as well as deposition of cartilage matrix. Once cartilage is formed chondrocytes located in the central region undergo further maturation into hypertrophic chondrocytes. These chondrocytes are characterized by their withdrawal from the cell cycle and the expression of collagen X which serves as a marker gene for these hypertrophic chondrocytes. With the recruitment of blood vessels primary ossification centers are then formed. The matrix produced by the hypertrophic chondrocytes is subsequently degraded and replaced by trabecular bone synthesized by osteoblasts which are replacing hypertrophic chondrocytes undergoing apoptosis. Simultaneously, a collar of compact bone is formed by osteoblasts located in the perichondrium giving rise to the bone collar around the primary ossification centers. At either end of the cartilage secondary ossification centers originate from the growth plate where a coordinated process of chondrocyte proliferation, maturation, and apoptosis gives rise to longitudinal bone growth.

A member of the hedgehog family of genes, Indian hedgehog (Ihh), has been implicated as a key regulator of bone formation by promoting chondrocyte proliferation and inhibiting chondrocyte hypertrophy (Bitgood et al., 1995, Dev. Biol. 172:126-138). The Ihh deficient mouse has a severe dwarfing syndrome due to the lack of endochondral bone formation (St-Jacques et al., 1999, Genes Dev. 13:2072-2086). Proliferation of chondrocytes is inhibited and chondrocyte maturation is disturbed in these mice. Of interest is the fact that a constitutively active receptor for the parathyroid hormone-related protein (PTHrP) under the control of the collagen IIalpha1 promoter can rescue the chondrocyte maturation phenotype of the Ihh deficient mouse. PTHrP synthesis is stimulated by Ihh expressed by cells in the perichondrium (Vortkamp et al., 1996, Science 273:613-622) and there appears to be a feedback mechanism between these two ligands regulating the relative proportions of hypertrophic and proliferative chondrocytes. Conditions associated with both increased and decreased numbers of proliferative chondrocytes are associated with dwarfism as these result in decreased formation of hypertrophic chondrocytes in both cases.

How expression of Ihh in the proliferating chondrocytes is regulated is currently not well understood. However, signaling via the FGFR3 (Chen et al., 1999, J. Clin. Invest. 104:1517-1525; Naski et al., 1998, Development 125:4977-4988) and the PTHrP receptor may inhibit while BMPs may serve as inducers of Ihh expression (Pathi et al., 1999, Dev. Biol. 209:239-253). It is of interest that Ihh deficiency has very little consequences for osteoblast differentiation occurring during intramembraneous ossification (St-Jacques et al., 1999, Genes Dev. 13:2072-2086).

The transcription factor Cbfal has been identified as a crucial factor regulating osteoblast differentiation in both intramembraneous and endochondral ossification. In the absence of Cbfal, mice develop no bone tissue although a normal cartilage skeleton is formed (Schiffrin, 1995, Cardiology 86:16-22; Komori, et al., 1997, Cell 89:755-764; Otto et al., 1997, Cell 89:765-771). This demonstrates that Cbfal is not essential for chondrogenesis although in certain instances chondrocyte hypertrophy appears to require Cbfal. During embryonic development, Cbfal expression is restricted to cells destined to differentiate either into chondrocytes or osteoblasts (Schiffrin, 1995, Cardiology 86:16-22). Cbfal expression later becomes restricted to osteoblasts with only low levels seen also in hypertrophic chondrocytes (Kim et al., 1999, Mech. Dev. 80:159-170).

It is likely that other transcription factors act downstream of Cbfal since osteoblasts do not appear until 14.5 days post coitus (dpc) while Cbfal expression is detectable as early as 10.5 dpc in mouse embryos. Homeobox genes like Msx2, Hoxa-2 and Bapx1 are likely upstream of Cbfal, regulating its expression (Tribioli et al., 1997, Mech. Dev. 65:145-162; Tribioli et al., 1999, Development 126:5699-5711; Satokata et al., 2000, Nat. Genet. 24:391-395; Gendron-Maguire et al., 1993, Cell 75:1317-1331; Kanzler et al., 1998, Development 125:2587-2597). Deficiency in the homeobox gene Dlx5 in mice is associated with delayed intramembraneous ossification and to a lesser extent also with endochondral ossification (Simeone et al., 1994, Proc. Natl. Acad. Sci. USA 91:2250-2254). However, Cbfal expression is not affected by this deficiency, indicating that Dlx5 is either downstream of Cbfal or in a different signaling pathway. Downstream of Cbfal are several bone matrix proteins. Cbfal is essential for bone matrix production as several bone matrix proteins including collagen I, osteocalcin (an osteoblast specific protein), bone sialoprotein, and alkaline phosphatase have binding sites for Cbfal in their promoters (OSE2 binding site) (Schiffrin, 1995, Cardiology 86:16-22). Several members of the bone morphogenic family (BMP) can induce Cbfal expression in vitro while TGF-β inhibits the expression of Cbfal in osteoblasts in vitro and controls the levels of osteoblast differentiation in vivo (Schiffrin, 1995, Cardiology 86:16-22).

The major structural components of cartilage and bone are collagens I and II with essential roles in providing mechanical strength. Several disease entities are associated with mutations in these types of collagen with phenotypes ranging from normal stature with osteoarthritis to lethality (Alliston et al., 2001, EMBO J. 20:2254-2272; Mundlos et al., 1997, FASEB J. 11:227-233). Collagens play important roles in wound and fracture healing and consequently inhibited collagen formation will impair the healing process. Excessive collagen formation is also of clinical significance in the form of fibrosis in a variety of organs and tissues. Among the many diseases associated with collagen mutations are osteogenesis imperfecta (OI; collagen I) with brittle bones, chondrodysplasias (collagen II), several subtypes of Ehlers-Danlos syndrome (EDS, collagen III and others), Alport syndrome (collagen IV) with nephritis, Bethlem myopathy (collagen VI), dystrophic epidermolysis bullosea (DEB; collagen VII) with separation of the epidermis from the dermis, and many others (for review (Spranger et al., 1994, Eur. J. Pediatr. 153:56-65).

The common structural element in all collagens is a unique triple-helical conformation which can be identified by its repetitive (G-X—Y) amino acid sequence pattern. The conformation is stabilized by glycine as every third residue. The most common mutations are single base substitutions that cause replacement of a single glycine (G) by another amino acid. This results in interruption of the (G-X—Y) sequence pattern and defective folding occurs as a consequence (for review see Myllyharju et al., 2001, Ann. Med. 33:7-21). Collagen synthesis involves a number of post-translational modifications and fibril forming collagens are first synthesized as procollagen molecules with propeptide extensions at both the N— and C-terminal ends.

The intracellular steps involved in the assembly of procollagen molecules from pro α-chains include cleavage of the signal peptide, hydroxylation of certain proline and lysine residues, glycosylation of certain asparagine residues, association of the C propeptides, and formation of interchain and intrachain disulfide bonds. Following association of the C propeptides a nucleus of the triple helix is formed in the C-terminal region and the triple helix is then propagated towards the N terminus in a zipper-like fashion. These C propeptides allow the tethering of the 3 chains thus promoting triple helix formation, a process that can also be performed by a transmembrane domain (Baum et al., 1999, Curr. Opin. Struct. Biol. 9:122-128). The hydroxylation of proline residues in the Y position creates a unique region of 5-6 consecutive G-X-hydroxyP (proline) triplets at the C terminus of the triple-helix domain which are thought to act as a nucleation site for bringing the 3 chains into the correct dihedral angles and for forming the correct interchain hydrogen bonds. Studies by Bulleid et al. (Bulleid et al., 1997, EMBO J. 16:6694-6701) demonstrated that this hydroxyP rich region is required for nucleation but that 2 consecutive G-X-hydroxyP triplets at the C terminus may be sufficient. During their transport from the endoplasmic reticulum (ER) through the Golgi stacks lateral aggregation of the procollagen molecules and increased condensation of the aggregates occurs during this process resulting in granule formation for secretion. In the extracellular space the N and C propeptides are cleaved and self-assembly of the collagen molecules into fibrils occurs followed by covalent cross-linking of collagen molecules (reviewed in Myllyharju et al., 2001, Ann. Med. 33:7-21).

Less abundant collagens in cartilage are collagens IX, X, and XI where collagens IX and XI have predominantly regulatory functions in fibril assembly. Expression of collagen X is limited to hypertrophic chondrocytes in the endochondral ossification process (Alliston et al., 2001, EMBO J. 20:2254-2272). Important cartilage proteoglycans include perlecan and aggrecan. The latter forms large complexes with hyaluronic acid stabilized via link protein (Bulleid et al., 1997, EMBO J. 16:6694-6701). Mutations in both link protein as well as aggrecan are associated with multiple skeletal abnormalities (Watanabe et al., 1998, J. Biochem. (Tokyo) 124:687-693; Watanabe et al., 1994, Nat. Genet. 7:154-157).

Arterial stenosis with reduction in blood flow is a common problem in many vascular diseases and it is an important causal factor in the morbidity and mortality associated with these diseases. Despite the fact that various growth factors, especially TGF-β, have been implicated in arterial stenosis, very few factors involved in arterial stenosis have been identified and characterized. Nevertheless, the identification of such factors is crucial in the development of diagnostics and therapeutics for treatment of vascular diseases associated or mediated by arterial stenosis. Thus, there is long-felt need for the identification and characterization of factors associated with arterial stenosis. The present invention meets this need.

Similarly, proper formation of bone and cartilage is essential to normal development, and the formation of collagen matrices is an integral part of this process. Indeed, mutations of collagen genes are among the most common resulting in skeletal abnormalities. Effective treatment of bone/cartilage and vascular diseases is dependent on understanding the processes by which these systems develop. Therefore, despite the fact that little is known about the factors that modulate collagen matrices, understanding this process is highly relevant to many human diseases involving virtually all organ systems. Thus, there is a long felt need for the identification and characterization of factors associated with bone formation. The present invention meets this need.

Bone morphogenetic protein 1 (BMP1) is involved in the processing of fibrillar collagens type I, II, and III by cleaving the C terminal propeptide (Kessler et al., 1996, Science 271:360-362). BMP1 has also been found to cleave the propeptide of lysyl-oxidase, an enzyme involved in cross-linking of collagen fibrils (Uzel et al., 2001, J. Biol. Chem. 276:22537-22543). Cleavage of these propeptides is important for proper collagen formation. BMP1 also degrades chordin, which functions as an antagonist of bone morphogenetic proteins (BMPs) (Wardle et al., 1999, Mech. Dev. 86:75-85). Therefore, understanding the role(s) of the bone morphogenetic proteins, especially BMP1, in bone and collagen related processes is important in developing methods for affecting these processes. Affecting processes mediated by BMP1 can provide important therapeutics for treating or alleviating a condition, disease or disorder involving such processes. Thus, there is a long-felt need to understand and regulate mechanisms mediated by BMP1, including bone matrix formation and collagen deposition, and the present invention meets these needs.

Likewise, bone morphogenetic protein 4 (BMP4) plays critical roles in many organ systems and in cell differentiation processes. For instance, the development of the central nervous system and skeletal systems depends upon BMP4, as does the differentiation of stem cells. BMPs are also used to promote bone and cartilage formation in orthopedic settings of fracture healing and bone/cartilage repair. Thus, understanding and affecting processes mediated by BMP4 can provide important therapeutics for treating or alleviating a condition, disease or disorder involving such processes, including, but not limited to, development of organ systems and differentiation of cells, especially stem cells. Because regulating BMP4 expression can provide an important therapeutic benefit for promoting bone and cartilage growth in any orthopedic application where skeletal repair/healing is sought and in promoting the differentiation of stem cells into different cell lineages, there is a long-felt need to understand and regulate mechanisms mediated by BMP4 and for affecting BMP4 expression, and the present invention meets these needs.

Further, there is a long-felt need for methods to affect expression of osteopontin (OPN), which is another important protein relating to bone and cartilage formation, and the present invention meets this need.

### BRIEF SUMMARY OF THE INVENTION

The invention includes an isolated nucleic acid encoding a human cleaved CTHRC1.

In one aspect, the nucleic acid further comprises a nucleic acid encoding a tag polypeptide covalently linked thereto.

In yet another aspect, the tag polypeptide is selected from the group consisting of a green fluorescent protein tag polypeptide, an influenza virus hemagglutinin tag polypeptide, a myc tag polypeptide, a glutathione-S-transferase tag polypeptide, a myc-pyruvate kinase tag polypeptide, a His6 tag polypeptide, a FLAG tag polypeptide, and a maltose binding protein tag polypeptide.

In a further aspect, the nucleic acid further comprises a nucleic acid specifying a promoter/regulatory sequence operably linked thereto.

In another aspect, the nucleic acid shares at least about 33% sequence identity with a nucleic acid encoding at least one of cleaved CTHRC1 longer fragment (SEQ ID NO:10), and a human cleaved CTHRC1 shorter fragment (SEQ ID NO:12).

The invention includes an isolated nucleic acid encoding a human cleaved CTHRC1, wherein the amino acid sequence of the human cleaved CTHRC1 shares at least about 33% sequence identity with an amino acid sequence of at least one of SEQ ID NO:11, and SEQ ID NO:13.

The invention also includes an isolated polypeptide comprising a human cleaved CTHRC1.

In one aspect, the human cleaved CTHRC1 shares at least about 6% sequence identity with an amino acid sequence of at least one of SEQ ID NO:11, and SEQ ID NO:13.

In another aspect, the invention includes a vector comprising an isolated nucleic acid encoding a human cleaved CTHRC1.

In a further aspect, the vector further comprises a nucleic acid specifying a promoter/regulatory sequence operably linked thereto.

The invention includes a recombinant cell comprising an isolated nucleic acid encoding a human cleaved CTHRC1.

The invention includes a recombinant cell comprising a vector comprising an isolated nucleic acid encoding a human cleaved CTHRC1.

The invention includes an isolated nucleic acid complementary to an isolated nucleic acid encoding a human cleaved CTHRC1, the complementary nucleic acid being in an antisense orientation.

In one aspect, the nucleic acid shares at least about 33% identity with a nucleic acid complementary with a nucleic acid having the sequence of at least one of a human cleaved CTHRC1 longer fragment (SEQ ID NO:10), and a human cleaved CTHRC1 shorter fragment (SEQ ID NO:12).

In another aspect, the invention includes a recombinant cell comprising the isolated nucleic.

In yet another aspect, the invention includes an antibody that specifically binds with an isolated polypeptide comprising a human cleaved CTHRC1.

In one aspect, the antibody is selected from the group consisting of a polyclonal antibody, a monoclonal antibody, a humanized antibody, a chimeric antibody, and a synthetic antibody.

The invention includes a composition comprising an isolated nucleic acid complementary to an isolated nucleic acid encoding a human cleaved CTHRC1, the complementary nucleic acid being in an antisense orientation, and a pharmaceutically-acceptable carrier.

The invention includes a composition comprising an isolated nucleic acid encoding a human cleaved CTHRC1, and a pharmaceutically-acceptable carrier.

The invention includes a composition comprising an isolated polypeptide comprising a human cleaved CTHRC1, and a pharmaceutically-acceptable carrier.

The invention includes a transgenic non-human mammal comprising an isolated nucleic acid encoding a human cleaved CTHRC1.

The invention also includes a method of treating a disease mediated by collagen matrix production in a human in need thereof. The method comprises administering to a human afflicted with the disease an effective amount of CTHRC1, thereby treating the disease mediated by collagen matrix production in the human.

In one aspect, CTHRC1 is administered as a molecule selected from the group consisting of a CHTRC1 polypeptide and a nucleic acid encoding CTHRC1.

In another aspect, the disease is selected from the group consisting of fibrosis, constrictive remodeling, and restenosis.

In a further aspect, the fibrosis is fibrosis of an organ.

In yet another aspect, the organ is at least one organ selected from the group consisting of kidney, lung, liver and skin.

The invention includes a method of treating constrictive remodeling in a human in need therefor. The method comprises administering to a human an effective amount of CTHRC1, thereby treating the constrictive remodeling in the human.

The invention further includes a method of preventing constrictive remodeling in a human in need therefor. The method comprises administering to a human an effective amount of CTHRC1, thereby preventing the constrictive remodeling in the human.

The invention includes a method of treating restenosis in a human in need therefor. The method comprises administering to a human an effective amount of CTHRC1, thereby treating restenosis in the human.

The invention includes a method of preventing restenosis in a human in need therefor. The method comprises administering to a human an effective amount of CTHRC1, thereby preventing restenosis in the human.

The invention includes a method of treating fibrosis in a human in need therefor. The method comprises administering to a human an effective amount of CTHRC1, thereby treating fibrosis in the human.

The invention includes a method of preventing fibrosis in a human in need therefor. The method comprises administering to a human an effective amount of CTHRC1, thereby preventing fibrosis in the human.

The invention includes a kit for treating a disease mediated by collagen matrix formation in a human in need therefor. The kit comprises an effective amount of CTHRC1, an applicator, and an instructional material for the use thereof.

In one aspect, the disease is selected from the group consisting of constrictive remodeling, arterial restenosis, vessel injury, and fibrosis.

The invention includes a kit for preventing a disease mediated by collagen matrix formation in a human in need therefor. The kit comprises an effective amount of CTHRC1, an applicator, and an instructional material for the use thereof.

The invention includes an isolated nucleic acid encoding a mutant CTHRC1, wherein the nucleic acid comprises a nucleotide sequence encoding a human CTHRC1 collagen domain is replaced by a nucleotide sequence of mouse collagen 1 alpha 1 encoding a mouse collagen 1 alpha 1 collagen domain.

In one aspect, the nucleotide sequence encoding the human CTHRC1 collagen domain is SEQ ID NO:15 and further wherein the nucleotide sequence encoding the mouse collagen 1 alpha 1 collagen domain is SEQ ID NO:17.

The invention includes an isolated mutant CTHRC1 polypeptide, wherein the polypeptide comprises substitution of a human CTHRC1 collagen domain with a mouse collagen 1 alpha 1 collagen domain.

In one aspect, the amino acid sequence of the human CTHRC1 collagen domain is SEQ ID NO:14 and further wherein the amino acid sequence of the mouse collagen 1 alpha 1 collagen domain is SEQ ID NO:16.

The invention includes a method of decreasing the level of BMP1 in a cell. The method comprises contacting a cell expressing BMP1 with a BMP1 inhibiting amount of collagen triple helix repeat containing 1 (CTHRC1), thereby decreasing the level of BMP1 in the cell.

The invention includes a method of decreasing the level of BMP1 mRNA in a cell. The method comprises contacting a cell with a BMP1 mRNA expression-inhibiting amount of CTHRC1, thereby decreasing the level of BMP1 mRNA in the cell.

The invention includes a method of increasing the level of BMP1 in a cell. The method comprises contacting a cell expressing BMP1 with a BMP1 increasing amount of a collagen triple helix repeat containing 1 (CTHRC1) inhibitor, thereby increasing the level of BMP1 in the cell.

The invention also includes a method of increasing the level of BMP1 mRNA in a cell. The method comprises contacting a cell with a BMP1 mRNA expression-increasing amount of a CTHRC1 inhibitor, thereby increasing the level of BMP1 mRNA in the cell.

The invention includes a method of increasing the level of a propeptide in a cell. The method comprises contacting a cell with a BMP1 inhibiting amount of CTHRC1, thereby increasing the level of the propeptide in the cell.

In one aspect, the propeptide is selected from the group consisting of a procollagen and a propeptide of lysyl-oxidase.

The invention includes a method of inhibiting collagen formation by a cell. The method comprises contacting the cell with a BMP1 inhibiting amount of CTHRC1, thereby inhibiting collagen formation by the cell.

The invention includes a method of decreasing bone matrix formation by a cell. The method comprises contacting a cell with a BMP1 inhibiting amount of CTHRC1, thereby decreasing bone matrix formation by the cell.

The invention includes a method of decreasing the level of collagen in a cell. The method comprises contacting a cell with a BMP1 inhibiting amount of CTHRC1, thereby decreasing the level of collagen in the cell.

In one aspect, the collagen is type I collagen.

The invention includes a method of increasing the level of procollagen in a cell. The method comprises contacting a cell with a BMP1 inhibiting amount of CTHRC1, thereby increasing the level of procollagen in the cell.

The invention includes a method of increasing the level of chordin in a cell. The method comprises contacting a cell with a BMP1 inhibiting amount of CTHRC1, thereby increasing the level of chordin in the cell.

The invention includes a method of inhibiting cross-linking of collagen fibrils in a cell. The method comprises contacting a cell with a BMP1 inhibiting amount of CTHRC1, wherein BMP1 is responsible for processing a propeptide lysyl-oxidase, and further wherein the lysyl-oxidase mediates cross-linking the collagen fibrils, thereby inhibiting cross-linking of collagen fibrils in the cell.

The invention includes a method of inhibiting plaque rupture in a blood vessel. The method comprises administering a collagen matrix production enhancing amount of a CTHRC1 inhibitor to a blood vessel comprising a plaque, thereby inhibiting plaque rupture in the blood vessel.

In one aspect, the CTHRC1 inhibitor is selected from the group consisting of an antibody that specifically binds with CTHRC1 and a CTHRC1 antisense nucleic acid.

The invention includes a method of identifying a compound that affects collagen production in a cell. The method comprises contacting a cell comprising CTHRC1 with a test compound and assessing the level of CTHRC1 in the cell, wherein a higher or lower level CTHRC1 in the cell contacted with the test compound compared with the level of CTHRC1 in a second otherwise identical cell not contacted with the test compound is an indication that the test compound inhibits collagen production in the cell, thereby identifying a compound that inhibits collagen production in the cell.

In one aspect, the invention includes a compound identified by this method.

In another aspect, the test compound affects the level of BMP1 in the cell.

In a further aspect, the test compound affects the level of BMP1 mRNA in the cell.

In yet another aspect, the test compound affects the level of procollagen in the cell.

In yet a further aspect, the test compound affects the level of chordin in the cell.

The invention includes a method of decreasing collagen formation in a mammal in need thereof. The method comprises administering a BMP1 inhibiting amount of CTHRC1 to the mammal, whereby inhibiting BMP1 reduces collagen production, thereby decreasing collagen formation in the mammal.

In one aspect, the mammal has a condition mediated by collagen formation and further wherein the condition is selected from the group consisting of wound scarring, wound healing, keloid formation, inflammation-associated scarring, pulmonary fibrosis, and angioplasty-associated vascular fibrosis.

The invention includes a method of increasing bone matrix production in a cell. The method comprises administering an effective amount of an inhibitor of CTHRC1 to the cell, wherein inhibition of CTHRC1 increases the level of BMP1 in the cell, and further wherein increasing the level of BMP1 increases bone matrix production, thereby increasing bone matrix production in the cell.

The invention also includes a method of increasing collagen production in a mammal in need thereof. The method comprises administering an effective amount of an inhibitor of CTHRC1 to the mammal, wherein inhibition of CTHRC1 increases the level of BMP1 in the mammal, and further wherein increasing the level of BMP1 increases processing of fibrillar collagen, thereby increasing collagen production in the mammal.

The invention includes a method of treating a disease mediated by expression of BMP1 in a mammal in need thereof. The method comprises administering to the mammal a BMP1 inhibiting amount of CTHRC1, thereby treating the disease mediated by expression of BMP1 in the mammal.

The invention includes a kit for decreasing the level of BMP1 in a cell. The kit comprises a BMP1 inhibiting amount of collagen triple helix repeat containing 1 (CTHRC1). The kit further comprises an applicator, and an instructional material for the use thereof.

The invention includes a kit for decreasing the level of BMP1 mRNA in a cell. The kit comprises a BMP1 inhibiting amount of collagen triple helix repeat containing 1 (CTHRC1). The kit further comprises an applicator, and an instructional material for the use thereof.

The invention includes a kit for increasing the level of BMP1 in a cell. The kit comprises a BMP1 increasing amount of a collagen triple helix repeat containing 1 (CTHRC1) inhibitor. The kit further comprises an applicator, and an instructional material for the use thereof.

The invention includes a kit for increasing the level of a propeptide in a cell. The kit comprises a BMP1 inhibiting amount of CTHRC1. The kit further comprises an applicator, and an instructional material for the use thereof.

In one aspect, the propeptide is selected from the group consisting of a procollagen and a propeptide of lysyl-oxidase.

The invention further includes a kit for inhibiting collagen formation by a cell. The kit comprises a BMP1 inhibiting amount of CTHRC1. The kit further comprises an applicator, and an instructional material for the use thereof.

The invention includes a kit for decreasing bone matrix formation by a cell, the kit comprising a BMP1 inhibiting amount of CTHRC1, the kit further comprising an applicator, and an instructional material for the use thereof.

The invention includes a kit for decreasing the level of collagen in a cell. The kit comprises a BMP1 inhibiting amount of CTHRC1. The kit further comprises an applicator, and an instructional material for the use thereof.

In one aspect, the collagen is type I collagen.

The invention includes a method of increasing the level of bone morphogenetic protein 4 (BMP4) in a cell. The method comprises contacting a cell expressing BMP4 with a collagen triple helix repeat containing 1 (CTHRC1) in an amount sufficient to increase the level of the BMP4 in the cell, thereby increasing the level of BMP4 in the cell.

The invention includes a method of increasing the level of BMP4 promoter activity in a cell. The method comprises contacting a cell with CTHRC1 in an amount sufficient to increase the level of the BMP4 promoter activity in the cell, thereby increasing the level of BMP4 promoter activity in the cell.

The invention includes a method of promoting bone growth in a mammal. The method comprises contacting a mammal with CTHRC1 in an amount sufficient to increase the level of BMP4 in the mammal, thereby promoting bone growth in the mammal.

The invention includes a method of promoting differentiation of a stem cell. The method comprises contacting the stem cell with CTHRC1 in an amount sufficient to increase the level of BMP4 in the stem cell, thereby promoting differentiation of the stem cell.

The invention includes a method of decreasing the level of osteopontin (OPN) in a cell. The method comprises contacting the cell with CTHRC1 in an amount sufficient to increase the level of BMP4, thereby decreasing the level of OPN in the cell.

The invention includes a method of increasing the level of OPN in a cell. The method comprises contacting the cell with a CTHRC1 inhibiting amount of a CTHRC1 inhibitor, thereby increasing the level of OPN in the cell.

The invention includes a method of identifying a compound that effects a CTHRC1 -mediated reduction of BMP4 in a cell. The method comprises contacting a CTHRC1-containing cell with a test compound, wherein a lower level of BMP4 in the cell contacted with the test compound compared with the level of BMP4 in a second otherwise identical cell not contacted with the test compound is an indication that the test compound reduces the level of BMP4 in the cell, and further wherein the test compound affects the activity of CTHRC1, thereby identifying a compound that effects a CTHRC1-mediated reduction of BMP4 in the cell.

The invention includes a method of treating a disease mediated by BMP4 in a human in need thereof. The method comprises administering to a human afflicted with a disease mediated by BMP4 a CTHR1 inhibiting amount of a CTHRC1 inhibitor, thereby treating the disease mediated by BMP4 in the human in need thereof.

The invention also includes a method of treating a disease mediated by under-expression of BMP4 in a human in need thereof. The method comprises administering to a human afflicted with the disease a BMP4 expression-inducing amount of CTHRC1.

The invention includes a method of increasing the level of a muscle segment homeobox 1 (Msx 1) in a cell. The method comprises contacting a cell expressing BMP4 with a collagen triple helix repeat containing 1 (CTHRC1) in an amount sufficient to increase the level of the BMP4 in the cell, wherein increasing the level of BMP4 mediates an increase in the level of Msx1, thereby increasing the level of Msx1 in the cell.

The invention includes a kit for increasing the level of bone morphogenetic protein 4 (BMP4) in a cell. The kit comprises an amount of collagen triple helix repeat containing 1 (CTHRC1) sufficient to increase the level of the BMP4 in the cell. The kit further comprises an applicator, and an instructional material for the use thereof.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that invention is not limited to the precise arrangements and instrumentalities shown. In the drawings:

FIG. 1A-1 is an image of a Northern blot depicting total RNA isolated from various rat organs probed with labeled CTHRC1 (REMODEL) cDNA. A significant 1.2 kb band was present in cultured rat aortic smooth muscle cells, ballooned rat aorta, lung, and brain. Significantly lower CTHRC1 mRNA levels were detected in other tissues (i.e., liver, thymus, spleen, kidney, heart, muscle, uterus, and testis). A transcript of about 3.5 kb was detected in SMC upon longer exposure of the blot or loading of higher amount of RNA on the gel.

FIG. 1A-2 is an image depicting the gel used for Northern blot analysis in FIG. 1A-1. The gel was stained with ethidium bromide.

FIG. 1B-1 is an image of a Northern blot depicting expression of CTHRC1 mRNA in 8 day balloon-injured rat carotid arteries and normal carotid arteries. The data depicted herein demonstrate that CTHRC1 mRNA is expressed in the injured arteries only.

FIG. 1B-2 is an image depicting the gel used for Northern blot analysis in FIG. 1B-1. The gel was stained with ethidium bromide.

FIG. 1C-1 is an image of a Northern blot depicting levels of CTHRC1 mRNA in MC3T3 cells. The data disclosed herein demonstrate that the levels of CTHRC1 (REMODELIN) mRNA were increased by the addition of bone morphogenetic protein-4 (BMP-4) with peak expression after 8 hours.

FIG. 1C-2 is an image of a Northern blot depicting levels of CTHRC1 (“remodel”) mRNA in MC3T3 cells. The data disclosed herein demonstrate that the levels of CTHRC1 mRNA were increased by the addition of TGF-β, with peak expression after 8 hours.

FIG. 1C-3 is an image depicting the gel used for Northern blot analysis in FIG. 1C-2. The gel was stained with ethidium bromide.

FIG. 2A is an image depicting an in situ hybridization analysis using [³⁵S]-UTP labeled antisense CTHRC1 riboprobe (sequence, SEQ ID NO:6). The arrowheads indicate the position of the internal elastic lamina. The image depicts normal carotid arteries and demonstrates no detectable CTHRC1 expression therein. The presence of silver grains, appearing as white specks under dark field illumination, indicates CTHRC1 expression.

FIG. 2B is an image depicting an in situ hybridization analysis using [³⁵S]-UTP labeled antisense CTHRC1 riboprobe (SEQ ID NO:6). The arrowheads indicate the position of the internal elastic lamina. The image demonstrates strong CTHRC1 expression limited to the adventitia of 8 day balloon injured arteries. The presence of silver grains, appearing as white specks under dark field illumination, indicates CTHRC1 expression.

FIG. 2C is an image depicting an in situ hybridization analysis using [³⁵S]-UTP labeled antisense CTHRC1 riboprobe (SEQ ID NO:6). The arrowheads indicate the position of the internal elastic lamina. The image demonstrates maintained but decreased CTHRC1 expression in the adventitia two weeks post-injury. The presence of silver grains, appearing as white specks under dark field illumination, indicates CTHRC1 expression.

FIG. 2D is an image depicting an in situ hybridization analysis using [³⁵S]-UTP labeled antisense CTHRC1 riboprobe (SEQ ID NO:6). The arrowheads indicate the position of the internal elastic lamina. The image demonstrates that at 4 weeks post-injury, expression levels of CTHRC1 expression were similar to levels detected in normal, control vessels. The presence of silver grains, appearing as white specks under dark field illumination, indicates CTHRC1 expression.

FIG. 2E is an image depicting an in situ hybridization analysis using [³⁵S]-UTP labeled antisense CTHRC1 riboprobe (SEQ ID NO:6). The image depicts a transverse section of an 11.5 days post coitus (dpc) mouse embryo wherein CTHRC1 expression is detectable in the developing mesoderm. The presence of silver grains, appearing as white specks under dark field illumination, indicates CTHRC1 expression.

FIG. 2F is an image depicting an in situ hybridization analysis using [³⁵S]-UTP labeled antisense CTHRC1 riboprobe (sequence, SEQ ID NO:6). The image depicts a 14.5 dpc mouse embryo expressing CTHRC1 in developing bone. The developing brain and bone are depicted. The image depicts that CTHRC1 expression becomes limited to the developing bone at later stages of embryo development. The presence of silver grains, appearing as white specks under dark field illumination, indicates CTHRC1 expression.

FIG. 2G is an image depicting an in situ hybridization analysis using [³⁵S]-UTP labeled antisense CTHRC1 riboprobe (SEQ ID NO:6). The image depicts CTHRC1 expression in the bones of the snout in a 14.5 dpc embryo. The presence of silver grains, appearing as white specks under dark field illumination, indicates CTHRC1 expression.

FIG. 2H is an image depicting an in situ hybridization analysis using [³⁵S]-UTP labeled antisense CTHRC1 riboprobe (SEQ ID NO:6). The image depicts strong detectable CTHRC1 expression in the bone of the developing skull of a 14.5 dpc mouse embryo. The presence of silver grains, appearing as white specks under dark field illumination, indicates CTHRC1 expression.

FIG. 21 is an image depicting an in situ hybridization analysis using [³⁵S]-UTP labeled antisense CTHRC1 riboprobe (SEQ ID NO:6). The image depicts osteoblasts adjacent to mineralized bone in a femur from a rat pup depicting expression of CTHRC1 mRNA. The arrowheads indicate the transition from the osteoblast layer to the mineralized bone layer at the upper right portion of the image. The image demonstrates strong CTHRC1 expression in osteoblasts along mineralized bone. The presence of silver grains, appearing as white specks under dark field illumination, indicates CTHRC1 expression.

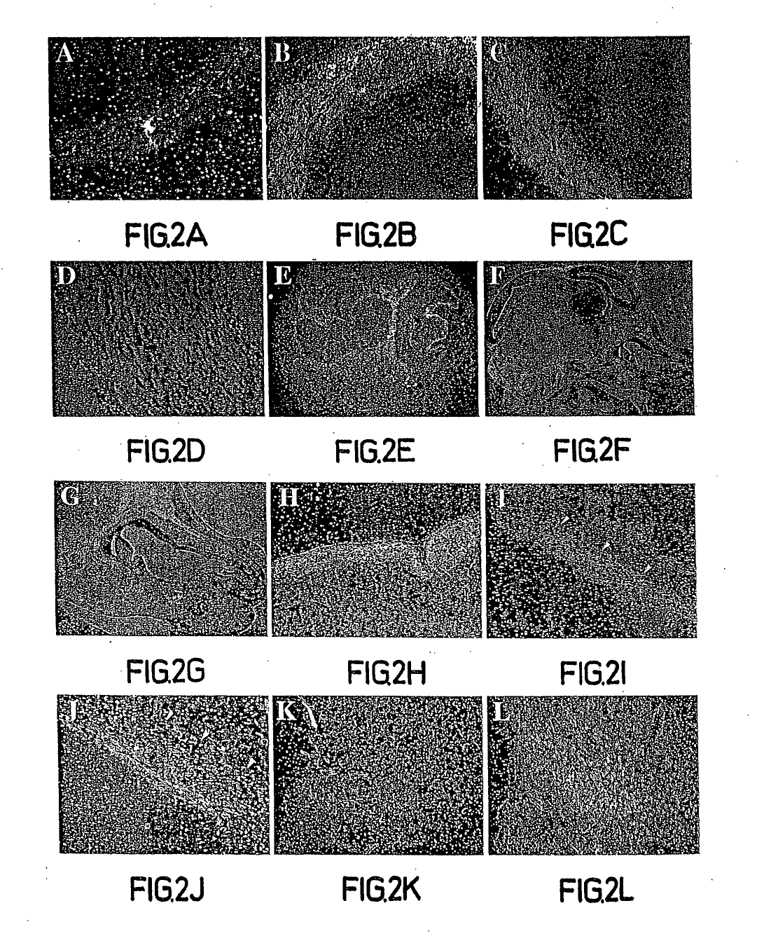

FIG. 2J is an image depicting an in situ hybridization analysis using [³⁵S]-UTP labeled antisense CTHRC1 riboprobe (SEQ ID NO:6). The image depicts osteoblasts adjacent to mineralized bone in a femur from a rat pup depicting expression of CTHRC1 mRNA. The arrowheads indicate the transition from the osteoblast layer to the mineralized bone layer at the upper right portion of the image. The image demonstrates strong CTHRC1 expression in osteoblasts along mineralized bone. The presence of silver grains, appearing as white specks under dark field illumination, indicates CTHRC1 expression.

FIG. 2K is an image depicting an in situ hybridization analysis using [³⁵S]-UTP labeled antisense CTHRC1 riboprobe (SEQ ID NO:6). The image depicts expression of CTHRC1 was undetectable in normal skin (the skin surface is located on the left side of the image). The presence of silver grains, appearing as white specks under dark field illumination, indicates CTHRC1 expression.

FIG. 2L is an image depicting an in situ hybridization analysis using [³⁵S]-UTP labeled antisense CTHRC1 riboprobe (SEQ ID NO:6). The image depicts extensive expression of CTHRC1 in a 7 day old skin incision along the wound edge in (myo)fibroblasts of the granulation tissue. The presence of silver grains, appearing as white specks under dark field illumination, indicates CTHRC1 expression.

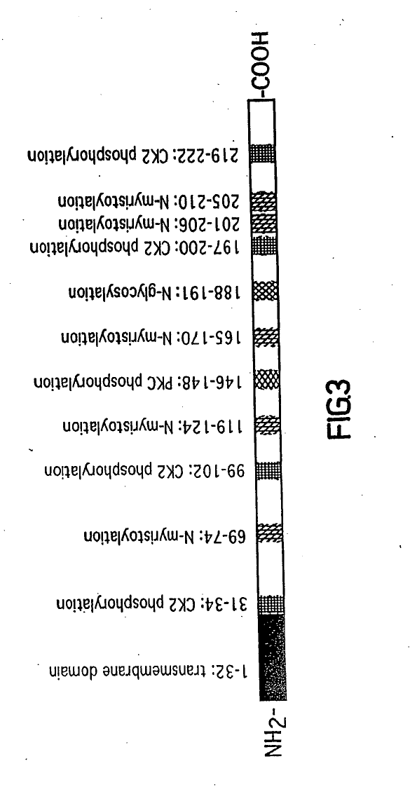

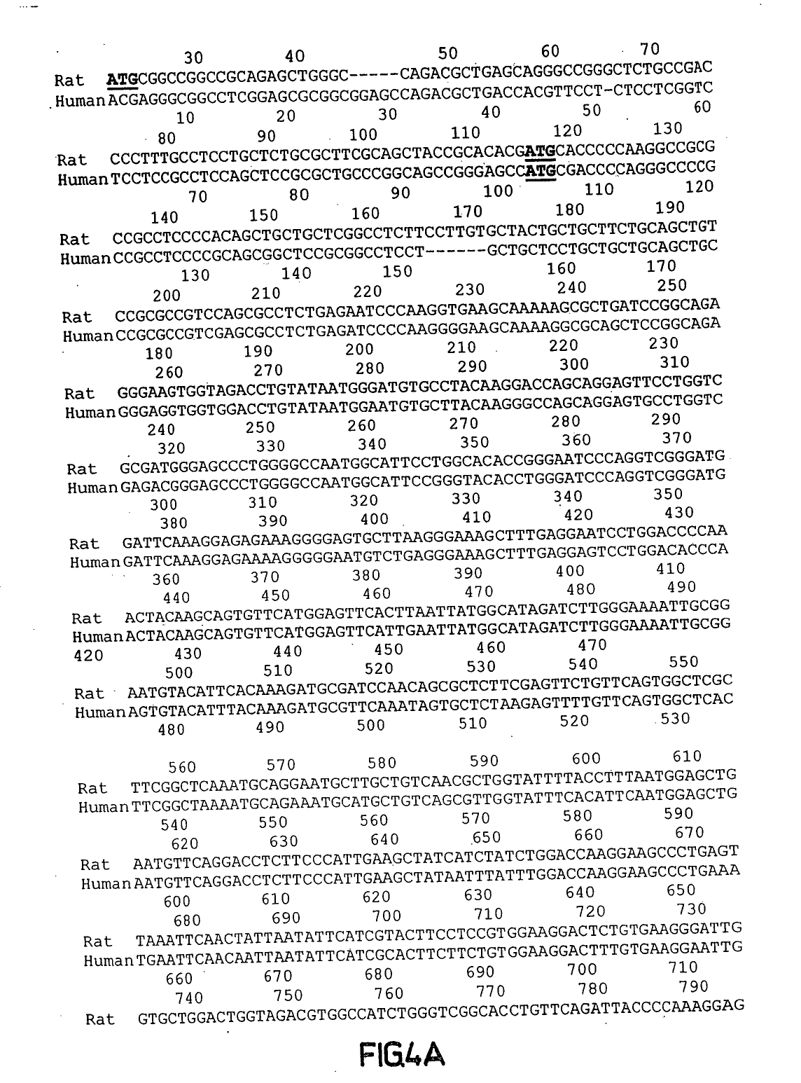

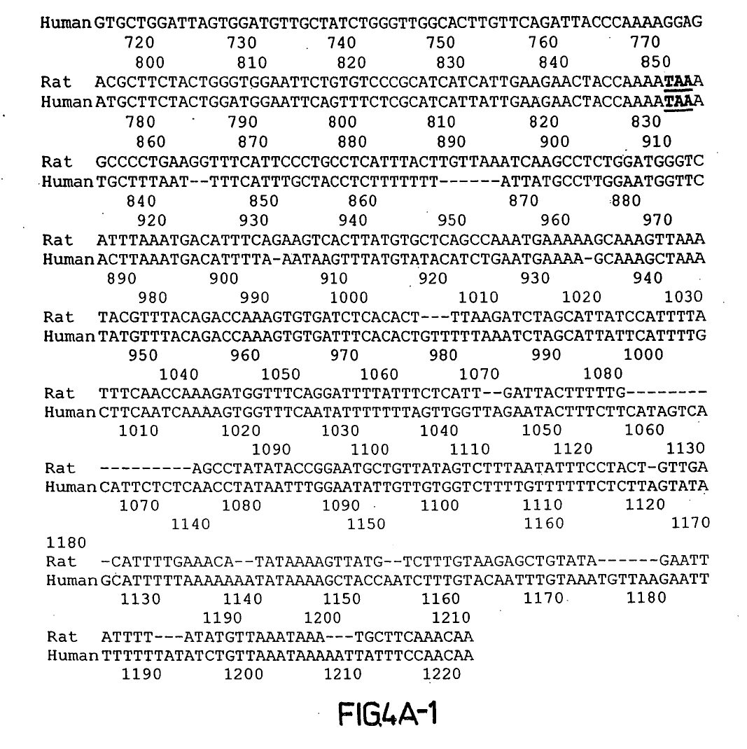

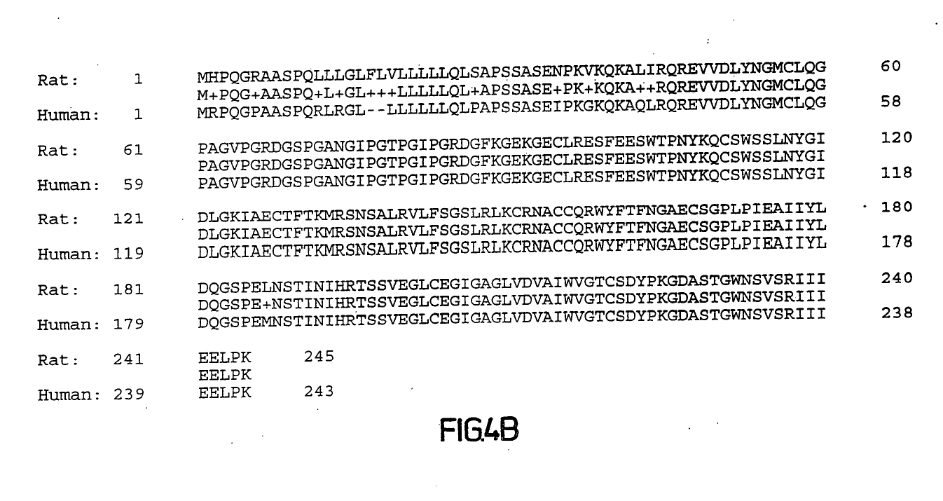

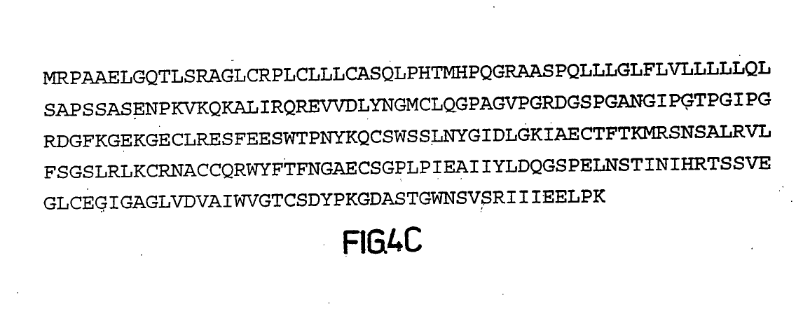

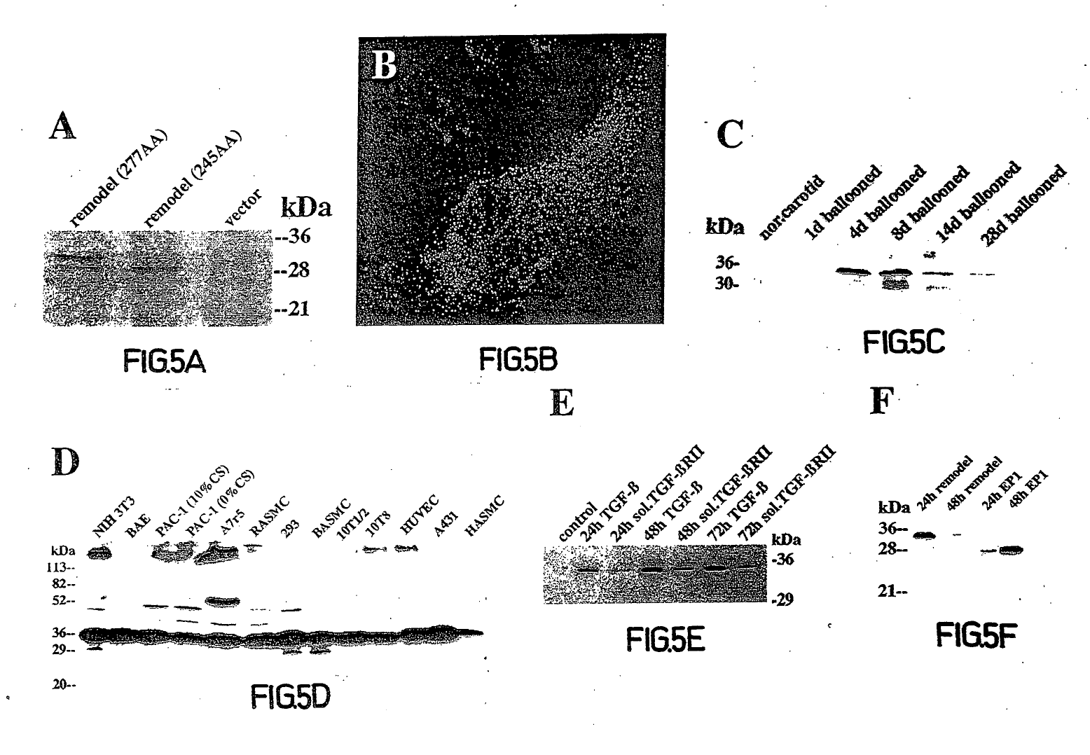

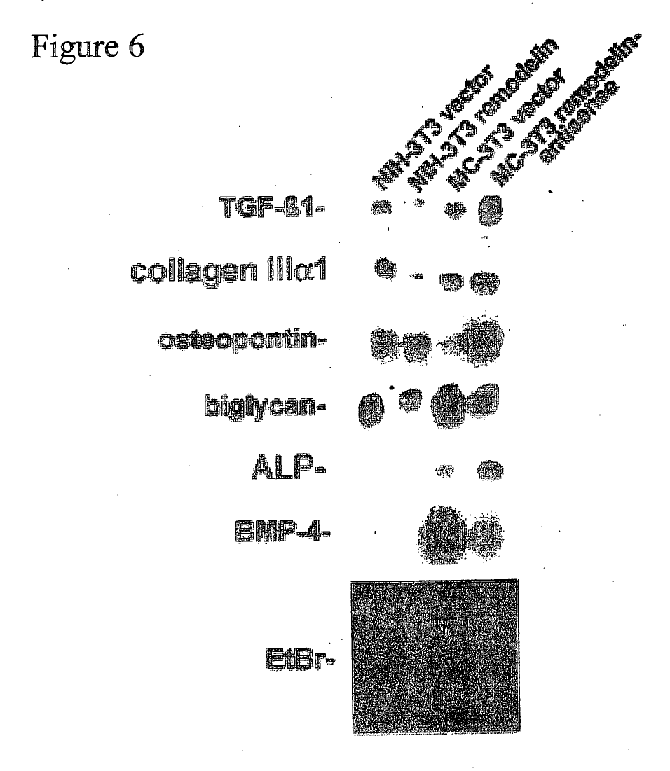

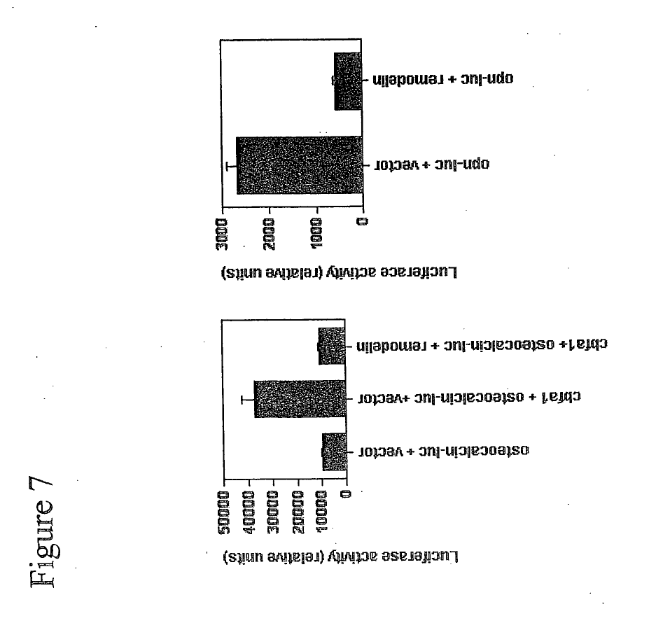

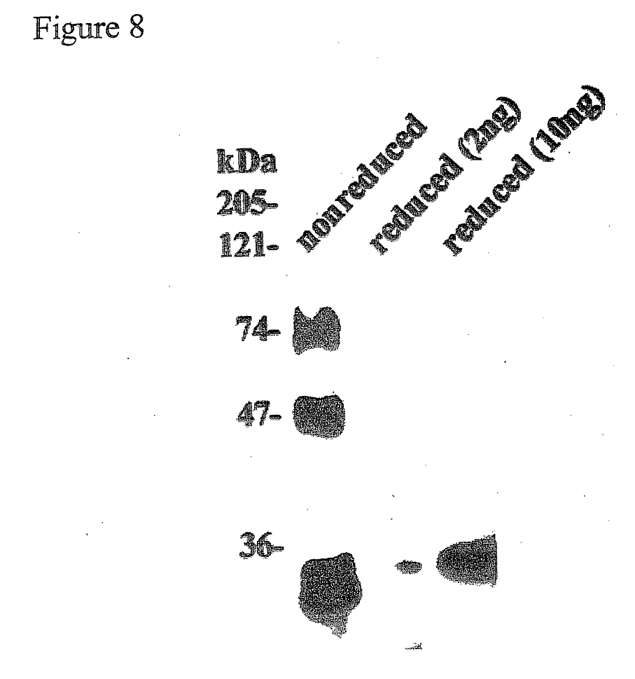

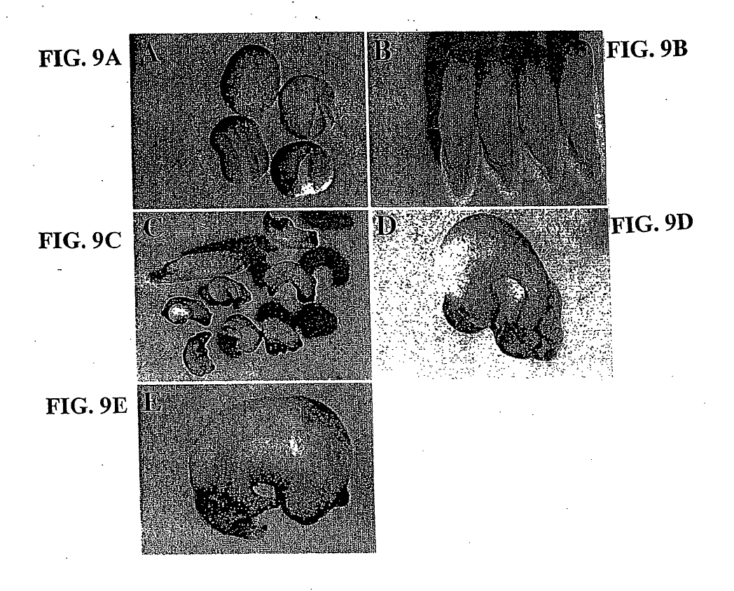

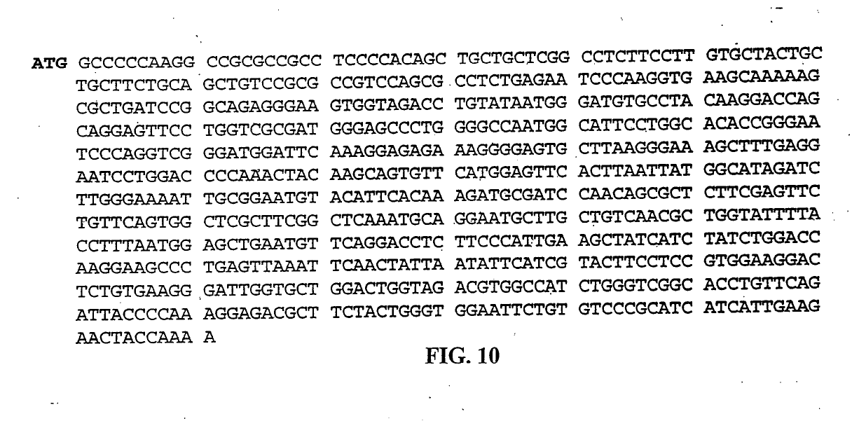

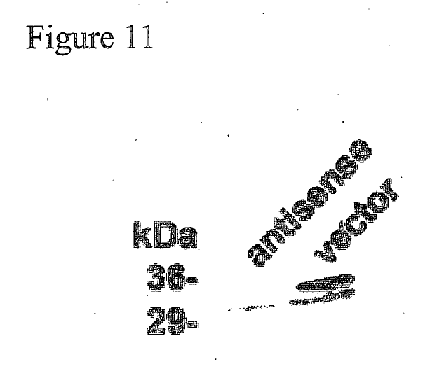

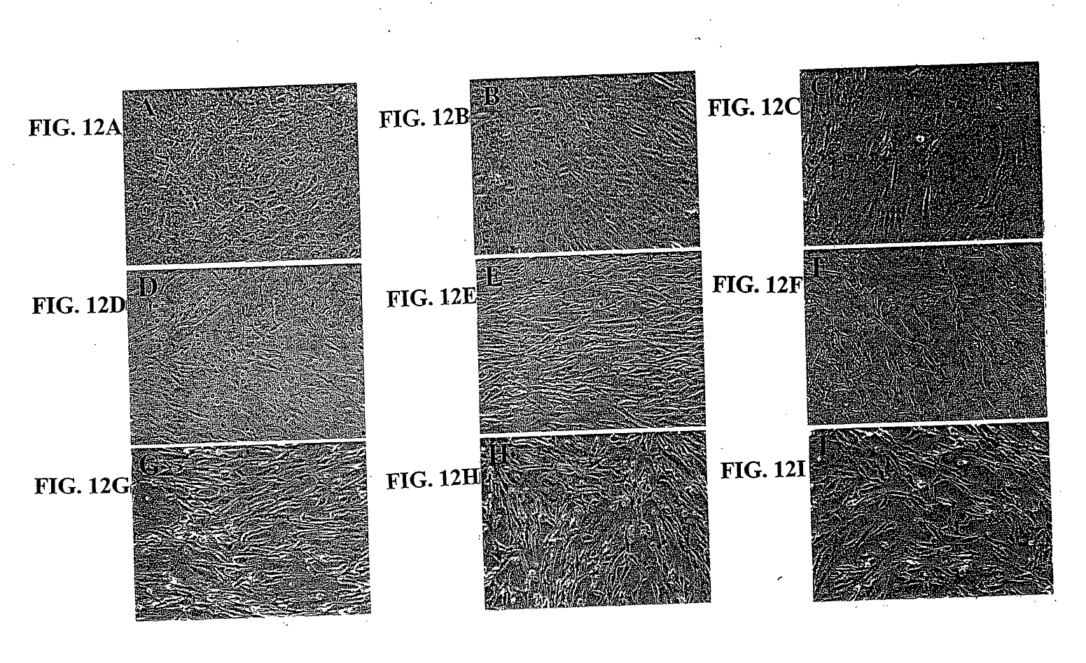

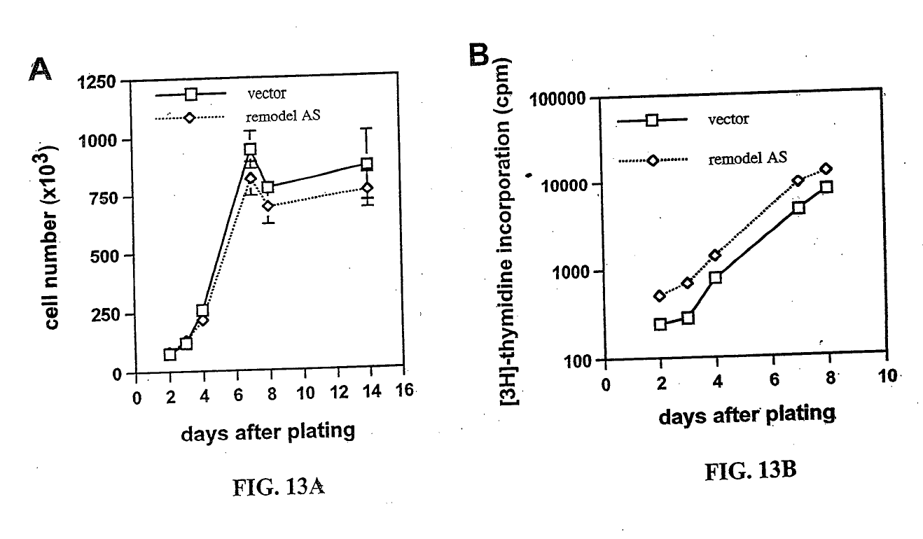

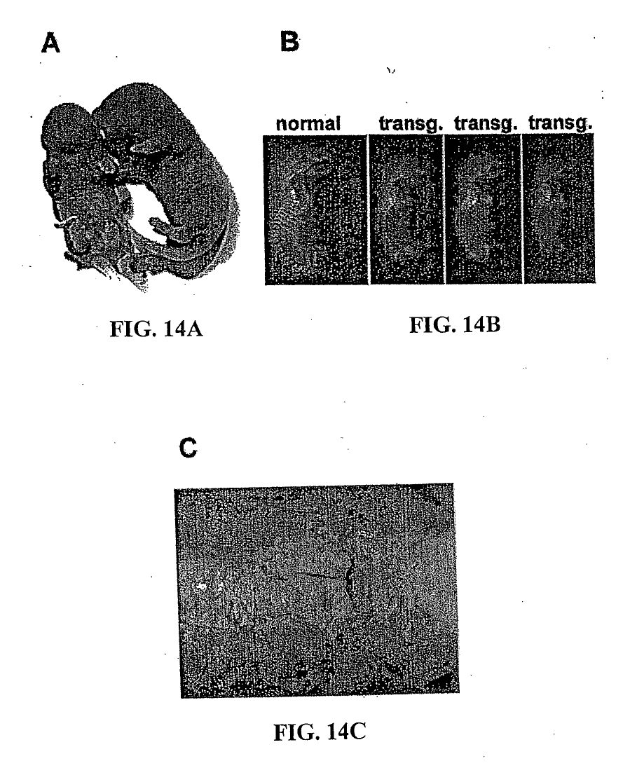

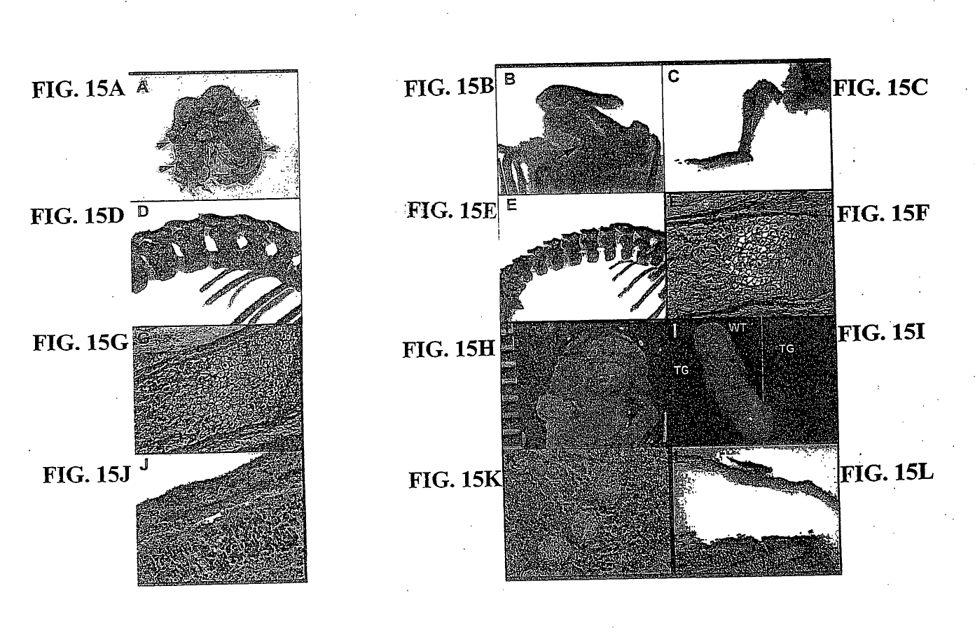

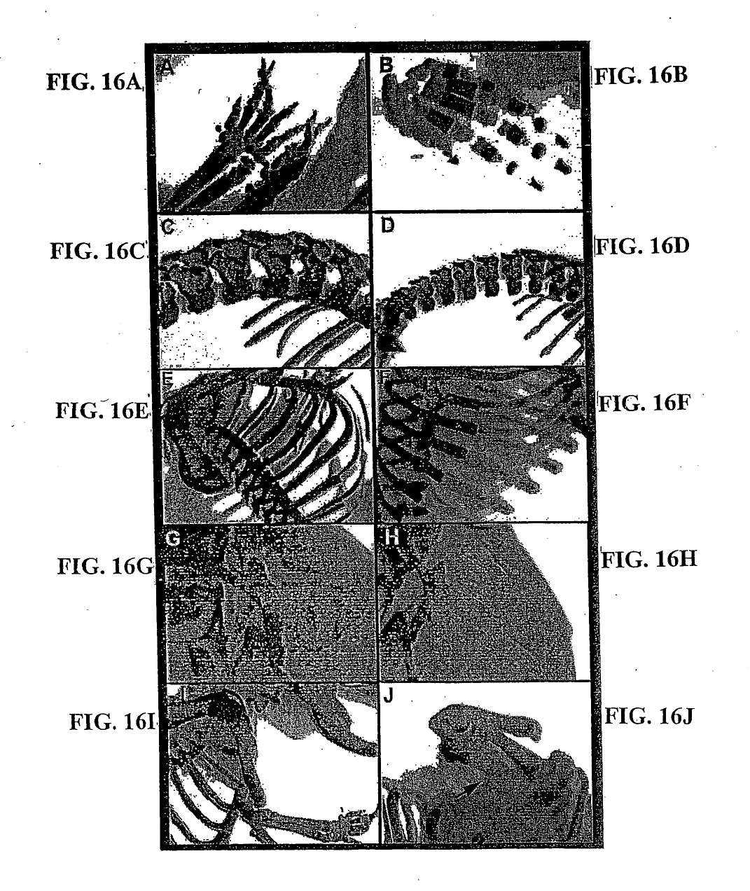

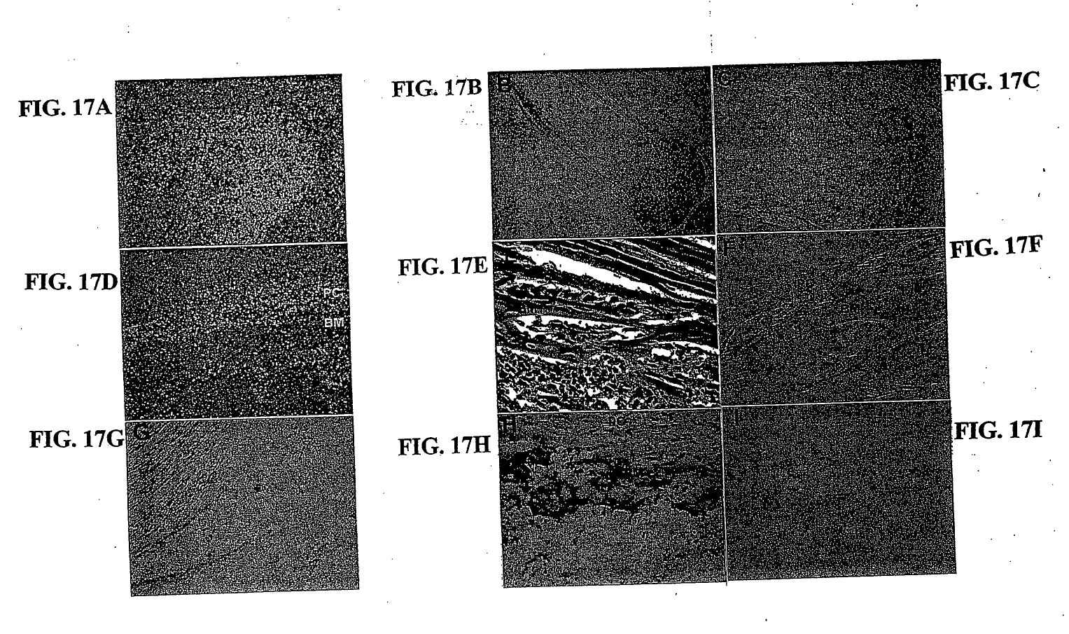

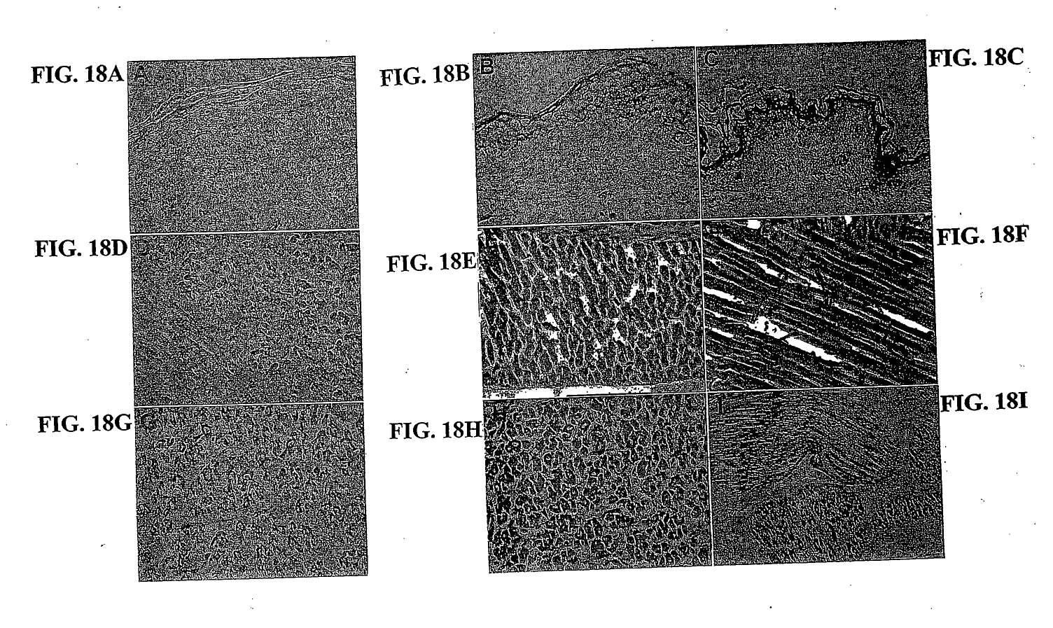

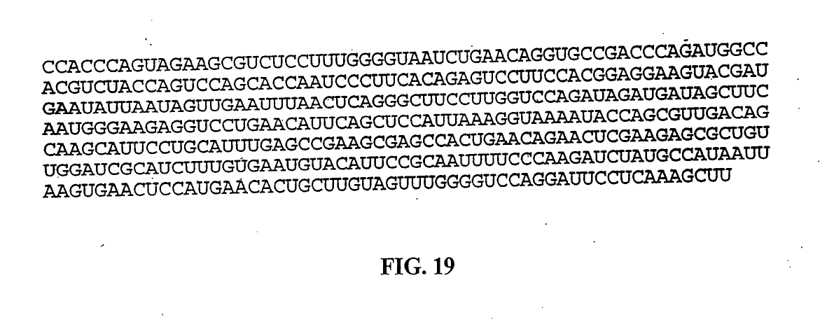

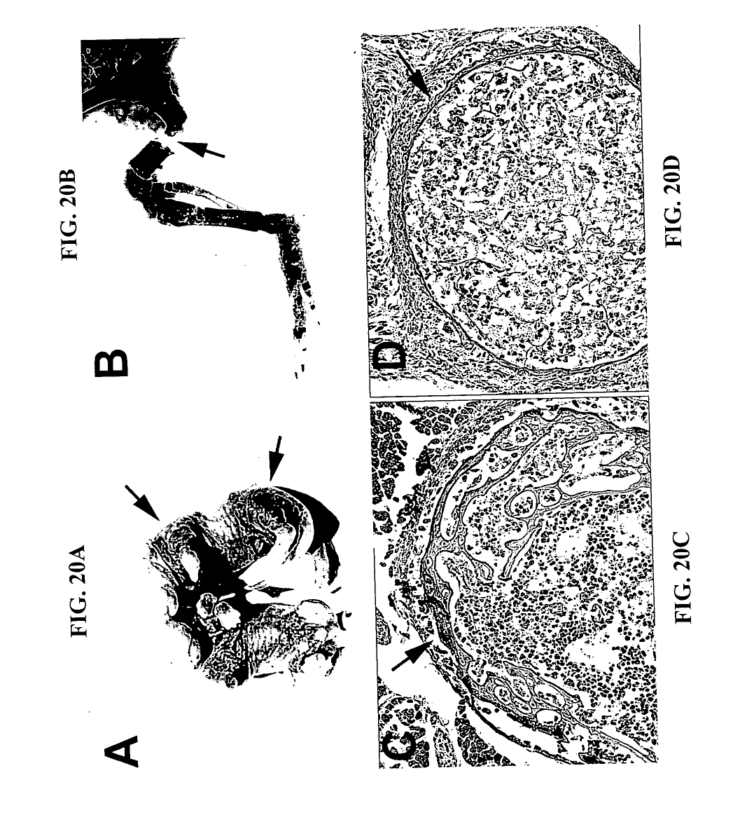

FIG. 3 is a diagram depicting the putative domains within the rat CTHRC1-short (CTHRC1s) protein. The following domains are indicated: transmembrane domain/signal peptide (amino acid residues from about 1 to 32); a CK2 phosphorylation domain (amino acid residues from about 31 to 34); an N-myristoylation domain (amino acid residues from about 69 to 74); a CK2 phosphorylation domain (amino acid residues from about 99 to 102); an N-myristoylation domain (amino acid residues from about 119 to 124); a PKC phosphorylation domain (amino acid residues from about 146 to 148); an N-myristoylation domain (amino acid residues from about 165 to 170); an N-glycosylation domain (amino acid residues from about 188 to 191); a CK2 phosphorylation domain (amino acid residues from about 197 to 200); an N-myristoylation domain (amino acid residues from about 201 to 206); an N-myristoylation domain (amino acid residues from about 205 to 210); and a CK2 phosphorylation domain (amino acid residues from about 219 to 222).

FIG. 4A is an image depicting the nucleic acid sequences for the rat (SEQ ID NO:1) and human (SEQ ID NO:3) CTHRC1 cDNA. Sequence homology between rat and human CTHRC1 cDNA is about 78% at the amino acid level. Translational start sites and stop codons are underlined. Gaps introduced into a sequence to maximize the alignment are indicated by a dash (“−”).

FIG. 4B is an image depicting a comparison of the amino acid sequences of rat (SEQ ID NO:2) and human (SEQ ID NO:4) CTHRC1. The data disclosed demonstrate that the two proteins share about 95% sequence identity. A consensus sequence is depicted between the two sequences. The “+” indicates a conserved amino acid substitution whereas “−” indicates either a gap or non-conserved amino acid substitution.

FIG. 4C is an image depicting the amino acid sequence of the long form of rat CTHRC1 (rCTHRC1_{L}) (SEQ ID NO:5), encoded by the isolated nucleic acid SEQ ID NO:1 depicted in FIG. 4A, supra.

FIG. 5A is an image of an autoradiograph depicting expression of CTHRC1 (REMODEL) protein using a rabbit reticulocyte lysate expression system. The image depicts the proteins produced by in vitro translation using the long and short forms of CTHRC1 cDNA. Using the long form of the rat CTHRC1 cDNA that contains an additional 5′ in frame AUG start codon as a template, a predominant 34 kDa protein was expressed and lesser amounts of a 30 kDa protein was detected. Only the 30 kDa protein was produced when translation was performed using the short form of CTHRC1 cDNA.

FIG. 5B is an image depicting NIH3T3 cells transfected with a myc-tagged CTHRC1 (myc-CTHRC1) expression construct. The myc-CTHRC1 fusion protein product was detected using anti-myc antibody using confocal microscopy. The image depicts that immunoreactivity was observed throughout the cytoplasm in a punctate/vesicular pattern. Nuclear counterstain was performed using propidium iodide.

FIG. 5C is an image of an immunoblot probed using rabbit antibody raised by immunizing using the carboxyterminal 15 amino acid residues of CTHRC1 (i.e., anti-CTHRC1 IgG). Cell lysates obtained from normal carotid arteries, and 1, 4, 7, 14 and 28 day balloon injured rat carotid arteries were resolved using SDS-PAGE and the proteins were transferred by Western blotting. The CTHRC1 antibody recognized a single band of approximately 34 kDa band only in the cell lysate prepared from the injured vessel but not in the normal vessel (nor. carotid).

FIG. 5D is an image of an immunoblot probed using rabbit anti-CTHRC1 gG demonstrating expression of CTHRC1 protein in various cell lines from different species as follows: NIH3T3, bovine aortic epithelium (BAE), PAC-1 (a rat smooth muscle cell line), Ar75 (a rat smooth muscle cell line), RASMC (rat aortic smooth muscle cells), 293 cells, BASMC (bovine aortic SMC), 10T½ cells, human umbilical vein endothelial cells (HUVEC), A431 cells, and human aortic SMC (HASMC).

FIG. 5E is an image of an immunoblot probed using rabbit anti-CTHRC1 IgG depicting the effect of TGF-β1 or soluble TGF-β receptor type II (sol. TGF-βRII) on CTHRC1 expression. MC3T3 cells were treated with 1 ng/ml of TGF-β1 or 100 ng/ml TGF-βRII and the cells were harvested at the times indicated in the image. The data disclosed demonstrate that TGF-β1 stimulated CTHRC1 expression while TGF-βRII inhibited CTHRC1 expression. Approximately 30 micrograms of protein were loaded per lane.

FIG. 5F is an image depicting BAE cells transfected with a myc-tagged CTHRC1 (myc-CTHRC1) expression construct. BAE were transiently transfected with a myc-tagged CTHRC1 expression construct. Expression of the transfected CTHRC1 fusion protein was detected using an anti-myc antibody and the data disclosed demonstrate that very little expression is detectable at 48 hours post-transfection. Without wishing to be bound by any particular theory, these data suggest loss of the transfected cells. BAE transfected with an unrelated protein (EP1) using the same vector as that used to prepare the myc-tagged CTHRC1 construct demonstrated higher levels of fusion protein expression 48 hours after transfection.

FIG. 6 is a series of images of Northern blots hybridized with the indicated probes. RNA was isolated from NIH3T3 cells stably transfected with a CTHRC1 expression vector and corresponding vector transfected cells. The antisense CTHRC1 (REMODELIN) transfected MC3T3 cells and corresponding vector transfected cells are the same as shown in FIG. 12. CTHRC1 overexpressing cells showed reduced levels of TGF-β1, collagen III, and biglycan while in antisense transfected cells, these levels were elevated. Osteopontin and ALP were highly upregulated in the absence of CTHRC1 expression while BMP-4 was down-regulated. An ethidium bromide stained membrane is shown as a loading control.

FIG. 7 is a set of graphs depicting luciferase activity in NIH3T3 cells transiently transfected with luciferase reporter constructs. Cbfal-dependent luciferase activity was completely inhibited in the presence of cotransfected CTHRC1. Luciferase activity under the control of the osteopontin promoter was inhibited in the presence of cotransfected CTHRC1.

FIG. 8 is an image of an immunoblot of recombinant CTHRC1 protein probed with an anti-His tag antibody.

FIG. 9A is an image depicting the resulting phenotypes in Xenopus embryos after injection of CTHRC1 mRNA at the oocyte 2 cell stage. At the 17-cell stage, embryos injected with lacZ control RNA (shown on the left side of the image) exhibited normal development while embryos injected with CTHRC1 mRNA (shown on the right side of the image) exhibited inhibition of neurectodermal cell migration.

FIG. 9B is an image depicting normal control embryos at the 34-cell stage.

FIG. 9C is an image depicting CTHRC1-injected 34-cell stage embryos. The CTHRC1 -injected embryos were smaller, distorted, and demonstrated abnormal development of the head compared with control embryos depicted in FIG. 6B.

FIG. 9D is an image depicting a CTHRC1-injected embryo exhibiting an unfused neurectoderm due to failure of the neural tissue cells to migrate.

FIG. 9E is an image depicting a CTHRC1-injected embryo exhibiting displaying the split tail phenotype common in CTHRC1-injected embryos.

FIG. 10 is an image depicting the nucleic acid sequence (SEQ ID NO:9) of a myc-tagged CTHRC1 construct.

FIG. 11 is an image of an immunoblot depicting CTHRC1 protein levels in MC3T3-E1 cells stably transfected with control vector or full length rat antisense CTHRC1 cDNA. Levels of CTHRC1 protein in antisense transfectants were undetectable while levels were normal in vector transfectants. The lower band is non-specific.

FIG. 12A is an image depicting the effect of CTHRC1 on cell adhesion and cell-cell contacts. MC3T3 cells were stably transfected with control vector and clonal populations were isolated. The data disclosed demonstrate that vector-transfected cells were of a cobblestone morphology. The image depicts a phase contrast image using 200× original magnification.

FIG. 12B is an image depicting the effect of CTHRC1 on cell adhesion and cell-cell contacts. MC3T3 cells were stably transfected with control vector and clonal populations were isolated. The data disclosed demonstrate that vector-transfected cells were of a cobblestone morphology. The image depicts a phase contrast image using 200× original magnification.

FIG. 12C is an image depicting the effect of CTHRC1 on cell adhesion and cell-cell contacts. MC3T3 cells were stably transfected with control vector and clonal populations were isolated. The data disclosed demonstrate that vector-transfected cells were of a cobblestone morphology. The image depicts a phase contrast image using 200× original magnification.

FIG. 12D is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 12E is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 12F is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 12G is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 12H is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 121 is an image depicting the effect of CTHRC1 on cell adhesion and cell-cell contacts. MC3T3 cells were stably transfected with full length rat antisense CTHRC1 cDNA and clonal populations were isolated. The data disclosed herein demonstrate that antisense CTHRC1 transfected cells exhibit a distinctly altered phenotype with less adhesion to the substratum and reduced cell-cell contacts compared with control vector transfected cells. The image further depicts the increased number in dead cells and cell debris in the antisense transfected cells. The image depicts a phase contrast image using 200× original magnification.

FIG. 13A is a graph demonstrating that CTHRC1 (REMODEL) expression is associated with and/or mediates increased cell turnover. MC3T3 cells were transfected with control vector or full-length rat antisense CTHRC1 cDNA and clonal populations were isolated. Cells were harvested at the time points indicated and cell numbers were determined. The data disclosed demonstrate that there was no increase in cell number in the antisense transfected cells compared with control cells.

FIG. 13B is a graph demonstrating that CTHRC1 (REMODEL) expression is associated with and/or mediates increased cell turnover. MC3T3 cells were transfected with control vector or full-length rat antisense CTHRC1 cDNA and clonal populations were isolated. The cells were pulsed with [³H]-thymidine for 4 hours before measuring incorporation of tritium in DNA. The cells were harvested in parallel at the time points after plating indicated and cell numbers were determined (FIG. 9A). The data disclosed demonstrate that there was increased cell turnover since there was increased [³H]-thymidine incorporation but there was no increase in cell number in the antisense transfected cells compared with control cells (FIG. 9A).

FIG. 14A is an image depicting one day old CTHRC1 transgenic mouse pups. Transgenic mice expressing CTHRC1 under the control of the cytomegalovirus (CMV) promoter/regulatory sequence were generated and a transgenic female was bred with a transgenic male giving rise to the pups depicted herein. All of the transgenic pups exhibited hemorrhaging in the hip and shoulder regions.

FIG. 14B is an image depicting one day old CTHRC1 transgenic mouse pups. Transgenic mice expressing CTHRC1 under the control of the cytomegalovirus (CMV) promoter/regulatory sequence were generated and a transgenic female was bred with a transgenic male giving rise to the pups depicted herein. X-ray examination of the skeleton identified that all transgenic mice were smaller with considerable shortening of the long bones. The image of three transgenic (“transg.”) and one normal mouse are depicted.

FIG. 14C is an image depicting one day old CTHRC1 transgenic mouse pups. Transgenic mice expressing CTHRC1 under the control of the cytomegalovirus (CMV) promoter/regulatory sequence were generated and a transgenic female was bred with a transgenic male giving rise to the pups depicted herein. Without wishing to be bound by any particular theory, similar to a spina bifida phenotype, the transgenic mice exhibited protrusion of neural tissue through the dorsal muscle layers in the thoracic area.

FIG. 15A is an image depicting one day old transgenic mouse pups expressing CTHRC1 under the control of the CMV promoter. Breeding of a transgenic female with a transgenic male gave rise to the pups depicted. The arrowheads indicate hemorrhaging in the hip and shoulder regions.

FIG. 15B is an image depicting one day old transgenic mouse pup expressing CTHRC1 under the control of the CMV promoter. Breeding of a transgenic female with a transgenic male gave rise to the pup depicted. Skeletal preparations revealed fractures of the humerus.

FIG. 15C is an image depicting one day old transgenic mouse pup expressing CTHRC1 under the control of the CMV promoter. Breeding of a transgenic female with a transgenic male gave rise to the pup depicted. Skeletal preparations revealed fractures of the femur.

FIG. 15D is an image depicting an age matched normal mouse. Alcian blue staining of skeletal preparations is shown.

FIG. 15E is an image depicting one day old transgenic mouse pup expressing CTHRC1 under the control of the CMV promoter. Breeding of a transgenic female with a transgenic male gave rise to the pup depicted. Alcian blue staining was absent from the intervertebral joints and the posterior portions of the vertebrae in CTHRC1 transgenic mice compared to normal controls (see FIG. 15D).

FIG. 15F is an image depicting an age matched normal mouse. Cortical bone matrix of the tibia is shown.

FIG. 15G is an image depicting one day old transgenic mouse pup expressing CTHRC1 under the control of the CMV promoter. Breeding of a transgenic female with a transgenic male gave rise to the pup depicted. Cortical bone matrix of the tibia (stained blue; shown in gray) was a markedly reduced in transgenics compared to controls (see FIG. 15F).

FIG. 15H is an image depicting a transgenic pup from a transgenic/wildtype cross with a more severe phenotype which died prenatally. Severe skeletal abnormalities and separation of the skin were evident.

FIG. 15I is an image depicting a transgenic pup from a transgenic/wildtype cross with a more severe phenotype which died prenatally. X-ray analysis demonstrated dwarfism, a dramatic reduction in bone density, and malformations of all bones. (WT=wildtype; TG=CTHRC1 transgenic).

FIG. 15J is an image depicting a transgenic pup from a transgenic/wildtype cross with a more severe phenotype which died prenatally. Histologically, there was a considerable reduction in bone matrix (stained blue; shown in gray) in all bones including the skull.

FIG. 15K is an image depicting a transgenic pup from a transgenic/wildtype cross with a more severe phenotype which died prenatally. Ribs had very little bone matrix.

FIG. 15L is an image depicting a transgenic pup from a transgenic/wildtype cross with a more severe phenotype which died prenatally. Skin sections revealed separation of the epidermis from the dermis.

FIG. 16A is an image depicting skeletal preparation made from one day old non-transgenic pups otherwise identical to CTHRC1 transgenic pups. Mineralized bone appears pink in color (darker gray) and cartilage appears blue (lighter gray).

FIG. 16B is an image depicting skeletal preparation made from one day old CTHRC1 transgenic pups. Mineralized bone appears pink in color (darker gray) and cartilage appears blue (lighter gray). The image depicts reduced cartilage formation in all bones, including the distal phalanges of the feet when compared with normal, non-transgenic pups (FIG. 11A).

FIG. 16C is an image depicting skeletal preparation made from one day old non-transgenic pups otherwise identical to CTHRC1 transgenic pups. Mineralized bone appears pink in color (darker gray) and cartilage appears blue (lighter gray).

FIG. 16D is an image depicting skeletal preparation made from one day old CTHRC1 transgenic pups. Mineralized bone appears pink in color (darker gray) and cartilage appears blue (lighter gray). The image depicts that cartilage was absent from the intervertebral joints and the posterior portions of the vertebra when compared with normal, non-transgenic pups (FIG. 11C).

FIG. 16E is an image depicting skeletal preparation made from one day old non-transgenic pups otherwise identical to CTHRC1 transgenic pups. Mineralized bone appears pink in color (darker gray) and cartilage appears blue (lighter gray).

FIG. 16F is an image depicting skeletal preparation made from one day old CTHRC1 transgenic pups. Mineralized bone appears pink in color (darker gray) and cartilage appears blue (lighter gray). The image depicts that the anterior portions of the ribs, particularly the more caudal ones, exhibited a marked decrease in cartilage content when compared with normal, non-transgenic pups (FIG. 11E).

FIG. 16G is an image depicting skeletal preparation made from one day old non-transgenic pups otherwise identical to CTHRC1 transgenic pups. Mineralized bone appears pink in color (darker gray) and cartilage appears blue (lighter gray).

FIG. 16H is an image depicting skeletal preparation made from one day old CTHRC1 transgenic pups. Mineralized bone appears pink in color (darker gray) and cartilage appears blue (lighter gray). The image depicts that the transgenic pups exhibited decreased bone density, particularly in the flat bones of the skull which had a transparent appearance, when compared with normal, non-transgenic pups (FIG. 11G).

FIG. 16I is an image depicting skeletal preparation made from one day old non-transgenic pups otherwise identical to CTHRC1 transgenic pups. Mineralized bone appears pink in color (darker gray) and cartilage appears blue (lighter gray).

FIG. 16J is an image depicting skeletal preparation made from one day old CTHRC1 transgenic pups. Mineralized bone appears pink in color (darker gray) and cartilage appears blue (lighter gray). The image depicts that the transgenic pups exhibited decreased bone density compared with normal, non-transgenic pups (FIG. 11I). The data disclosed demonstrate that decreased bone density is associated with fragility leading to multiple fractures such as a fractured humerus (arrow), which explain the hemorrhaging observed in upper and lower limbs (FIG. 10A).

FIG. 17A is an image depicting endogenous CTHRC1 expression in normal one day old mouse pups. In situ hybridization with antisense CTHRC1 RNA shows mRNA expression in the cartilage of the humerus head.

FIG. 17B is an image depicting endogenous CTHRC1 expression in normal one day old mouse pups. Immunostaining with anti-CTHRC1 IgG on a longitudinal section of the tibia.

FIG. 17C is an image depicting endogenous CTHRC1 expression in normal one day old mouse pups. Higher magnification of a tibia head stained for CTHRC1.

FIG. 17D is an image depicting endogenous CTHRC1 expression in normal one day old mouse pups. CTHRC1 mRNA is expressed in periosteal cells (PC) of the femur.

FIG. 17E is an image depicting endogenous CTHRC1 expression in normal one day old mouse pups. Masson's Trichrome stained section depicting cortical bone of the tibia.

FIG. 17F is an image depicting endogenous CTHRC1 expression in normal one day old mouse pups. Similar section as shown in FIG. 17E depicting some CTHRC1 immunoreactivity in PC and osteoblasts (arrows) but not in osteocytes of the bone matrix (BM).

FIG. 17G is an image depicting expression of the CTHRC1-myc transgene in mice. Immunostaining with the anti-myc antibody was performed on a section of the arm depicting the proximal radius.

FIG. 17H is an image depicting expression of the CTHRC1-myc transgene in mice. High levels of the transgene proteins are expressed by osteocytes (arrowheads) and lower levels are expressed in osteoblasts.

FIG. 17I is an image depicting expression of the CTHRC1 -myc transgene in mice. Preimmune IgG showed little background on section of tibia cartilage.

FIG. 18A is an image depicting one day old normal mice. Endogenous CTHRC1 protein was not detectable in normal skin by immunostaining with anti-CTHRC1 IgG.

FIG. 18B is an image depicting one day old CTHRC1 transgenic mice. High levels of the CTHRC1 transgene were detected in the epidermis with anti-myc antibody and lower levels were detected in the dermis.

FIG. 18C is an image depicting one day old CTHRC1 transgenic mice. The connective tissue of the dermis appeared very loose (Trichrome stain).

FIG. 18D is an image depicting one day old normal mice. Skeletal muscle in normal mice showed endogenous CTHRC1 expression with anti-CTHRC1 IgG immunostaining.

FIG. 18E is an image depicting cross-sectioned skeletal muscle fibers from one day old normal mice.

FIG. 18F is an image depicting longitudinally-sectioned skeletal muscle fibers from one day old normal mice.

FIG. 18G is an image depicting cross-sectioned skeletal muscle fibers from one day old CTHRC1 transgenic mice. The transgene specific CTHRC1-myc antibody demonstrated high levels of immunoreactivity in skeletal muscle of transgenic mice and many of the fiber bundles were hollow (arrowheads).

FIG. 18H is an image depicting cross-sectioned skeletal muscle fibers from one day old CTHRC1 transgenic mice. Muscle fibers of transgenic mice formed circular structures (arrowheads).

FIG. 18I is an image depicting cross-sectioned skeletal muscle fibers from one day old CTHRC1 transgenic mice. Muscle fibers of transgenic mice were less densely packed.

FIG. 19 is an image depicting the sequence of an isolated CTHRC1 antisense ribonucleic acid (SEQ ID NO:6) complementary to a portion of a nucleic acid encoding CTHRC1.

FIG. 20, comprising FIGS. 20A through 20D depict the effect of overexpression of CTHRC1 in transgenic mice. FIG. 20A is an image depicting newborn CTHRC1 transgenic mice hemorrhaging in hip and shoulder region (arrows). FIG. 20B is an image depicting a bone preparation of newborn CTHRC1 transgenic mouse indicating a fractured femur (arrow). FIG. 20C is an image of a photomicrograph of a cross-section of femur from newborn wildtype mouse demonstrating abundant cortical bone matrix (arrow) and trabecular bone. FIG. 20D is an image of a photomicrograph of a cross-section of femur from a CTHRC1 transgenic newborn mouse demonstrating reduced cortical bone matrix (arrow) and lack of trabecular bone matrix.

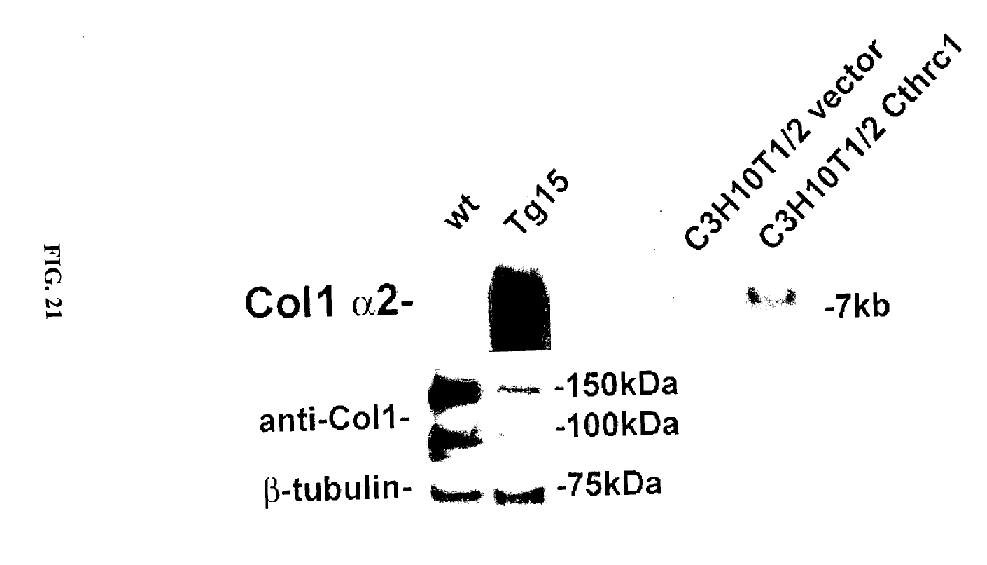

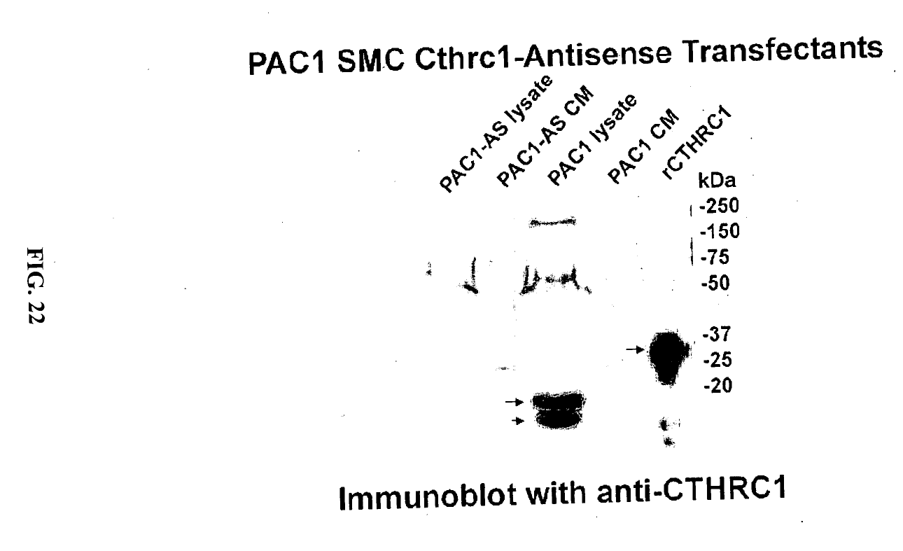

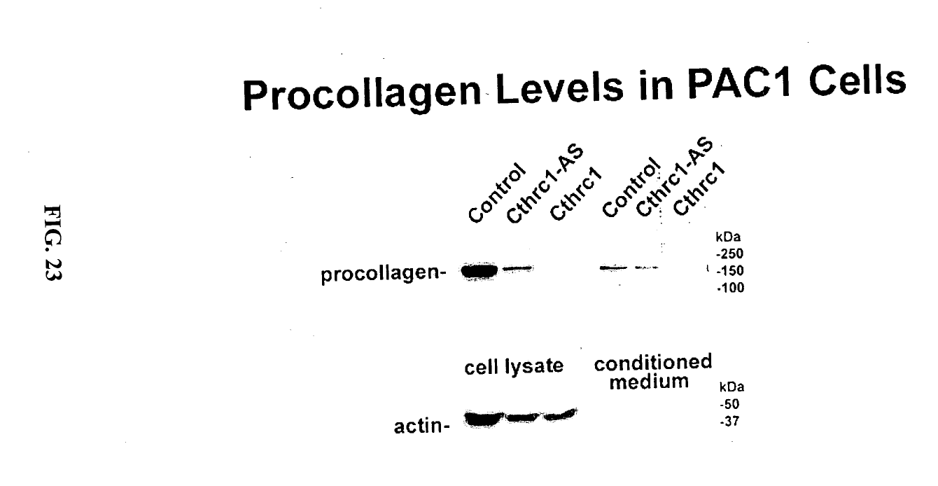

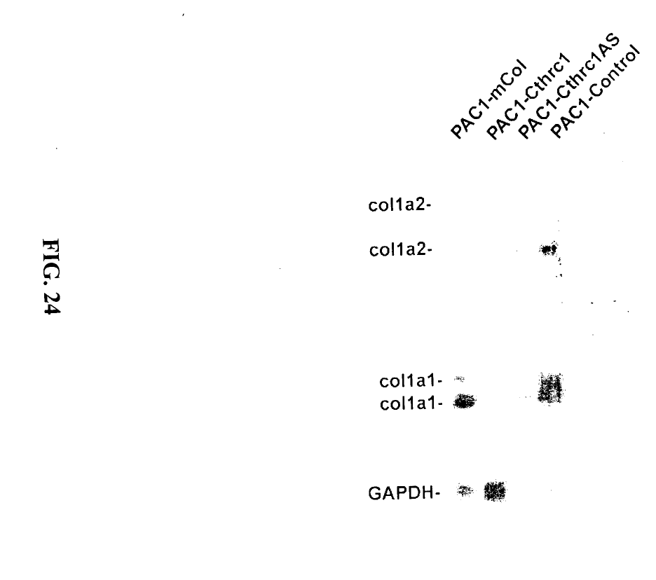

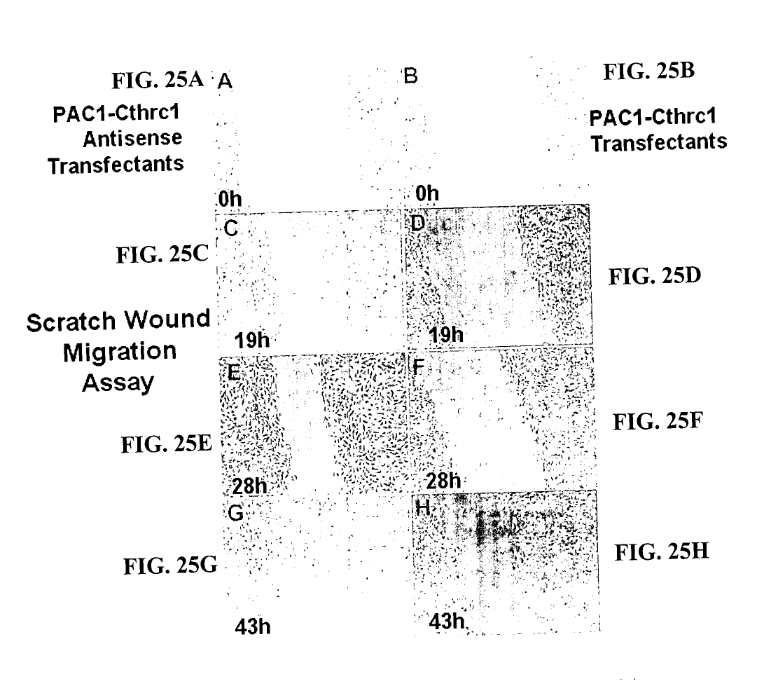

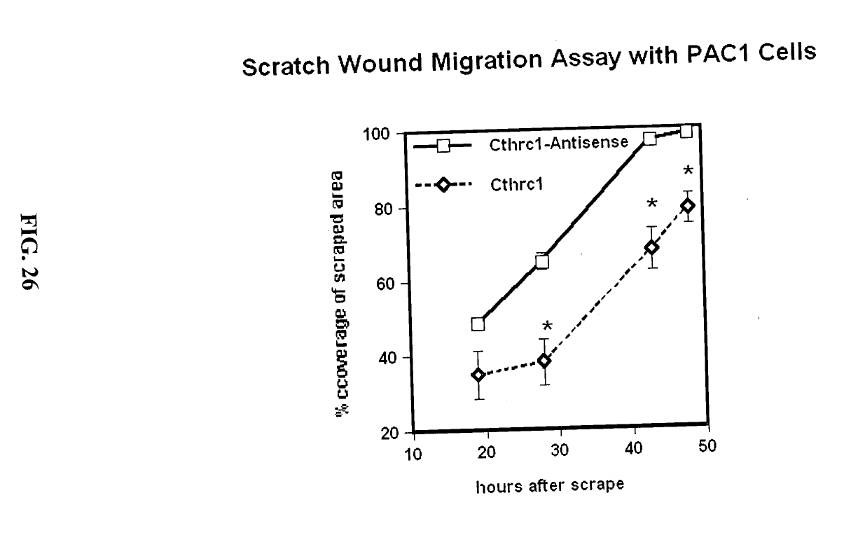

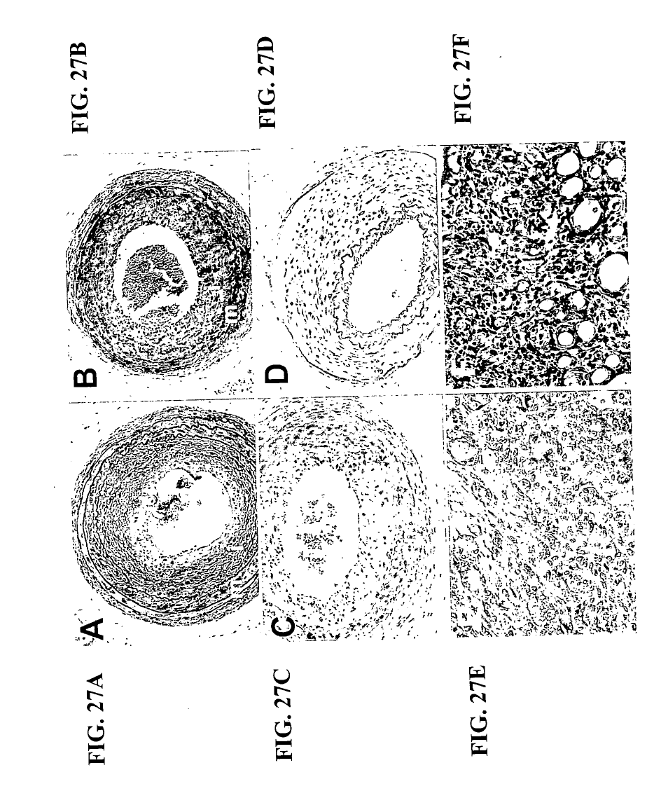

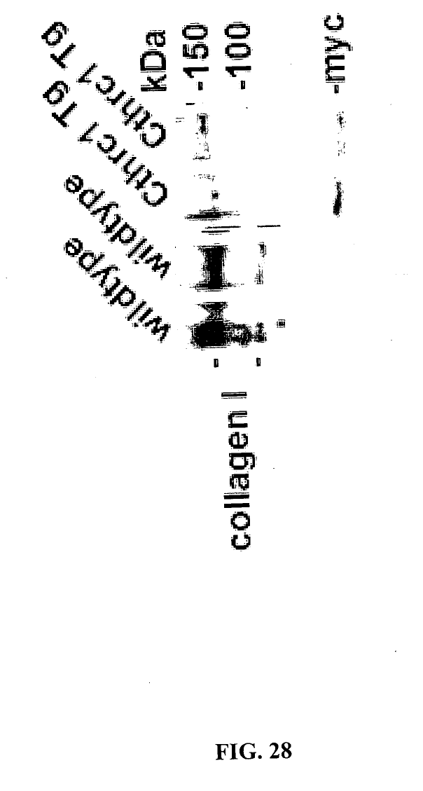

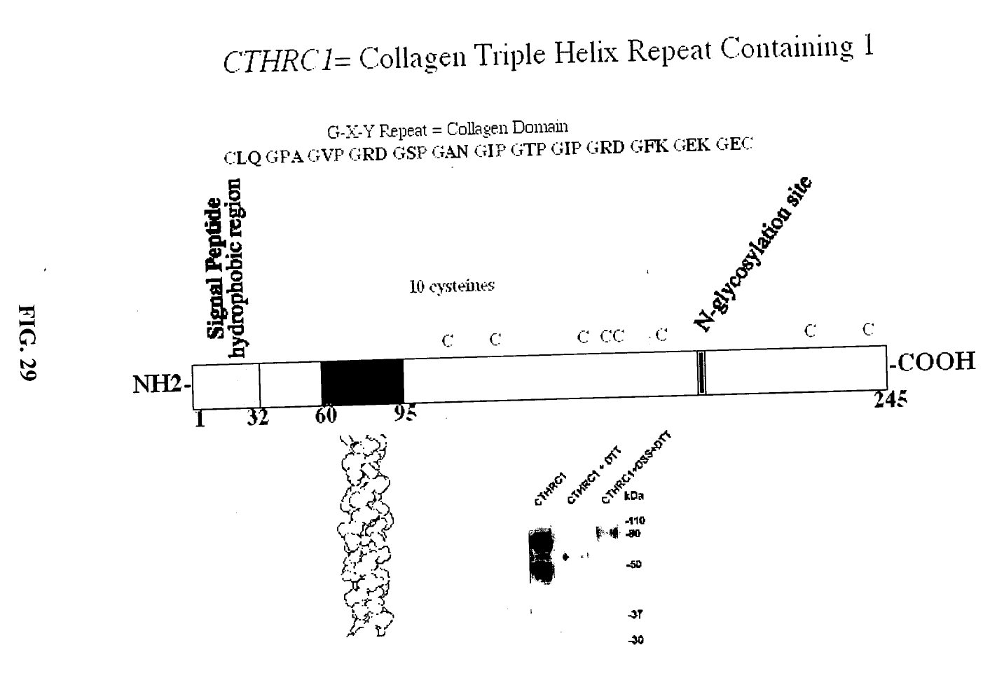

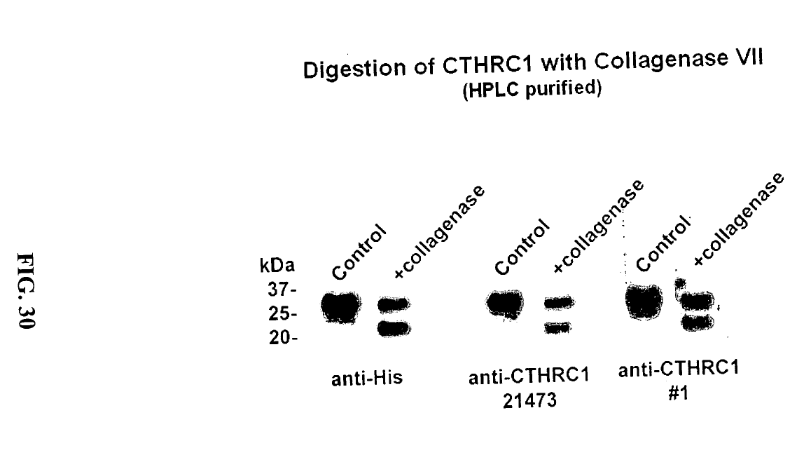

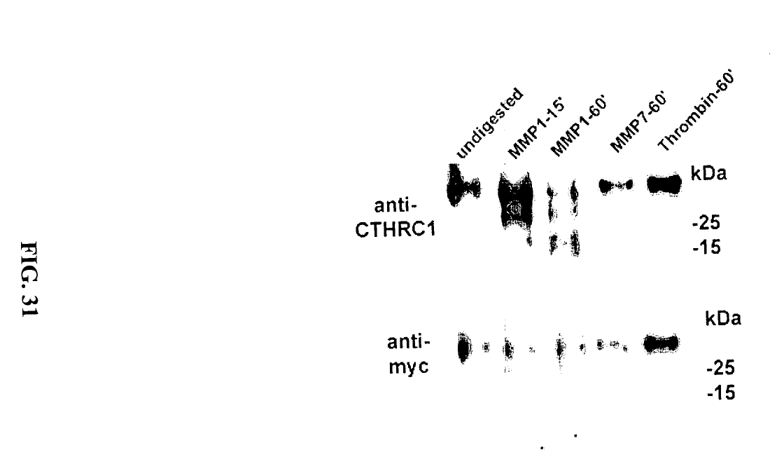

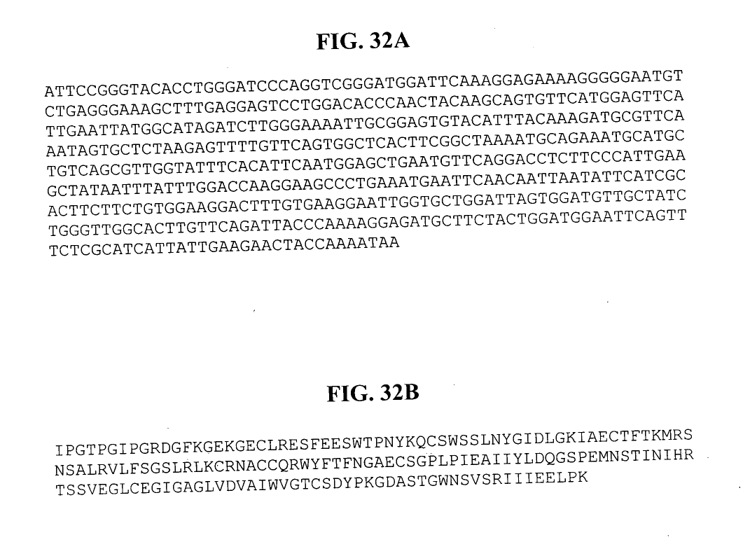

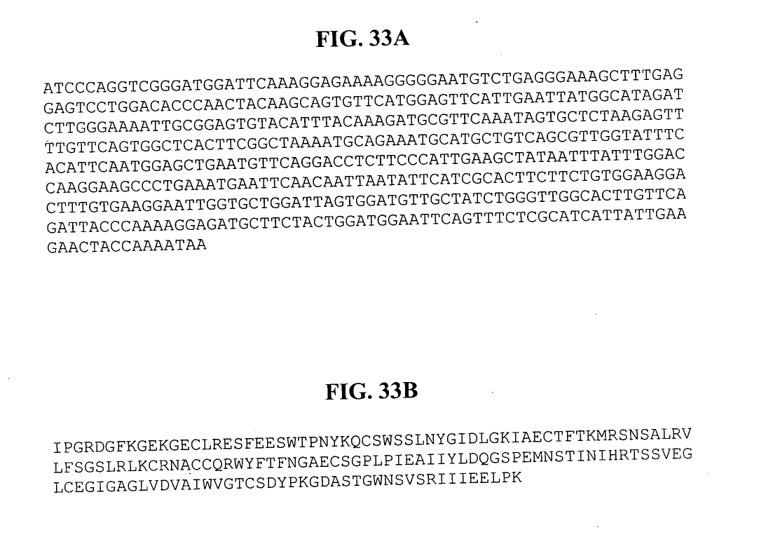

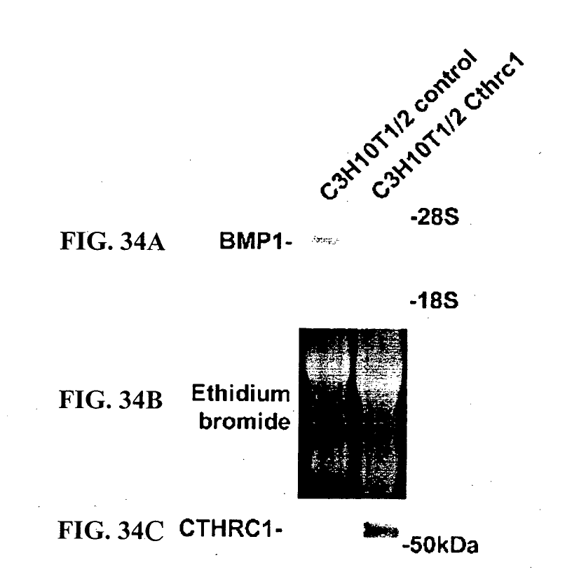

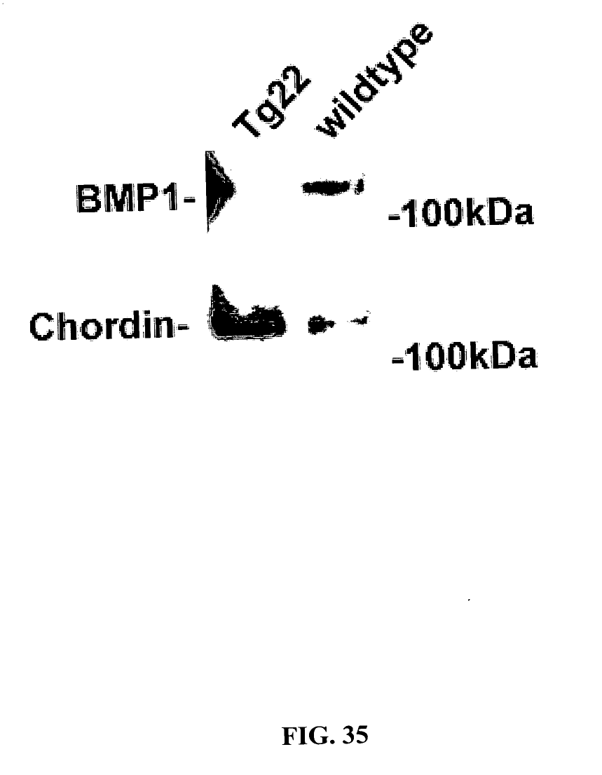

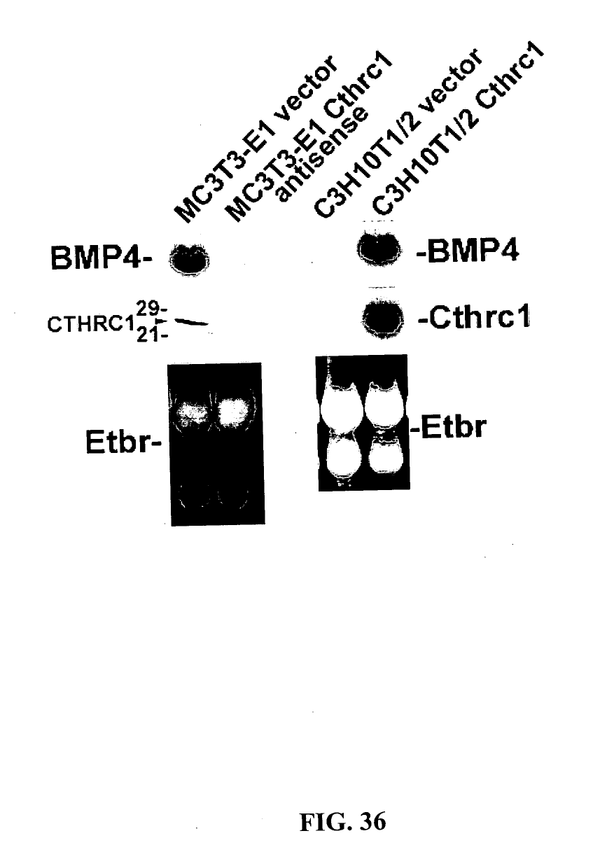

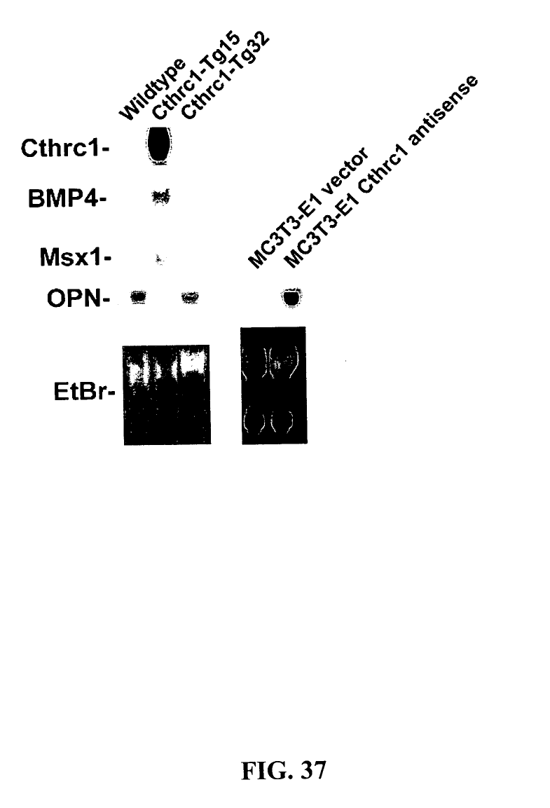

FIG. 21 is an image depicting an immunoblot (left side of figure) of skull bones from transgenic mouse pups overexpressing CTHRC1 (lane labeled “Tgl5”) and corresponding wildtype mice (“wt”) (left top panel). An image of a Northern blot analysis also performed on RNA isolated from C3H10T½ cells transformed with control vector and CTHRC1 expression vector (right panel of figure) is depicted on the right side of the figure. Northern blot analysis with a rat collagen I alpha2 chain probe demonstrated increased levels of collagen I mRNA in CTHRC1 transgenic skull bones as well as C3H20T½ cells overexpressing CTHRC1. Immunoblotting with a rabbit polyclonal anti-collagen type I antibody (Research Diagnostics) demonstrated reduced collagen type I protein levels in transgenic skull bones (left center panel at about 100 kDa and 150 kDa). Immunoblotting with a monoclonal anti-β-tubulin antibody (Sigma) of the same skull bone lysates demonstrates equal loading of the samples (left bottom panel at about 75 kDa).

FIG. 22 is an image depicting an immunoblot analysis using anti-CTHRC1 antibody of PAC1 cell lysates (“PAC1 lysate”) and conditioned media (“PAC1 CM”) obtained from CTHRC1 anti-sense transfectants (“PAC1-AS lysate” and “PAC1-AS CM”) and control cells (PAC1). Full-length recombinant CTHRC1 (“rCTHRC1”) indicated in the far right lane (arrow). Note the CTHRC1 immunoreactive bands in the control cells (arrows) which are absent in the antisense transfected cells. Also note that endogenous CTHRC1 in PAC1 shows two fragments of lower molecular weight (arrows) compared to rCTHRC1 from CHO cells (arrow).

FIG. 23 is an image depicting immunoblot analysis using anti-procollagen antibody of cell lysates and conditioned media. Cell lysates and conditioned media from untransfected (“Control”), CTHRC1 antisense (“Cthrc1-AS”), and CTHRC1 overexpressing PAC1 cells (“Cthrc1”) are shown. For normalization of cellular protein loading, the same amount of cell lysate was also immunoblotted with an anti-actin antibody (left lanes of blot). Note the decrease/absence of detectable procollagen immunoreactivity in the CTHRC1 overexpressing cells (both cell lysate and in conditioned medium).

FIG. 24 is an image depicting a Northern blot analysis demonstrating that over-expression of CTHRC1 in a cell inhibits collagen 1 mRNA expression. Smooth muscle PAC-1 cells were stably transfected with one of the following constructs: wildtype CTHRC1 (PAC1-Cthrc1); a mutant Cthrc1 (PAC1-mCol) wherein the collagen domain of CTHRC1 (comprising the amino acid sequence GPAGVPGRDGSPGANGIPGTPGIPGRDGFKGEKG [SEQ ID NO:14] encoded by the nucleic acid sequence ggaccagcaggagttcctggtcgcgatgggagccctggggccaatggcattcctggcacaccgggaatcccaggtcg ggatggattcaaaggagagaaaggg [SEQ ID NO:15]) was replaced with a collagen domain of equal length obtained from mouse collagen 1 alpha1 (col1a1) (comprising amino acid sequence GSPGTAGARGNDGAVGAAGPPGPTGPTGPPGFGF [SEQ ID NO:16], encoded by nucleic acid sequence ggatcccctggcactgctggtgctc gcggtaacga tggtgctgtt ggtgctgctg gaccccctgg tcccaccggccccactggcc ctcctggcttcggattc [SEQ ID NO:17]); and antisense Cthrc1 (PAC1-Cthrc1AS) described elsewhere herein. Non-transfected PAC1 cells were also used as controls (PAC1-Control). Northern blots of mRNA derived from each cell line were probed with radiolabeled mouse collagen 1 alpha1 (col1a1), rat collagen 1 alpha2 (col1a2), as well as with GAPDH as an internal loading control. Two transcripts detectable for both collagen 1 alpha1 and collagen 1 alpha2. The data disclosed herein demonstrate that overexpression of wildtype Cthrc1, but not expression of mutant CHTRC1 or antisense CTHRC1, was associated with profound down-regulation of all collagen 1 mRNA transcripts.

FIG. 25, comprising FIGS. 25A through 25H, is an image of a photomicrograph depicting a scratch wound migration assay with CTHRC1 transfected PAC1 cells. Note the delayed closure of the scraped area in the CTHRC1 overexpressing cells (FIGS. 25D, F, and H) compared with untransfected control PAC1 cells (FIGS. 25C, E, and G) over time (from about 0 hours to about 43 hours after scrape).

FIG. 26 is a graph depicting quantification of scratch wound migration assay.

FIG. 27, comprising FIGS. 27A through 27E, depicts fibrosis in carotid arteries from wildtype mice (FIGS. 27A and 27B) compared with CTHRC1 transgenic mice (FIGS. 27C and 27D) assessed by Trichrome staining of sections obtained 2 weeks after vessel ligation. Note the markedly reduced amount of collagenous matrix deposition (less blue staining as indicated by gray shading) in CTHRC1 transgenic mice (FIGS. 27C-D). FIG. 27E is an image of a photomicrograph depicting the effects of CTHRC1 on renal fibrosis evaluated in an art-recognized obstructive uropathy model performed in wildtype mice compared with CTHRC1 transgenic mice (FIG. 27F). The degree of fibrosis was assessed by Trichrome staining which showed reduced collagen deposition in CTHRC1 transgenic mice (FIG. 27F) compared to controls (FIG. 27E) depicted by lighter gray shading.

FIG. 28 is an image depicting an immunoblot analysis of lysates from kidneys 4 days after ureter obstruction probed with anti-collagen type I (Calbiochem) and anti-myc antibody (Zymed). Kidneys from wildtype mice demonstrated higher levels of collagen protein production compared with CTHRC1 transgenic (“Cthrc1 Tg”) mice. Expression of the transgene is demonstrated by presence of the myc-tagged CTHRC1 protein (“myc”).

FIG. 29, without wishing to be bound by any particular theory, depicts a schematic representation of the CTHRC1 protein indicating, among other things, the various domains. The collagen domain (amino acids 60-95) is indicated. Note that there are 2 G-X—Y repeats in which a G (glycine) is followed by an I (isoleucine) as indicated by the amino acid sequence depicted above the diagram. In the bottom portion of the figure is an image depicting an immunoblot probed with anti-CTHRC1 antibody. Under non-reducing conditions recombinant CTHRC1 (lane 1) migrates at a molecular weights consistent with a dimer and trimer. Cross-linked CTHRC1 run under reducing conditions (lane 3, CTHRC1+DSS+DTT) migrates at a molecular weight consistent with a trimer.

FIG. 30 is an image depicting an immunoblot of His-tagged CTHRC1 expressed in Chinese hamster ovary cells (CHO) probed with anti-His and anti-CTHRC1 antibodies (21473 and #1) following digestions with highly purified collagenase (VII, Sigma). Since the His tag is located at the C terminus of CTHRC1, the data indicate that collagenase cleaves CTHRC1 near the N terminus.

FIG. 31 is an image depicting an immunoblot of myc-tagged CHO cell-expressed CTHRC1 probed with anti-CTHRC1 antibody (top portion of panel) and the same blot was also probed with anti-myc antibody (bottom portion of panel) following digestion with matrix metalloprotease 1 (MMP-1) (15 and 60 minutes digest), MMP-7 and thrombin. Note the cleavage of CTHRC1 by MMP-1, but no detectable cleavage by MMP-7 or thrombin. Since the myc tag is located at the C terminus, the data indicate that MMP1 cleaves CTHRC1 near the N terminus.

FIG. 32A depicts the nucleic acid sequence corresponding to human CTHRC1 cleaved by MMP-1 to produce a longer cleavage fragment (cCTHRC1_{L}) (SEQ ID NO:10).

FIG. 32B depicts the amino acid sequence of human CTHRC1 cleaved by MMP-1 to produce a longer cleavage fragment (cCTHRC1_{L}) (SEQ ID NO:11).

FIG. 33A depicts the nucleic acid sequence corresponding to the amino acid sequence of human CTHRC1 cleaved by MMP-1 to produce a shorter cleavage fragment (cCTHRC1_{S}) (SEQ ID NO:12).

FIG. 33B depicts the amino acid sequence of human CTHRC1 cleaved by MMP-1 to produce a shorter cleavage fragment (cCTHRC1_{S}) (SEQ ID NO:13).

FIG. 34, comprising FIGS. 34A through 34C, is an image illustrating the results of an examination of the expression of BMP1 in C3H10T½ cells transfected with an empty vector (lane 1) or with a Cthrc1 expression construct (lane 2). Northern blot analysis demonstrated that BMP1 levels are reduced in Cthrc1 overexpressing C3H10T½ cells (FIG. 34A). The ethidium bromide stained gel indicates equal loading of total RNA (FIG. 34B). Immunoblotting of C3H10T½ cell lysates under non-reducing conditions with anti-CTHRC1 antibody shows increased levels of the CTHRC1 in Cthrc1 transfected cells (FIG. 34C).

FIG. 35 is an image of an immunoblot depicting lysates prepared from newborn mouse skin. Lysates were immunoblotted with anti-BMP1 and anti-Chordin antibodies. Skin from Cthrc1 transgenic mice (lane labeled ‘Tg22’) demonstrated reduced amounts of BMP1 and increased levels Chordin. Equal amounts of protein (100 μg) were loaded in each lane.

FIG. 36 comprises a series of images depicting effects of decreasing or increasing CTHRC1 protein and/or CTHRC1 mRNA expression. Stable antisense CTHRC1 mRNA expressing MC3T3-E1 and CTHRC1 mRNA overexpressing C3H10T½ cell lines were established. The decrease in CTHRC1 protein in CTHRC1 antisense transfectants was verified by CTHRC1 immunoblotting (left center panel). The loss of CTHRC1 expression was associated by a reduction in BMP4 mRNA levels in the MC3T3-E1 CTHRC1 antisense cells as depicted in the top left panel of FIG. 36. Overexpression of CTHRC1 mRNA (center right panel of FIG. 36) in C3H10T½ cells coincided with an induction of BMP4 mRNA expression (top right panel). The bottom panels of FIG. 36 depict ethidium bromide stained gels demonstrating equal loadings of total RNA in each gel lane.

FIG. 37 comprises a series of images comparing CTHRC1, BMP4, and osteopontin expression. Northern blot analysis of RNA extracted from wildtype mouse skull bones and from skull bones obtained from CTHRC1 overexpressing transgenic mouse lines Tg15 and Tg32 demonstrates that strong increase in CTHRC1 mRNA expression in line Tg15 is associated with an increase in BMP4 and Msx1 expression, whereas osteopontin (OPN) mRNA levels were reduced. FIG. 37 also illustrates that the inverse correlation of CTHRC1 expression and osteopontin expression in MC3T3-E1 cells expressing antisense CTHRC1. That is, the data demonstrate that OPN expression is increased in MC3T3-E1 CTHRC1 antisense cells where CTHRC1 expression is decreased (as shown in FIG. 36).

### DETAILED DESCRIPTION OF THE INVENTION

The invention relates to the discovery of a novel nucleic acid encoding a mammalian adventitia-inducible bone expressed molecule termed CTHRC1 (collagen triple helix repeat containing 1), previously referred to as REMODELIN, REMODEL and/or adventitia induced bone expressed molecule (AIBE), and the proteins encoded thereby. The data disclosed herein demonstrate that CTHRC1 plays a role in, inter alia, arterial restenosis mediated by or associated with adventitial fibrosis. As described more fully below, CTHRC1 also plays a role in bone and cartilage formation. Identification of CTHRC1 has important implications in the development of therapeutics and diagnostics for, among other things, adventitial fibrosis, arterial restenosis, constrictive remodeling, and restenosis due to wound healing. More specifically, nucleic acids encoding CTHRC1 have been isolated in both rat and human. These sequences are provided herein, and have no significant homology to any known cDNA sequence.

The data disclosed herein demonstrate that expression of CTHRC1 is induced by vessel injury in mammals. That is, CTHRC1 was expressed in balloon-injured rat carotid arteries but not in normal, uninjured vessels. Furthermore, CTHRC1 was expressed selectively in the adventitia of the injured vessel, and was not expressed in the neointima or in the adventitia of normal vessels. Moreover, CTHRC1 expression was induced by TGF-β. This is important since proliferative events occurring in the adventitia contribute to vascular remodeling and restenosis in response to vascular injury and recent data demonstrate that TGF-β is a factor in this adventitial remodeling process. Thus, these data further indicate that CTHRC1 plays a role in cell proliferation and/or migration associated with vessel injury and restenosis due to constrictive remodeling.

The data disclosed herein also demonstrate that CTHRC1 plays an important role in development of bone during mammalian embryogenesis. CTHRC1 is normally expressed during mouse embryogenesis, but expression is localized to developing bone. However, in the adult mouse, CTHRC1 expression is virtually undetectable, expressing at very low levels in the adult brain and lung tissue.

Additionally, the data disclosed herein demonstrate that in studies using frog embryos, CTHRC1 also plays a role in cell proliferation and/or migration in that expression of CTHRC1 in frog embryos resulted in inhibition blastopore closure, failure of closure of the neural folds, formation of a split tail, and other developmental abnormalities. The CTHRC1-injected embryos also presented with decreased size and distortion and abnormal development of the head.

Injection of CTHRC1 mRNA into frog embryos inhibited FGF-induced mesoderm formation. That is, animal caps from CTHRC1-injected embryos incubated with FGF-1 resembled animal caps incubated in the absence of FGF-1. Indeed, the data disclosed herein demonstrate that the phenotype observed in frog embryos injected with CTHRC1 mRNA is similar to that of embryos injected from mRNA for dominant-negative FGF receptor constructs. These results, in addition to the induction of CTHRC1 by TGF-β, further indicate that CTHRC1 is an important factor in cell proliferation, migration, or both.

Additionally, over-expression of CTHRC1 in transgenic mice gave rise to spina bifida-like spinal defects. The transgenic mouse pups exhibited altered bone density and bone growth further indicating that CTHRC1 plays an important role in embryogenesis, including, but not limited to, a role in bone growth and dorsal closure.

The data disclosed herein also demonstrate that CTHRC1 is localized in the cell membrane via five potential N-myristoylation sites. Without wishing to be bound by any particular theory, these myristoylation sites may serve to anchor CTHRC1 protein in the cell membrane. This would indicate that CTHRC1 is not a secreted protein, but rather, it is associated with the cell in mediating its effect(s).

Further, the data disclosed herein demonstrate for the first time that expression of CTHRC1 mediates a surprising detectable decrease in collagen matrix production and, thus, administration of CTHRC1 provides important novel therapeutics for treatment and prevention of disease, disorder or condition mediated by, or associated with, collage matrix production.

In sum, the data disclosed herein demonstrate that CTHRC1 plays a role in cell proliferation and/or migration and is involved in cellular signaling. Furthermore, the data demonstrate that CTHRC1 likely plays a role in adventitial fibrosis, constrictive remodeling and arterial restenosis, mediated by, among other things, smooth muscle cell proliferation. Therefore, the instant invention provides an in vitro model for the study of the function and role(s) of CTHRC1 in arterial remodeling, adventitial fibrosis, and restenosis in vessels, as well as potential therapeutics and diagnostics for treatment of diseases, disorders or conditions associated with adventitial fibrosis, arterial restenosis, bone density and bone growth.

Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

By the term “abnormal expression of CTHRC1,” as used herein, is meant that the level of expression of a CTHRC1 molecule (e.g., rat CTHRC1_{S}, rat CTHRC1_{L}, human CTHRC1) in a cell is detectably higher or lower than the level of expression of CTHRC1 in an otherwise identical cell where the otherwise identical cell is obtained from normal tissue that does not exhibit any detectable disease, disorder or condition associated with or mediated by expression of CTHRC1, such as, but not limited to, adventitial remodeling, adventitial fibrosis, arterial restenosis, constrictive remodeling, bone growth, bone fracture healing, wound healing in any tissue, and the like.

As used herein, the term “adjacent” is used to refer to nucleotide sequences which are directly attached to one another, having no intervening nucleotides. By way of example, the pentanucleotide 5′-AAAAA-3′ is adjacent the trinucleotide 5′-TTT-3′ when the two are connected thus: 5′-AAAAATTT-3′ or 5′-TTTAAAAA-3′, but not when the two are connected thus: 5′-AAAAACTTT-3′.

As used herein, amino acids are represented by the full name thereof, by the three letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

By the term “adventitial fibrosis,” as used herein, is meant the extensive detectable fibrous (connective) tissue formation in the outer layer (i.e., adventitia) of a blood vessel. Adventitial fibrosis is associated with abundant deposition of extracellular matrix and proliferation of myofibroblasts and fibroblasts.

As used herein, to “alleviate” a disease, disorder or condition means reducing the severity of one or more symptoms of the disease, disorder or condition. This can include, but is not limited to, increasing the level of CTHRC1 expressed in a cell or tissue (e.g., smooth muscle cell, lung tissue, an artery), reducing the level of cell proliferation and/or migration, reducing the production of collagen matrix, affecting wound healing, affecting granulation tissue formation, affecting bone growth and/or fracture healing, reducing constrictive remodeling, arterial restenosis and/or adventitial fibrosis, inhibiting premature calcification/ossification, inhibiting calcification of an implant (e.g. a heart valve), reducing or increasing the level of CTHRC1 in a patient, compared with the level of CTHRC1 in the patient prior to or in the absence of the method of treatment, and the like.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, a bronchoscope, a nebulizer, and the like, for administering the CTHRC1 nucleic acid, protein, and/or composition of the invention to a mammal.

“Arterial restenosis,” as that term is used herein, means the detectable re-narrowing of an artery in response to a vascular intervention aimed at dilating a stenosed (i.e., narrowed) artery compared with the artery immediately following such intervention.

“Biological sample,” as that term is used herein, means a sample obtained from an animal that can be used to assess the level of expression of a CTHRC1, the level of CTHRC1 protein present, or both. Such a sample includes, but is not limited to, a blood vessel (e.g., carotid artery, aorta, and the like) sample, a lung tissue sample, a SMC sample, a renal sample, and a sample from any tissue undergoing wound healing.

By “candidate anti-CTHRC1 drug,” as the term is used herein, is meant a compound that when contacted with a cell, reduces the level of expression of a nucleic acid encoding a CTHRC1 protein in the cell compared with the level of CTHRC1 expression in that cell prior to contacting the cell with the compound or which reduces the level of expression in the cell compared with the level of CTHRC1 expression in an otherwise identical cell which is not contacted with the compound.

A “cartilage disease,” is any disease, disorder or condition associated with, or mediated by, abnormal cartilage formation, modeling, and the like, compared to cartilage that is known to not be diseased. Typically, cartilage diseases are mediated by a mutation in a collagen gene resulting in misfolding of the resultant mutant collagen protein compared to the normal protein, thereby resulting in impaired function.

As used herein, the term “CTHRC1” means any adventitia-induced and bone expressed (AIBE) molecule (previously referred to as REMODEL and/or REMODELIN) now termed “collagen triple helix repeat containing 1” (CTHRC1), having significant sequence identity with CTHRC1 disclosed herein. More specifically, the putative CTHRC1 shares at least about 33% sequence identity with at least one of a nucleic acid having the sequence SEQ ID NO:1 and a nucleic acid having the sequence SEQ ID NO:3. More preferably, the nucleic acid encoding CTHRC1 has at least about 35% identity, even more preferably, at least about 40% identity, yet more preferably, at least about 45% identity, even more preferably, at least about 50% identity, more preferably, at least about 55% identity, even more preferably, at least about 60% identity, yet more preferably, at least about 65% identity, more preferably, at least about 70% identity, yet more preferably, at least about 75% identity, even more preferably, at least about 80% identity, more preferably, at least about 85% identity, yet more preferably, about 90% identity, even more preferably, at least about 95% identity, and most preferably, at least about 99% sequence identity with at least one of SEQ ID NO:1 and SEQ ID NO:3 disclosed herein. Even more preferably, the nucleic acid is at least one of SEQ ID NO:1 and SEQ ID NO:3. Further, the biological activity of a CTHRC1 preferably includes inhibition of expression of the nucleic acid encoding the CTHRC1 protein by a soluble TGF-β receptor type II (TGF-βRII), which blocks TGF-β signaling. Further, preferably, the biological activity of a CTHRC1 molecule includes induction of expression of the nucleic acid by TGF, induction of expression of the nucleic acid encoding a CTHRC1 in a blood vessel following vessel injury, induction of expression of the nucleic acid encoding the protein in fibroblasts during wound healing, expression in osteoblasts during bone formation, involvement in cell-cell and cell-matrix interaction, detectable decreased collagen matrix production, and affecting cell viability such as by, for example, affecting the life span of a cell.

Further, the data disclosed elsewhere herein demonstrate that CTHRC1 plays an important role in bone growth. In one embodiment, transgenic mice over-expressing CTHRC1, similar to data obtained using frog embryos which exhibited failure of dorsal closure, exhibited spina bifida-like effects. Therefore, the term “CTHRC1” encompasses a nucleic acid that, when over-expressed in a mammalian embryo, mediates or is associated with altered bone growth, bone density, and/or spina bifida-like phenotype.

Unless otherwise indicated, “CTHRC1” encompasses all known CTHRC1s (e.g., rat CTHRC1_{S}, rat CTHRC1_{L}, and human CTHRC1), and CTHRC1s to be discovered, including but not limited to, mouse CTHRC1, having the characteristics and/or physical features of the CTHRC1 disclosed herein.

However, the present invention does not include the isolated nucleic acids having the sequences designated by the following GenBank Accession Numbers: AA335862 (sharing about 87% identity with CTHRC1 over about 373 nucleotides); C01758 (sharing about 87% identity with CTHRC1 over about 356 nucleotides); AA335551 (sharing about 87% identity with CTHRC1 over about 334 nucleotides); and AA406425 (sharing about 88% identity with CTHRC1 over about 312 nucleotides); R46857; AA584310; D79314; A1085616; D62262; AA482398; AA482544; A1359844; A1352209; A1239604; A1218433; AI081084; A1074870; A1074769; AA974239; AA969841; AA857920; AA723450; AA410434; AA738416; A1370649; AA507081.

“CTHRC1 expression-inhibiting amount,” as used herein, means any amount of a substance or molecule that detectably decreases the level of CTHRC1 expression, amount, and/or activity compared with the level of CTHRC1 expression, amount, and/or activity in the absence of the substance or molecule. Thus, any amount that mediates a detectable decrease in: the amount of CTHRC1 present, the level of CTHRC1 mRNA expression, and/or the ability of CTHRC1 to form necessary ligand/receptor interactions, is encompassed in the present invention. The assays by which these conditions are examined are well-known in the art and several are exemplified herein.

“CTHRC1 expression-increasing amount,” as used herein, means any amount of a substance or molecule that detectably increases the level of CTHRC1 expression, amount, and/or activity compared with the level of CTHRC1 expression, amount, and/or activity in the absence of the substance or molecule. Thus, any amount that mediates a detectable increase in: the amount of CTHRC1 present, the level of CTHRC1 mRNA expression, and/or the ability of CTHRC1 to form necessary ligand/receptor interactions, is encompassed in the present invention. The assays by which these conditions are examined are well-known in the art and several are exemplified herein.

As used herein, an “effective amount” of CTHRC1 means any amount of CHTRC1 that mediates a detectable decrease in the level of collagen matrix production by a cell, tissue, organ, and the like, compared with the level of collagen matrix production by the same cell, tissue, organ, prior to administration of CTHRC1, or by an otherwise identical cell, tissue or organ to which CTHRC1 is not administered. Methods for assessing the level of collagen matrix production by a cell, tissue or organ are well-known in the art, including those described elsewhere herein, and also encompass such methods as are developed in the future. Moreover, administration of CTHRC1 encompasses, and is not limited to, administration of CTHRC1, or a fragment thereof, as well as administration of a nucleic acid encoding the same. Methods for administering a protein of interest are well-known in the art and are therefore not discussed further herein.

By the term “CTHRC1-like activity,” as used herein, refers to the ability of a molecule or compound to be induced by TGF-β, selectively induced in adventitia of injured vessels, to cause phenotypic abnormalities in amphibian embryos such as those disclosed herein (e.g., split tail, abnormal head development, lack of mesoderm development upon FGF-induction, failure of dorsal closure, and the like), to exhibit detectably increased expression in injured vessel adventitia compared with uninjured vessels or with the neointima of injured or uninjured vessels, the ability to induce adventitial cell proliferation, to be inhibited by a soluble TGF-β receptor II (which inhibits TGF-β signaling), the ability to be induced in fibroblasts during wound healing, the ability to be expressed by osteoblasts during bone formation, the ability to be expressed in osteoblasts adjacent to mineralized bone, the ability to be strongly expressed along full thickness skin incisions undergoing wound healing and remodeling, the ability to mediate cell death in endothelial cells when overexpressed, the ability to inhibit cell adhesion and cell-cell interaction when an antisense nucleic complementary to the nucleic acid encoding the molecule is expressed in a cell, the ability to affect collagen matrix production, the ability to affect remodeling, restenosis, and fibrosis, and the ability to mediate excessive or insufficient wound healing responses, scarring, keloids, bone formation, bone density, lack of dorsal closure, spina bifida-like effects, fracture healing, and the like. Further, it includes affecting BMP (e.g., BMP1, BMP4, and the like) mRNA expression, the level of BMP in a cell, as well as affecting chordin level, osteopontin level, propeptide processing, and the like, in a cell. It also includes affecting collagen deposition, bone matrix formation, expression of collagen mRNA, and increasing and/or decreasing the level of collagen, procollagen, or both, in a cell.

“BMP1 inhibiting amount of CTHRC1,” as used herein, means any amount of CTHRC1, or an active portion thereof, that detectably decreases the level of BMP1 expression, amount, and/or activity compared with the level of BMP1 expression, amount, and/or activity in the absence of the CTHRC1. Thus, any amount that mediates a detectable decrease in: the amount of BMP1 present, the level of BMP1 mRNA expression, and/or the ability of BMP1 to process a propeptide (e.g., procollagen, pro-lysyl-oxidase, and the like), to affect collagen deposition, the amount of collagen mRNA expression, the amount of procollagen, the amount of collagen, the amount of chordin, among others, is encompassed in the present invention. The assays by which these conditions are examined are well-known in the art and several are exemplified herein.

By “complementary to a portion or all of the nucleic acid encoding CTHRC1” is meant a sequence of nucleic acid which does not encode a CTHRC1 protein. Rather, the sequence which is being expressed in the cells is identical to the non-coding strand of the nucleic acid encoding a CTHRC1 protein and thus, does not encode CTHRC1 protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

As used herein, the term “constrictive remodeling” (also known as inward remodeling or negative remodeling) means a physiologic or pathologic response of a blood vessel to a stimulus resulting in a reduction of vessel diameter and lumen diameter. Such a stimulus could be provided by, for example, but not limited to, a change in blood flow or an angioplasty procedure.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

“Fibrosis”, as the term is used herein, refers to formation of increased collagenous extracellular matrix, as compared with the otherwise normal extracellular matrix not associated with any disease state or condition.

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, preferably, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 650 nucleotides, yet even more preferably, at least about 650 to about 800, more preferably, from about 800 to about 1000 nucleotides, preferably, at least about 1000 to about 1100 nucleotides, even more preferably, at least about 1100 nucleotides to about 1200 nucleotides, yet even more preferably, at least about 1200 to about 1210, even more preferably, at least about 1210 nucleotides to about 1220 nucleotides, yet even more preferably, at least about 1220 to about 1225, and most preferably, the nucleic acid fragment will be greater than about 1230 nucleotides in length.

However, as applied to a nucleic acid, the term fragment as used herein does not encompass the following isolated nucleic acids as referred to by their GenBank Accession numbers: AA335862 (sharing about 87% identity over about 373 nucleotides with CTHRC1 cDNA); C01758 (sharing about 87% identity over about 356 nucleotides with CTHRC1 cDNA); AA335551 (sharing about 87% identity over about 334 nucleotides with CTHRC1 cDNA); AA406425 (sharing about 88% identity over about 312 nucleotides with CTHRC1 cDNA); R46857; AA5843 10; D79314; A1085616; D62262; AA482398; AA482544; A1359844; A1352209; A1239604; A1218433; A1081084; A1074870; A1074769; AA974239; AA969841; AA857920; AA723450; AA410434; AA738416; A1370649; AA507081.

As applied to a protein, a “fragment” of CTHRC1 is about 20 amino acids in length. More preferably, the fragment of a CTHRC1 is about 30 amino acids, even more preferably, at least about 40, yet more preferably, at least about 60, even more preferably, at least about 80, yet more preferably, at least about 100, even more preferably, about 100, and more preferably, at least about 150, more preferably, at least about 200, yet more preferably, at least about 240, even more preferably, at least about 243, yet more preferably, at least about 250, even more preferably, about 270, and more preferably, at least about 277 amino acids in length amino acids in length. A fragment of CTHRC1 further encompasses a cleavage fragment of CTHRC1 produced by a protease (e.g., collagenase, MMP-1, and the like), and encompasses, inter alia, both long and short cleavage fragments of CTHRC1 produced by MMP-1, as disclosed elsewhere herein. That is, a “fragment” of CTHRC1 encompasses, among many others, a longer cleavage fragment comprising about 170 amino acid residues (cCTHRC1_{L}; [SEQ ID NO:11]), as well as a shorter cleavage fragment comprising about 164 amino acid residues (cCTHRC1_{S}; [SEQ ID NO:13]) produced by a protease such as, but not limited to, collagenase, a matrix metalloprotease, and the like.

A “genomic DNA” is a DNA strand which has a nucleotide sequence homologous with a gene. By way of example, both a fragment of a chromosome and a cDNA derived by reverse transcription of a mammalian mRNA are genomic DNAs.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

In addition, when the terms “homology” or “identity” are used herein to refer to the nucleic acids and proteins, it should be construed to be applied to homology or identity at both the nucleic acid and the amino acid sequence levels.

A first oligonucleotide anneals with a second oligonucleotide with “high stringency” or “under high stringency conditions” if the two oligonucleotides anneal under conditions whereby only oligonucleotides which are at least about 60%, more preferably at least about 65%, even more preferably at least about 70%, yet more preferably at least about 80%, and preferably at least about 90% or, more preferably, at least about 95% complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, In: Molecular Cloning: A Laboratory Manual, Cold Spring Harbor Laboratory, New York; Sambrook and Russell, 2001, In: Molecular Cloning, A Laboratory Approach, Cold Spring Harbor Press, Cold Spring Harbor, N.Y.; and Ausubel et al., 2002, In: Current Protocols in Molecular Biology, John Wiley & Sons, NY).

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul et al. (1990, J. Mol. Biol. 215:403-410), and can be accessed, for example, at the National Center for Biotechnology Information (NCBI) world wide web site of the National Library of Medicine (NLM), at the National Institutes of Health (NIH). BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used using the programs that are available at the National Center for Biotechnology Information (NCBI) world wide web site of the National Library of Medicine (NLM), at the National Institutes of Health (NIH).

The percent identity between two sequences can be determined using techniques similar to those described above, with or without allowing gaps. In calculating percent identity, typically exact matches are counted.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the mouse proteins described herein, are within the scope of the invention. Nucleic acid molecules corresponding to natural allelic variants and homologs of a cDNA of the invention can be isolated based on their identity to mouse nucleic acid molecules using the mouse cDNAs, or a portion thereof, as a hybridization probe according to standard hybridization techniques under stringent hybridization conditions. For example, a homolog of a nucleic acid encoding a rat CTHRC1 protein of the invention can be isolated based on its hybridization with a nucleic acid molecule encoding all or part of rat and/or human CTHRC1 under high stringency conditions.

As used herein, an “instructional material” includes a publication, a recording, a diagram, or any other medium of expression which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Neointima formation,” as that term is used herein, means the thickening and enlargement of the tunica intima of a blood vessel due to accumulation of cells and extracellular matrix in this layer of the vessel.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

Preferably, when the nucleic acid encoding the desired protein further comprises a promoter/regulatory sequence, the promoter/regulatory is positioned at the 5′ end of the desired protein coding sequence such that it drives expression of the desired protein in a cell. Together, the nucleic acid encoding the desired protein and its promoter/regulatory sequence comprise a “transgene.”

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “polyadenylation sequence” is a polynucleotide sequence which directs the addition of a poly A tail onto a transcribed messenger RNA sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate α-nitrophenyl-β-galactoside to the medium (Gerhardt et al., eds., 1994, Methods for General and Molecular Bacteriology, American Society for Microbiology, Washington, D.C., p. 574).

A “restriction site” is a portion of a double-stranded nucleic acid which is recognized by a restriction endonuclease.

A portion of a double-stranded nucleic acid is “recognized” by a restriction endonuclease if the endonuclease is capable of cleaving both strands of the nucleic acid at the portion when the nucleic acid and the endonuclease are contacted.

By the term “specifically binds,” as used herein, is meant a compound, e.g., a protein, a nucleic acid, an antibody, and the like, which recognizes and binds a specific molecule, but does not substantially recognize or bind other molecules in a sample.

A first oligonucleotide anneals with a second oligonucleotide “with high stringency” if the two oligonucleotides anneal under conditions whereby only oligonucleotides which are at least about 73%, more preferably, at least about 75%, even more preferably, at least about 80%, even more preferably, at least about 85%, yet more preferably, at least about 90%, and most preferably, at least about 95%, complementarily anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, Molecular Cloning: A Laboratory Manual, Cold Spring Harbor Laboratory, New York).

As used herein, the term “transgene” means an exogenous nucleic acid sequence which exogenous nucleic acid is encoded by a transgenic cell or mammal.

A “recombinant cell” is a cell that comprises a transgene. Such a cell may be a eukaryotic cell or a prokaryotic cell. Also, the transgenic cell encompasses, but is not limited to, an embryonic stem cell comprising the transgene, a cell obtained from a chimeric mammal derived from a transgenic ES cell where the cell comprises the transgene, a cell obtained from a transgenic mammal, or fetal or placental tissue thereof, and a prokaryotic cell comprising the transgene.

By the term “exogenous nucleic acid” is meant that the nucleic acid has been introduced into a cell or an animal using technology which has been developed for the purpose of facilitating the introduction of a nucleic acid into a cell or an animal.

By “tagged” polypeptide is meant any protein of interest which, when linked by a peptide bond to a peptide comprising a detectable amino acid sequence, can be used to localize the protein of interest, as well as to purify it, to immobilize it, or to in any way isolate or enrich for the protein of interest for use in, inter alia, a binding assay, to produce an antibody thereto, or to otherwise study its biological properties and/or function.

“TGF-β signaling,” as the term is used herein is any signal mediated by TGF-β interaction with a cognate receptor, e.g., TGF-β receptor type II. One result of such interaction is an increase in CTHRC1 in a cell comprising the receptor on its surface. Other effects of TGF-β signaling are well-known in the art and can be assessed using standard methods also known in the relevant art.

“Soluble TGF-β receptor II” encompasses a molecule such as, but not limited to, a recombinant TGF-β receptor II fusion peptide comprising the extracellular domain of a type II TGF-β receptor (e.g., rabbit type II TGF-β receptor) fused to, for instance, the Fc portion of an antibody (e.g., human IgG1), which construct retains the ability to bind TGF-β ligands (Smith et al., 1999, Circ. Res. 84:1212-1222) thereby affecting the interaction of a ligand with the cell-associated receptor.

As used herein, the term “transgenic mammal” means a mammal, the germ cells of which comprise an exogenous nucleic acid.

As used herein, to “treat” means reducing the frequency with which symptoms of arterial restenosis, adventitial fibrosis, constrictive remodeling, associated with production of extracellular collagen matrix, excessive or insufficient wound healing responses, scarring, keloids, bone formation, fracture healing, and the like, are experienced by a patient.

By the term “vector” as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of the CTHRC1 protein or nucleic acid encoding a mammalian CTHRC1, to the patient, or the vector may be a non-viral vector which is suitable for the same purpose.

Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

A “knock-out targeting vector,” as the term is used herein, means a vector comprising two nucleic acid sequences each of which is complementary to a nucleic acid regions flanking a target sequence of interest which is to be deleted and/or replaced by another nucleic acid sequence. The two nucleic acid sequences therefore flank the target sequence which is to be removed by the process of homologous recombination

Description

I. Isolated Nucleic Acids

A. Sense Nucleic Acids

The present invention includes an isolated nucleic acid encoding a mammalian collagen triple helix repeat containing 1, CTHRC1, molecule (previously referred to as adventitia-inducible and bone expressed (AIBE) protein, REMODEL, and REMODELIN), or a fragment thereof, wherein the nucleic acid shares at least about 33% identity with at least one nucleic acid having the sequence of (SEQ ID NO:1) and (SEQ ID NO:3). Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:1 and SEQ ID NO:3 disclosed herein. Even more preferably, the nucleic acid is at least one of SEQ ID NO:1 and SEQ ID NO:3.

The present invention includes an isolated nucleic acid encoding rat CTHRC1, or a fragment thereof, wherein the nucleic acid shares at least about 33% homology with a nucleic acid having the sequence SEQ ID NO:1. Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the rat CTHRC1 disclosed herein (SEQ ID NO:1). Even more preferably, the nucleic acid is SEQ ID NO:1.

The present invention includes an isolated nucleic acid encoding human CTHRC1, or a fragment thereof, wherein the nucleic acid shares at least about 33% homology with human CTHRC1 (SEQ ID NO:3). Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the human CTHRC1 disclosed herein (SEQ ID NO:3). Even more preferably, the nucleic acid is SEQ ID NO:3.

In another aspect, the present invention includes an isolated nucleic acid encoding a mammalian CTHRC1, or a fragment thereof, wherein the protein encoded by the nucleic acid shares greater than about 6% homology with the amino acid sequence of at least one of SEQ ID NO:2 (rat CTHRC Is), SEQ ID NO:4 (human CTHRC1), and SEQ ID NO:5 (rat CTHRC1_{L}). That is, searching GenBank databases disclosed that CTHRC1 shares about 62% sequence identity with a portion of the sequence GenBank Acc. No. P27393, collagen alpha-2 (IV) chain precursor, over a stretch of about 35 amino acids. Full-length CTHRC1 protein comprises about 243 amino acids such that full-length CTHRC1 shares about 5.7% overall sequence identity with collagen alpha-2 (IV) chain precursor (i.e., GenBank Acc. No. P27393).

Preferably, the protein encoded by the isolated nucleic acid encoding CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:2, SEQ ID NO:4, and SEQ ID NO:5. Even more preferably, the CTHRC1 protein encoded by the nucleic acid is at least one of SEQ ID NO:2, SEQ ID NO:4, and SEQ ID NO:5.

In another aspect, the present invention includes an isolated nucleic acid encoding rat CTHRC1, or a fragment thereof, wherein the protein encoded by the nucleic acid shares at least about 6% homology with the amino acid sequence of SEQ ID NO:2. Preferably, the protein encoded by the isolated nucleic acid encoding CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to rat CTHRC1 disclosed herein (SEQ ID NO:2). Even more preferably, the rat CTHRC1 protein encoded by the nucleic acid is SEQ ID NO:2.

In another aspect, the present invention includes an isolated nucleic acid encoding human CTHRC1, or a fragment thereof, wherein the protein encoded by the nucleic acid shares at least about 6% homology with the amino acid sequence of SEQ ID NO:4. Preferably, the protein encoded by the isolated nucleic acid encoding CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the human CTHRC1 disclosed herein (SEQ ID NO:4). Even more preferably, the human CTHRC1 protein encoded by the nucleic acid is SEQ ID NO:4.

One skilled in the art would understand, based upon the disclosure provided herein, that a nucleic acid encoding a rat CTHRC1 (SEQ ID NO:1) can be alternatively translated to produce an alternate rat CTHRC1 protein comprising 245 amino acids (rat CTHRC1_{S}; SEQ ID NO:2) and a protein comprising an additional 32 amino acid residues at the N-terminus (i.e., the 277 amino acid long form of CTHRC1 designated CTHRC1_{L} [SEQ ID NO:5]) since the nucleic acid encoding rat CTHRC1 (SEQ ID NO:1) comprises two putative transcriptional start sites at positions 19 and 116 (FIG. 4A) that are compatible with the Kozak rule.

Therefore, in another aspect, the present invention includes an isolated nucleic acid encoding rat CTHRC1, or a fragment thereof, wherein the protein encoded by the nucleic acid shares at least about 6% homology with the amino acid sequence of SEQ ID NO:5 (i.e., 277 amino acid rat CTHRC1_{L}). Preferably, the protein encoded by the isolated nucleic acid encoding CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the rat CTHRC1_{L} disclosed herein (SEQ ID NO:5). Even more preferably, the rat CTHRC1_{L} protein encoded by the nucleic acid is SEQ ID NO:5.

In yet another aspect, the data disclosed herein demonstrate that CTHRC1 comprises at least two sites where the protein can be cleaved by, e.g., a matrix metalloprotease, such as, MMP-1, and by a protease, such as, but not limited to, collagenase. Indeed, the data presented herein demonstrate that CTHRC1 expressed in certain mammalian cells migrates in a gel according to its size as several bands, indicating the protein is cleaved in those cells. Accordingly, the skilled artisan would appreciate, based upon the disclosure provided herein, that the invention encompasses cleaved CTHRC1, wherein the molecule is cleaved by, among others, MMP-1 and collagenase.

Based on the predicted cleavage sites for MMP-1, as well as the data disclosed elsewhere herein, one skilled in the art would appreciate that the invention encompasses a nucleic acid encoding a cleaved CTHRC1 polypeptide, wherein the nucleic acid sequence shares at least about 33% identity with at least one nucleic acid having the sequence of (SEQ ID NO:10) and (SEQ ID NO:12). Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:10 (cleaved CTHRC1 longer fragment) and SEQ ID NO:12 (cleaved CTHRC1 shorter fragment) disclosed herein. Even more preferably, the nucleic acid is at least one of SEQ ID NO:10 and SEQ ID NO:12.

The present invention includes an isolated nucleic acid encoding human cleaved CTHRC1, longer fragment (cCTHRC1_{L}), or a fragment thereof, wherein the nucleic acid shares at least about 33% homology with a nucleic acid having the sequence SEQ ID NO:10. Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the cCTHRC1_{L} disclosed herein (SEQ ID NO:10). Even more preferably, the nucleic acid is SEQ ID NO:10.

The present invention includes an isolated nucleic acid encoding human cleaved CTHRC1, shorter fragment (cCTHRC1_{S}), or a fragment thereof, wherein the nucleic acid shares at least about 33% homology with human cleaved CTHRC1 (shorter fragment) (SEQ ID NO:12). Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to the human cCTHRC1_{S} disclosed herein (SEQ ID NO:12). Even more preferably, the nucleic acid is SEQ ID NO:12.

In another aspect, the present invention includes an isolated nucleic acid encoding a human cleaved CTHRC1, or a fragment thereof, wherein the protein encoded by the nucleic acid shares greater than about 33% homology with the amino acid sequence of at least one of SEQ ID NO:11 (human cleaved CTHRC1 longer fragment; cCTHRC1_{L}), and SEQ ID NO:13 (human cleaved CTHRC1, shorter fragment; cCTHRC1_{S}). Preferably, the protein encoded by the isolated nucleic acid encoding cCTHRC1_{L} and cCTHRC1_{S} is at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:11, and SEQ ID NO:13. Even more preferably, the cleaved CTHRC1 protein encoded by the nucleic acid is at least one of SEQ ID NO:11, and SEQ ID NO:13.

In another aspect, the present invention includes an isolated nucleic acid encoding human cleaved CTHRC1, longer fragment (cCTHRC1_{L}), or a fragment thereof, wherein the protein encoded by the nucleic acid shares at least about 33% homology with the amino acid sequence of SEQ ID NO:11. Preferably, the protein encoded by the isolated nucleic acid encoding cCTHRC1_{L} is at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to cCTHRC1_{L} disclosed herein (SEQ ID NO:11). Even more preferably, the cCTHRC1_{L} protein encoded by the nucleic acid is SEQ ID NO:11.

In yet another aspect, the present invention includes an isolated nucleic acid encoding human cleaved CTHRC1, shorter fragment (cCTHRC1_{S}), or a fragment thereof, wherein the protein encoded by the nucleic acid shares at least about 33% homology with the amino acid sequence of SEQ ID NO:13. Preferably, the protein encoded by the isolated nucleic acid encoding cCTHRC1_{S} is at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to cCTHRC1_{S} disclosed herein (SEQ ID NO:13). Even more preferably, the cCTHRC1_{S} protein encoded by the nucleic acid is SEQ ID NO:13.

One skilled in the art would appreciate, based upon the disclosure provided herein, that a mouse CTHRC1 homolog likely exists and can be readily identified and isolated using the methods described herein using the sequence data disclosed herein regarding the highly-conserved rat and mouse homologs. Thus, the present invention encompasses additional CTHRC1s that can be readily identified based upon the disclosure provided herein, including, but not limited to, mouse CTHRC1.

The isolated nucleic acid of the invention should be construed to include an RNA or a DNA sequence encoding a CTHRC1 protein of the invention (excluding the isolated nucleic acids referred to by the following GenBank Accession Nos: AA335862; C01758; AA335551; AA406425; R46857; AA584310; D79314; AI085616; D62262; AA482398; AA482544; A1359844; A1352209; A1239604; A1218433; A1081084; A1074870; A1074769; AA974239; AA969841; AA857920; AA723450; AA410434; AA738416; A1370649; AA507081), and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding CTHRC1 proteins can such as those present in other species of mammals (e.g., ape, gibbon, bovine, ovine, equine, porcine, canine, feline, and the like) be obtained by following the procedures described herein in the experimental details section for the isolation of the rat, and human CTHRC1 nucleic acids encoding CTHRC1 polypeptides as disclosed herein (e.g., screening of genomic or cDNA libraries), and procedures that are well-known in the art (e.g., reverse transcription PCR using mRNA samples) or to be developed.

Further, any number of procedures may be used for the generation of mutant, derivative or variant forms of CTHRC1 using recombinant DNA methodology well known in the art such as, for example, that described in Sambrook et al. (1989, Molecular Cloning: A Laboratory Manual, Cold Spring Harbor Laboratory Press, New York), Ausubel et al. (1997, Current Protocols in Molecular Biology, Green & Wiley, New York), Sambrook and Russell (2001, Molecular Cloning, A Laboratory Approach, Cold Spring Harbor Press, Cold Spring Harbor, N.Y.), and Ausubel et al. (2002, Current Protocols in Molecular Biology, John Wiley & Sons, NY).

Procedures for the introduction of amino acid changes in a protein or polypeptide by altering the DNA sequence encoding the polypeptide are well known in the art and are also described in Sambrook et al. (1989, supra); Ausubel et al. (1997, supra); Sambrook and Russell (2001, supra); and Ausubel et al. (2002, supra), among many other treatises.

Similarly, procedures are well-known for deletion of a portion of a nucleic acid encoding a protein of interest. That is, where the various domains of a protein are known, such as disclosed herein for CTHRC1 (see, e.g., FIG. 3 and FIG. 28), the skilled artisan, based upon the disclosure provided herein regarding, inter alia, the importance and/or role of various domains of the peptide, would appreciate that the invention comprises various deletion and/or site-specific mutants comprising mutations at desired domains of CTHRC1. One such mutant comprises a deletion of the nucleic acid encoding the collagen domain (at about amino acid 60 to 95) of CTHRC1 (FIG. 28), as well as deletion mutants wherein the nucleic acid encoding the N-terminus amino acid residues of the protein is deleted in accordance with the potential MMP-1 and/or collagenase cleavage sites as disclosed elsewhere herein, to produce a polypeptide comprising an amino acid sequence such as the amino acid sequence of SEQ ID NO:11 and the sequence of SEQ ID NO:13.

The invention includes a nucleic acid encoding a mammalian CTHRC1 wherein a nucleic acid encoding a tag polypeptide is covalently linked thereto. That is, the invention encompasses a chimeric nucleic acid wherein the nucleic acid sequences encoding a tag polypeptide is covalently linked to the nucleic acid encoding at least one of rat CTHRC1 and human CTHRC1. Such tag polypeptides are well known in the art and include, for instance, green fluorescent protein (GFP), an influenza virus hemagglutinin tag polypeptide, myc, myc-pyruvate kinase (myc-PK), His₆, maltose biding protein (MBP), a FLAG tag polypeptide, and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize CTHRC1 within a cell, a tissue (e.g., a blood vessel, bone, and the like), and/or a whole organism (e.g., an amphibian and/or a mammalian embryo, and the like), detect CTHRC1 if secreted from a cell, and to study the role(s) of CTHRC1 in a cell. Further, addition of a tag polypeptide facilitates isolation and purification of the “tagged” protein such that the proteins of the invention can be produced and purified readily. Thus, the present invention encompasses a CTHRC1 polypeptide, or a portion thereof, further comprising a “tag” polypeptide.

B. Antisense Nucleic Acids

In certain situations, it may be desirable to inhibit expression of CTHRC1 and the invention therefore includes compositions useful for inhibition of CTHRC1 expression. Thus, the invention features an isolated nucleic acid complementary to a portion or all of a nucleic acid encoding a mammalian CTHRC1, which nucleic acid is in an antisense orientation with respect to transcription. Preferably, the antisense nucleic acid is complementary with a nucleic acid having at least about 33% homology with at least one of SEQ ID NO:1, SEQ ID NO:3, SEQ ID NO:10, and SEQ ID NO:12, or a fragment thereof. Preferably, the nucleic acid is at least about 35% homologous, more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to a nucleic acid complementary to a portion or all of a nucleic acid encoding a mammalian CTHRC1 having the sequence of at least one of SEQ ID NO:1, SEQ ID NO:3, SEQ ID NO:10, and SEQ ID NO:12, or a fragment thereof, which is in an antisense orientation with respect to transcription. Most preferably, the nucleic acid is complementary to a portion or all of a nucleic acid that is at least one of SEQ ID NO:1, SEQ ID NO:3, SEQ ID NO:10, and SEQ ID NO:12, or a fragment thereof. Such antisense nucleic acid serves to inhibit the expression, function, or both, of an adventitia-inducible and bone expressed, collagen triple helix repeat containing 1 (CTHRC1) molecule, whether full-length or as a cleavage fragment produced by a protease (e.g., MMP-1, collagenase, and the like).

In one aspect, the invention includes an isolated nucleic acid complementary to a portion or all of a nucleic acid encoding a mammalian CTHRC1 molecule, which nucleic acid is in an antisense orientation with respect to transcription. Preferably, the antisense nucleic acid is complementary with a nucleic acid having at least about 33% homology with SEQ ID NO:1, or a fragment thereof. Preferably, the antisense nucleic acid is complementary with a nucleic acid having at least about 35% identity, more preferably, at least about 40% identity, even more preferably, at least about 45% identity, yet more preferably, at least about 50% identity, more preferably, at least about 55% identity, more preferably, at least about 60% identity, even more preferably, at least about 65% identity, yet more preferably, at least about 70% identity, more preferably, at least about 75% identity, even more preferably, at least about 80% identity, yet more preferably, at least about 85% identity, more preferably, at least about 90% identity, even more preferably, at least about 95% identity, and most preferably, at least about 99% identity with a nucleic acid complementary to a portion or all of a nucleic acid encoding a mammalian CTHRC1 having the sequence SEQ ID NO:1

Most preferably, the nucleic acid is complementary to a portion or all of a nucleic acid that is SEQ ID NO:1, or a fragment thereof. Such antisense nucleic acid serves to inhibit the expression, function, or both, of an adventitia-inducible and bone expressed CTHRC1 molecule.

In another aspect, the invention includes an isolated nucleic acid complementary to a portion or all of a nucleic acid encoding a mammalian CTHRC1 molecule, which nucleic acid is in an antisense orientation with respect to transcription. Preferably, the antisense nucleic acid is complementary with a nucleic acid having at least about 35% identity, more preferably, at least about 40% identity, even more preferably, at least about 45% identity, yet more preferably, at least about 50% identity, more preferably, at least about 55% identity, more preferably, at least about 60% identity, even more preferably, at least about 65% identity, yet more preferably, at least about 70% identity, more preferably, at least about 75% identity, even more preferably, at least about 80% identity, yet more preferably, at least about 85% identity, more preferably, at least about 90% identity, even more preferably, at least about 95% identity, and most preferably, at least about 99% identity with a nucleic acid complementary to a nucleic acid encoding a mammalian CTHRC1 having the sequence SEQ ID NO:3, or a fragment thereof. Most preferably, the nucleic acid is complementary to a portion or all of a nucleic acid that is SEQ ID NO:3, or a fragment thereof. Such antisense nucleic acid serves to inhibit the expression, function, or both, of an adventitia-inducible and bone expressed CTHRC1 molecule.

Further, antisense nucleic acids complementary to all or a portion of a nucleic acid encoding CTHRC1 can be used to detect the expression of CTHRC1 mRNA in a cell, tissue, and/or organism, using, for example but not limited to, in situ hybridization. Thus, one skilled in the art would understand, based upon the disclosure provided herein, that the invention encompasses antisense nucleic acids that can be used as probes to assess CTHRC1 expression. Such antisense nucleic acids encompass, but are not limited to, a nucleic acid having the sequence SEQ ID NO:6.

In another aspect, the invention includes an isolated nucleic acid complementary to a portion or all of a nucleic acid encoding a mammalian cCTHRC1_{L} molecule, which nucleic acid is in an antisense orientation with respect to transcription. Preferably, the antisense nucleic acid is complementary with a nucleic acid having at least about 33% homology with SEQ ID NO:10, or a fragment thereof. Preferably, the antisense nucleic acid is complementary with a nucleic acid having at least about 35% identity, more preferably, at least about 40% identity, even more preferably, at least about 45% identity, yet more preferably, at least about 50% identity, more preferably, at least about 55% identity, more preferably, at least about 60% identity, even more preferably, at least about 65% identity, yet more preferably, at least about 70% identity, more preferably, at least about 75% identity, even more preferably, at least about 80% identity, yet more preferably, at least about 85% identity, more preferably, at least about 90% identity, even more preferably, at least about 95% identity, and most preferably, at least about 99% identity with a nucleic acid complementary to a portion or all of a nucleic acid encoding a mammalian cCTHRC1_{L} having the sequence SEQ ID NO:10.

Most preferably, the nucleic acid is complementary to a portion or all of a nucleic acid that is SEQ ID NO:10, or a fragment thereof. Such antisense nucleic acid serves to inhibit the expression, function, or both, of an adventitia-inducible and bone expressed cCTHRC1_{L} molecule.

In one aspect, the invention includes an isolated nucleic acid complementary to a portion or all of a nucleic acid encoding a mammalian cleaved CTHRC1, shorter fragment (cCTHRC1_{S}) molecule (SEQ ID NO:12), which nucleic acid is in an antisense orientation with respect to transcription. Preferably, the antisense nucleic acid is complementary with a nucleic acid having at least about 33% homology with SEQ ID NO:12, or a fragment thereof. Preferably, the antisense nucleic acid is complementary with a nucleic acid having at least about 35% identity, more preferably, at least about 40% identity, even more preferably, at least about 45% identity, yet more preferably, at least about 50% identity, more preferably, at least about 55% identity, more preferably, at least about 60% identity, even more preferably, at least about 65% identity, yet more preferably, at least about 70% identity, more preferably, at least about 75% identity, even more preferably, at least about 80% identity, yet more preferably, at least about 85% identity, more preferably, at least about 90% identity, even more preferably, at least about 95% identity, and most preferably, at least about 99% identity with a nucleic acid complementary to a portion or all of a nucleic acid encoding a mammalian cCTHRC1_{S} having the sequence SEQ ID NO:12.

Most preferably, the nucleic acid is complementary to a portion or all of a nucleic acid that is SEQ ID NO:12, or a fragment thereof. Such antisense nucleic acid serves to inhibit the expression, function, or both, of an adventitia-inducible and bone expressed cCTHRC1_{S} molecule.

Antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

II. Isolated Polypeptides

The invention also includes an isolated polypeptide comprising a mammalian CTHRC1. Preferably, the isolated polypeptide comprising a mammalian CTHRC1 is at least about 6% homologous to a polypeptide having the amino acid sequence of at least one of SEQ ID NO:2, SEQ ID NO:4, SEQ ID NO:5, SEQ ID NO:11, and SEQ ID NO:13. Preferably, the isolated polypeptide is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:2, SEQ ID NO:4, SEQ ID NO:5, SEQ ID NO:11, and SEQ ID NO:13. More preferably, the isolated polypeptide comprising a mammalian CTHRC1 is at least one of rat CTHRC1_{S}, human CTHRC1, rat CTHRC1L, and cleaved CTHRC1, both long (c CTHRC1_{L}) and short (CCTHRC1_{S}) cleavage forms thereof. Most preferably, the isolated polypeptide comprising a mammalian CTHRC1 is at least one of SEQ ID NO:2, SEQ ID NO:4, SEQ ID NO:5, SEQ ID NO:11, and SEQ ID NO:13.

The invention also includes an isolated polypeptide comprising a mammalian CTHRC1 molecule. Preferably, the isolated polypeptide comprising a mammalian CTHRC1 is at least about 6% homologous to a polypeptide having the amino acid sequence of SEQ ID NO:2. More preferably, the isolated polypeptide comprising a mammalian CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to rat CTHRC1_{S} (SEQ ID NO:2) More preferably, the isolated polypeptide comprising a mammalian CTHRC1 is rat CTHRC1_{S}. Most preferably, the isolated polypeptide comprising a mammalian CTHRC1 molecule is SEQ ID NO:2.

The invention also includes an isolated polypeptide comprising a mammalian CTHRC1 molecule. Preferably, the isolated polypeptide comprising a mammalian CTHRC1 molecule is at least about 6% homologous to a polypeptide having the amino acid sequence of SEQ ID NO:4. More preferably, the isolated polypeptide comprising a mammalian CTHRC1 is at least is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to SEQ ID NO:4. More preferably, the isolated polypeptide comprising a mammalian CTHRC1 molecule is human CTHRC1. Most preferably, the isolated polypeptide comprising a mammalian CTHRC1 molecule is SEQ ID NO:4.

The invention also includes an isolated polypeptide comprising a mammalian CTHRC1 molecule. Preferably, the isolated polypeptide comprising a mammalian CTHRC1 molecule is at least about 6% homologous to a polypeptide having the amino acid sequence of SEQ ID NO:5. More preferably, the isolated polypeptide comprising a mammalian CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to rat CTHRC1_{L}. More preferably, the isolated polypeptide comprising a mammalian CTHRC1 molecule is rat CTHRC1_{L}. Most preferably, the isolated polypeptide comprising a mammalian CTHRC1 molecule is SEQ ID NO:5.

The invention also includes an isolated polypeptide comprising a human cleaved CTHRC1, longer fragment (cCTHRC1_{L}) molecule. Preferably, the isolated polypeptide comprising a human cleaved CTHRC1, longer fragment, molecule is at least about 6% homologous to a polypeptide having the amino acid sequence of SEQ ID NO:11. More preferably, the isolated polypeptide comprising a mammalian CTHRC1 is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to cCTHRC1_{L} (SEQ ID NO:11). More preferably, the isolated polypeptide comprising a cleaved CTHRC₁ molecule is cCTHRC1_{L}. Most preferably, the isolated polypeptide comprising a cleaved CTHRC1 molecule is SEQ ID NO:11.

The invention also includes an isolated polypeptide comprising a human cleaved CTHRC1, shorter fragment, molecule (cCTHRC1_{S}). Preferably, the isolated polypeptide comprising a cCTHRC1_{S} molecule is at least about 6% homologous to a polypeptide having the amino acid sequence of SEQ ID NO:13. More preferably, the isolated polypeptide comprising a cCTHRC1_{S} is at least about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to cCTHRC1_{S} (SEQ ID NO:13). Most preferably, the isolated polypeptide comprising a cCTHRC1_{S} molecule is SEQ ID NO:13.

The present invention also provides for analogs of proteins or peptides which comprise a CTHRC1 as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “mutants,” “derivatives,” and “variants” of the peptides of the invention (or of the DNA encoding the same) which mutants, derivatives and variants are CTHRC1 peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the CTHRC1 peptide of the present invention.

A “biological property of a CTHRC1 protein” should be construed but not be limited to include, the ability of the expression of the peptide to be induced by TGF-β, the ability of the peptide to be expressed selectively in adventitia, the ability of the peptide to be induced by balloon-injury, the ability of the peptide to be expressed in bone, the ability of the peptide to be expressed in a mouse embryo commencing at about day 11.5 post coitus, the ability of a molecule to be selectively induced in adventitia of injured vessels, to cause phenotypic abnormalities in amphibian embryos such as those disclosed herein (e.g., split tail, abnormal head development, lack of mesoderm development upon FGF-induction, failure of dorsal closure, and the like), to exhibit increased expression only in injured vessel adventitia but not in uninjured vessels nor in the neointima of injured or uninjured vessels, the ability to induce adventitial cell proliferation, to be inhibited by a soluble TGF-β receptor II (which blocks TGF-β signaling), the ability to be induced in fibroblasts during wound healing, the ability to be expressed by osteoblasts during bone formation, the ability to mediate cell death in endothelial cells when overexpressed, the ability to affect cell adhesion and cell-cell interaction, the ability to affect bone density and/or bone growth, the ability to modulate collagen matrix production, fibrosis, restenosis, and the ability to mediate excessive or insufficient wound healing responses, scarring, keloids, bone formation, fracture healing, and the like.

Further, the invention should be construed to include naturally occurring variants or recombinantly derived mutants of CTHRC1 sequences, which variants or mutants render the protein encoded thereby either more, less, or just as biologically active as the full-length clones of the invention.

The nucleic acids, and peptides encoded thereby, are useful tools for elucidating the function(s) of CTHRC1 molecule in a cell. Further, nucleic and amino acids comprising mammalian CTHRC1 molecule are useful diagnostics which can be used, for example, to identify a compound that affects CTHRC1 expression and/or TGF-β signaling, and the like, and is a potential therapeutic drug candidate for arterial restenosis, to promote or inhibit wound healing, to inhibit scar tissue or keloid formation, to promote bone fracture healing, and the like. The nucleic acids, the proteins encoded thereby, or both, can be administered to a mammal to increase or decrease expression of CTHRC1 in the mammal. This can be beneficial for the mammal in situations where under or over-expression of CTHRC1 in the mammal mediates a disease or condition associated with altered expression of CTHRC1 compared with normal expression of CTHRC1 in a healthy mammal. Such conditions that can be affected by modulating CTHRC1 expression thereby providing a therapeutic benefit include, but are not limited to, wound healing, arterial injury, ossification, and the like. This is because, as more fully disclosed elsewhere herein, CTHRC1 is transiently expressed in (myo)fibroblasts in conditions associated with healing and repair following tissue injury. For instance, CTHRC1 is expressed in osteoblasts bone, which is undergoing constant remodeling. Additionally, over-expression of CTHRC1 during embryogenesis affects dorsal closure, bone density, and bone growth, and mediates and/or is associated with spina bifida-like effects all of which demonstrate the important biological role(s) of CTHRC1. Further, the data disclosed herein demonstrate that decreased level of CTHRC1 can provide a therapeutic benefit where collagen matrix production is desirable, such as, but not limited to, for increased bone healing, increased bone density, stabilizing plaque, and the like. Alternatively, the data disclosed herein demonstrate that an increased level of CTHRC1 can mediate decreased fibrosis, decreased restenosis, such that administration of CTHRC1 can thereby provide a substantial benefit under conditions where decreased collagen matrix formation is desired. The skilled artisan, based upon the disclosure provided herein, would readily appreciate under what condition, and in which disease or disorder of interest, it is desirable to increase or decrease the level of CTHRC1 in a cell, tissue, or organ.

Additionally, the nucleic and amino acids of the invention can be used to produce recombinant cells and transgenic non-human mammals which are useful tools for the study of CTHRC1 action, the identification of novel diagnostics and therapeutics for treatment, and for elucidating the cellular role(s) of CTHRC1, among other things. For instance, transgenic animals can be used to study bone related, wound healing related, and vascular disease related conditions, as extensively exemplified elsewhere herein.

Further, the nucleic and amino acids of the invention can be used diagnostically, either by assessing the level of gene expression or protein expression, to assess severity and prognosis of negative remodeling, arterial restenosis, vessel injury, fibrosis (renal and otherwise), bone growth, and the like. The nucleic acids and proteins of the invention are also useful in the development of assays to assess the efficacy of a treatment for preventing arterial restenosis, fibrosis, affecting bone density and bone growth, and the like. That is, the nucleic acids and polypeptides of the invention can be used to detect the effect of various therapies on CTHRC1 expression, thereby ascertaining the effectiveness of the therapies such as, but not limited to, assessment of treatment efficacies for restenosis, anti-fibrotic therapy in any tissue, therapies to promote wound healing in any tissue and therapies for bone formation including bone fracture healing.

III. Vectors

In other related aspects, the invention includes an isolated nucleic acid encoding a mammalian CTHRC1 operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, supra), and Ausubel et al. (1997, supra).

Expression of CTHRC1, either alone or fused to a detectable tag polypeptide, in cells which either do not normally express the CTHRC1 or which do not express CTHRC1 fused with a tag polypeptide, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding CTHRC1 may be accomplished by placing the nucleic acid encoding CTHRC1, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing CTHRC1 using a vector allows the isolation of large amounts of recombinantly produced protein. Further, where the lack or decreased level of CTHRC1 expression causes a disease, disorder, or condition associated with such expression, the expression of CTHRC1 driven by a promoter/regulatory sequence can provide useful therapeutics including, but not limited to, gene therapy whereby CTHRC1 is provided. A disease, disorder or condition associated with a decreased level of expression, level of protein, or decreased activity of the protein, for which administration of CTHRC1 can be useful can include, but is not limited to, bone formation, bone fracture healing, wound healing and repair in any tissue, and the like. Therefore, the invention includes not only methods of inhibiting CTHRC1 expression, translation, and/or activity, but it also includes methods relating to increasing CTHRC1 expression, protein level, and/or activity since both decreasing and increasing CTHRC1 expression and/or activity can be useful in providing effective therapeutics.

One skilled in the art would appreciate, based upon the disclosure provided herein, that because of the selective expression of CTHRC1 during wound healing in response to injury in any tissue, the promoter for CTHRC1 can be an excellent choice for targeting nucleic acid expression of a desired gene to a site of tissue injury.

Selection of any particular plasmid vector or other DNA vector is not a limiting factor in this invention and a wide plethora vectors is well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook, supra, and Ausubel, supra.

The invention thus includes a vector comprising an isolated nucleic acid encoding a mammalian CTHRC1. The incorporation of a desired nucleic acid into a vector and the choice of vectors is well-known in the art as described in, for example, Sambrook et al., supra, and Ausubel et al., supra.

The invention also includes cells, viruses, proviruses, and the like, containing such vectors. Methods for producing cells comprising vectors and/or exogenous nucleic acids are well-known in the art. See, e.g., Sambrook et al., supra; Ausubel et al., supra.

The nucleic acids encoding CTHRC1 can be cloned into various plasmid vectors. However, the present invention should not be construed to be limited to plasmids or to any particular vector. Instead, the present invention should be construed to encompass a wide plethora of vectors which are readily available and/or well-known in the art and no vector at all.

IV. Antisense Molecules and Ribozymes

Further, the invention includes a recombinant cell comprising an antisense nucleic acid which cell is a useful model for elucidating the role(s) of CTHRC1 in cellular processes. That is, the increased expression of CTHRC1 in balloon-injured vessels and, more specifically, in the adventitia thereof, indicate that CTHRC1 is involved in cell proliferation associated with constrictive remodeling and arterial restenosis. Accordingly, a transgenic cell comprising an antisense nucleic acid complementary to CTHRC1 but in an antisense orientation is a useful tool for the study of the mechanism(s) of action of CTHRC1 and its role(s) in the cell and for the identification of therapeutics that ameliorate the effect(s) of CTHRC1 expression.

One skilled in the art can appreciate, based upon the disclosure provided herein, that an antisense nucleic acid complementary to a nucleic acid encoding CTHRC1 can be used to transfect a cell and the cell can be studied to determine the effect(s) of altered expression of CTHRC1 in order to study the function(s) of CTHRC1 and to identity useful therapeutics and diagnostics.

Further, methods of decreasing CTHRC1 expression and/or activity in a cell can provide useful diagnostics and/or therapeutics for diseases, disorders or conditions mediated by or associated with increased CTHRC1 expression, increased level of CTHRC1 protein in a cell or secretion therefrom, and/or increased CTHRC1 activity. Such diseases, disorders or conditions include, but are not limited to, any condition associated with fibrosis, e.g., proliferation of fibroblasts with or without excessive fibrous tissue formation, and any condition associated with excessive bone formation or ectopic ossification (malignant or benign), and the like.

One skilled in the art will appreciate that one way to decrease the levels of CTHRC1 mRNA and/or protein in a cell is to inhibit expression of the nucleic acid encoding the protein. Expression of CTHRC1 may be inhibited using, for example, antisense molecules, and also by using ribozymes or double-stranded RNA as described in, for example, Wianny and Kernicka-Goetz (2000, Nature Cell Biol. 2:70-75).

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue (1993, U.S. Pat. No. 5,190,931).

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053, incorporated by reference herein in its entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of CTHRC1 can be designed by incorporating target sequences into the basic ribozyme structure which are complementary to the mRNA sequence of the CTHRC1 encoded by CTHRC1 or having at least about 33% homology to at least one of SEQ ID NO:1 and SEQ ID NO:3. Preferably, the sequence is at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of SEQ ID NO:1 and SEQ ID NO:3. Ribozymes targeting CTHRC1 may be synthesized using commercially available reagents (Applied Biosystems, Inc., Foster City, Calif.) or they may be genetically expressed from DNA encoding them.

More recently, a number of strategies relating to short interfering RNAs for inhibition of expression of a nucleic acid of interest have been described, and such techniques, as well as those to be developed in the future for inhibiting nucleic acid expression, are encompassed herein as would be appreciated by the skilled artisan armed with the teachings provided herein.

V. Recombinant Cells and Transgenic Non-Human Mammals

The invention includes a recombinant cell comprising, inter alia, an isolated nucleic acid encoding CTHRC1, an antisense nucleic acid complementary thereto, a nucleic acid encoding an antibody that specifically binds CTHRC1, and the like. In one aspect, the recombinant cell can be transiently transfected with a vector (e.g., a plasmid, and the like) encoding a portion of the nucleic acid encoding CTHRC1. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, fibroblasts, mouse stem cells, amphibian oocytes, osteoblasts, smooth muscle cells, endothelial cells, and the like.

In one aspect, the recombinant cell comprising an isolated nucleic acid encoding mammalian CTHRC1 is used to produce a transgenic non-human mammal. That is, the exogenous nucleic acid, or “transgene” as it is also referred to herein, of the invention is introduced into a cell, and the cell is then used to generate the non-human transgenic mammal. The cell into which the transgene is introduced is preferably an embryonic stem (ES) cell. However, the invention should not be construed to be limited solely to ES cells comprising the transgene of the invention nor to cells used to produce transgenic animals. Rather, a transgenic cell of the invention includes, but is not limited to, any cell derived from a transgenic animal comprising a transgene, a cell comprising the transgene derived from a chimeric animal derived from the transgenic ES cell, and any other comprising the transgene which may or may not be used to generate a non-human transgenic mammal.

Further, it is important to note that the purpose of transgene-comprising, i.e., recombinant, cells should not be construed to be limited to the generation of transgenic mammals. Rather, the invention should be construed to include any cell type into which a nucleic acid encoding a mammalian CTHRC1 is introduced, including, without limitation, a prokaryotic cell and a eukaryotic cell comprising an isolated nucleic acid encoding mammalian CTHRC1.

When the cell is a eukaryotic cell, the cell may be any eukaryotic cell which, when the transgene of the invention is introduced therein, and the protein encoded by the desired gene is no longer expressed therefrom, a benefit is obtained. Such a benefit may include the fact that there has been provided a system in which lack of expression of the desired gene can be studied in vitro in the laboratory or in a mammal in which the cell resides, a system wherein cells comprising the introduced gene deletion can be used as research, diagnostic and therapeutic tools, and a system wherein animal models are generated which are useful for the development of new diagnostic and therapeutic tools for selected disease states in a mammal including, for example, constrictive remodeling, arterial restenosis, and the like. That is, one skilled in the art would appreciate, based upon the disclosure provided herein, that because proliferation of fibroblasts with scar tissue formation is part of any would healing process, selected disease states or processes associated with such proliferation that can be investigated by assessing CTHRC1 expression include, but are not limited to, wound healing, bone formation, bone fracture healing, and fibrosis of any organ.

Alternatively, the invention includes a eukaryotic cell which, when the transgene of the invention is introduced therein, and the protein encoded by the desired gene is expressed therefrom where it was not previously present or expressed in the cell or where it is now expressed at a level or under circumstances different than that before the transgene was introduced, a benefit is obtained. Such a benefit may include the fact that there has been provided a system in the expression of the desired gene can be studied in vitro in the laboratory or in a mammal in which the cell resides, a system wherein cells comprising the introduced gene can be used as research, diagnostic and therapeutic tools, and a system wherein animal models are generated which are useful for the development of new diagnostic and therapeutic tools for selected disease states in a mammal.

Such cell expressing an isolated nucleic acid encoding CTHRC1 can be used to provide CTHRC1 to a cell, tissue, or whole animal where a controlled level of CTHRC1 can be useful to treat or alleviate a disease, disorder or condition associated with low level of CTHRC1 expression and/or activity. Such diseases, disorders or conditions can include, but are not limited to, wound healing, bone formation, and bone fracture healing, and the like. Moreover, one skilled in the art would understand that one goal of a wound healing response is to regain mechanical strength and structural support. CTHRC1 is expressed during such a healing response. Controlled levels of expression of CTHRC1 could thus lead to accelerated wound healing, bone growth, and fracture healing. Therefore, the invention includes a cell expressing CTHRC1 to increase or induce CTHR1 expression, translation, and/or activity, where controlling CTHRC1 expression, protein level, and/or activity can be useful to treat or alleviate a disease, disorder or condition.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding CTHRC1 and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all (i.e., a “knock-out” vector) or which insert (i.e., a “knock-in” vector) a nucleic acid encoding CTHRC1, or a fragment thereof, from or into a mammalian genome, respectively. The crucial feature of the targeting vector is that it comprise sufficient portions of two sequences located towards opposite, i.e., 5′ and 3′, ends of the CTHRC1 open reading frame (ORF) in the case of a “knock-out” vector, to allow deletion/insertion by homologous recombination to occur such that all or a portion of the nucleic acid encoding CTHRC1 is deleted from or inserted into a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the CTHRC1 coding region to be used in the targeting vector may be easily selected based upon known methods and following the teachings disclosed herein based on the disclosure provided herein including the nucleic and amino acid sequences of both rat and human CTHRC1. Armed with these sequences, one of ordinary skill in the art would be able to construct the transgenes and knock-out vectors of the invention.

The invention further includes a knock-out targeting vector comprising a nucleic acid encoding a selectable marker such as, for example, a nucleic acid encoding the neoR gene thereby allowing the selection of transgenic a cell where the nucleic acid encoding CTHRC1, or a portion thereof, has been deleted and replaced with the neomycin resistance gene by the cell's ability to grow in the presence of G418. However, the present invention should not be construed to be limited to neomycin resistance as a selectable marker. Rather, other selectable markers well-known in the art may be used in the knock-out targeting vector to allow selection of recombinant cells where the CTHRC1 gene has been deleted and/or inactivated and replaced by the nucleic acid encoding the selectable marker of choice. Methods of selecting and incorporating a selectable marker into a vector are well-known in the art and are describe in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

As noted herein, the invention includes a non-human transgenic mammal comprising an exogenous nucleic acid inserted into a desired site in the genome thereof thereby deleting the coding region of a desired endogenous target gene, i.e., a knock-out transgenic mammal. Further, the invention includes a transgenic non-human mammal wherein an exogenous nucleic acid encoding CTHRC1 is inserted into a site the genome, i.e., a “knock-in” transgenic mammal. The knock-in transgene inserted may comprise various nucleic acids encoding, for example, a tag polypeptide, a promoter/regulatory region operably linked to the nucleic acid encoding CTHRC1 not normally present in the cell or not typically operably linked to CTHRC1.

The generation of the non-human transgenic mammal of the invention is preferably accomplished using the method which is now described. However, the invention should in no way be construed as being limited solely to the use of this method, in that, other methods can be used to generate the desired knock-out mammal.

In the preferred method of generating a non-human transgenic mammal, ES cells are generated comprising the transgene of the invention and the cells are then used to generate the knock-out animal essentially as described in Nagy and Rossant (1993, In: Gene Targeting, A Practical Approach, pp.146-179, Joyner ed., IRL Press). ES cells behave as normal embryonic cells if they are returned to the embryonic environment by injection into a host blastocyst or aggregate with blastomere stage embryos. When so returned, the cells have the full potential to develop along all lineages of the embryo. Thus, it is possible, to obtain ES cells, introduce a desired DNA therein, and then return the cell to the embryonic environment for development into mature mammalian cells, wherein the desired DNA may be expressed.

Precise protocols for the generation of transgenic mice are disclosed in Nagy and Rossant (1993, In: Gene Targeting, A Practical Approach, Joyner ed. IRL Press, pp. 146-179) and are therefore not repeated herein. Transfection or transduction of ES cells in order to introduce the desired DNA therein is accomplished using standard protocols, such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York). Preferably, the desired DNA contained within the transgene of the invention is electroporated into ES cells, and the cells are propagated as described in Soriano et al. (1991, Cell 64:693-702).

Introduction of an isolated nucleic acid into the fertilized egg of the mammal is accomplished by any number of standard techniques in transgenic technology (Hogan et al., 1986, Manipulating the Mouse Embryo: A Laboratory Manual, Cold Spring Harbor, N.Y.). Most commonly, the nucleic acid is introduced into the embryo by way of microinjection.

Once the nucleic acid is introduced into the egg, the egg is incubated for a short period of time and is then transferred into a pseudopregnant mammal of the same species from which the egg was obtained as described, for example, in Hogan et al. (1986, Manipulating the Mouse Embryo: A Laboratory Manual, Cold Spring Harbor, N.Y.). Typically, many eggs are injected per experiment, and approximately two-thirds of the eggs survive the procedure. About twenty viable eggs are then transferred into pseudopregnant animals, and usually four to ten of the viable eggs so transferred will develop into live pups.

Any mammalian CTHRC1 gene may be used in the methods described herein to produce a transgenic mammal or a transgenic cell harboring a transgene comprising a deletion of all or part of that mammalian CTHRC1 gene. Preferably, a rodent CTHRC1 gene such as, e.g., rat CTHRC1 (SEQ ID NO:1), encoding rat CTHRC1_{S} (SEQ ID NO:2) and rat CTHRC1_{L} (SEQ ID NO:5), is used, and human CTHRC1 (SEQ ID NO:3), cCTHRC1_{L} (SEQ ID NO:10), and cCTHRC1_{S} (SEQ ID NO:12) gene, is also used.

The transgenic mammal of the invention can be any species of mammal. Thus, the invention should be construed to include generation of transgenic mammals encoding the chimeric nucleic acid, which mammals include mice, hamsters, rats, rabbits, pigs, sheep and cattle. The methods described herein for generation of transgenic mice can be analogously applied using any mammalian species. Preferably, the transgenic mammal of the invention is a rodent and even more preferably, the transgenic mammal of the invention is a mouse. By way of example, Lukkarinen et al. (1997, Stroke 28:639-645), teaches that gene constructs which enable the generation of transgenic mice also enable the generation of other transgenic rodents, including rats. Similarly, nullizygous mutations in a genetic locus of an animal of one species can be replicated in an animal of another species having a genetic locus highly homologous to the first species.

To identify the transgenic mammals of the invention, pups are examined for the presence of the isolated nucleic acid using standard technology such as Southern blot hybridization, PCR, and/or RT-PCR. Expression of the nucleic acid in the cells and in the tissues of the mammal is also assessed using ordinary technology described herein. Further, the presence or absence of CTHRC1 in the circulating blood of the transgenic animal can be determined, if the protein is secreted, by using, for example, Western blot analysis, or using standard methods for protein detection that are well-known in the art.

Cells obtained from the transgenic mammal of the invention, which are also considered “transgenic cells” as the term is used herein, encompass such as cells as those obtained from the CTHRC1 (±) and (−/−) transgenic non-human mammal described elsewhere herein, are useful systems for modeling diseases and symptoms of mammals which are believed to be associated with altered levels of CTHRC1 expression such as constrictive remodeling, arterial restenosis, adventitial fibrosis, wound healing, bone formation, bone density, dorsal closure, spina bifida-like conditions, and any other disease, disorder or condition associated with an altered level of CTHRC1 expression. Moreover, as a marker of a pathway(s) associated with cell proliferation and cell migration, CTHRC1 expression levels are also useful indicators in assessment of various diseases, disorders or conditions associated with excessive or impaired wound healing (e.g., skin wound healing) and conditions associated with excessive or impaired bone formation, and the like.

Particularly suitable are cells derived from a tissue of the non-human knock-out or knock-in transgenic mammal described herein, wherein the transgene comprising the CTHRC1 gene is expressed or inhibits expression of CTHRC1 in various tissues. By way of example, cell types from which such cells are derived include fibroblasts and like cells of (1) the CTHRC1 (+/+), (±) and (−/−) non-human transgenic liveborn mammal, (2) the CTHRC1 (+/+), (−/−) or (±) fetal animal, and (3) placental cell lines obtained from the CTHRC1 (+/+), (−/−) and (±) fetus and liveborn mammal.

One skilled in the art would appreciate, based upon this disclosure, that cells comprising decreased levels of CTHRC1 protein, decreased level of CTHRC1 activity, or both, include, but are not limited to, cells expressing inhibitors of CTHRC1 expression (e.g., antisense or ribozyme molecules).

Methods and compositions useful for maintaining mammalian cells in culture are well known in the art, wherein the mammalian cells are obtained from a mammal including, but not limited to, cells obtained from a mouse such as the transgenic mouse described herein.

The recombinant cell of the invention can be used to study the effect of qualitative and quantitative alterations in CTHRC1 levels on cell signal transduction systems. This is because the fact that the data disclosed herein indicate that CTHRC1 is involved in TGF-β signaling pathways. Further, the recombinant cell can be used to produce CTHRC1 for use for therapeutic and/or diagnostic purposes. That is, a recombinant cell expressing CTHRC1 can be used to produce large amounts of purified and isolated CTHRC1 that can be administered to treat or alleviate a disease, disorder or condition associated with or caused by a decreased level of CTHRC1.

Alternatively, recombinant cells expressing CTHRC1 can be administered in ex vivo and in vivo therapies where administering the recombinant cells thereby administers the protein to a cell, a tissue, and/or an animal. Additionally, the recombinant cells are useful for the discovery of CTHRC1 ligand(s) and CTHRC1 signaling pathway(s).

The recombinant cell of the invention may be used to study the effects of elevated or decreased CTHRC1 levels on cell homeostasis and cell proliferation and/or migration since CTHRC1 has been demonstrated, by the data disclosed herein, to play a role in cell migration, fibrosis, arterial restenosis, constrictive remodeling, and the like.

The recombinant cell of the invention, wherein the cell has been engineered such that it does not express CTHRC1, or expresses reduced or altered CTHRC1 lacking biological activity, can also be used in ex vivo and in vivo cell therapies where either an animal's own cells (e.g., fibroblasts, and the like), or those of a syngeneic matched donor, are recombinantly engineered as described elsewhere herein (e.g., by insertion of an antisense nucleic acid or a knock-out vector such that CTHRC1 expression and/or protein levels are thereby reduced in the recombinant cell), and the recombinant cell is administered to the recipient animal. In this way, recombinant cells that express CTHRC1 at a reduced level can be administered to an animal whose own cells express increased levels of CTHRC1 thereby treating or alleviating a disease, disorder or condition associated with or mediated by increased CTHRC1 expression as disclosed elsewhere herein.

The transgenic mammal of the invention, rendered susceptible to adventitial fibrosis, arterial restenosis, and the like, such as, for example, a CTHRC1 knock-out mouse, can be used to study the pathogenesis of these diseases and the potential role of CTHRC1 therein.

Further, the transgenic mammal and/or cell of the invention may be used to further study the subcellular localization of CTHRC1.

Also, the transgenic mammal (both ± and −/− live born and fetuses) and/or cell of the invention may be used to study to role(s) of CTHRC1 in cell migration and proliferation, and TGF-β signaling to elucidate the target(s) of CTHRC1 action as well as any receptor(s) and/or ligands that bind with CTHRC1 to mediate its effect(s) in the cell.

VI. Antibodies

The invention also includes an antibody that specifically binds CTHRC1, or a fragment thereof.

One skilled in the art would understand, based upon the disclosure provided herein, that an antibody that specifically binds CTHRC1, binds with a protein of the invention, such as, but not limited to rat CTHRC1s, human CTHRC1, and rat CTHRC1_{L}, or an immunogenic portion thereof. In one embodiment, the antibody is directed to rat CTHRC1 comprising the amino acid sequence of SEQ ID NO:2 and SEQ ID NO:5, and an antibody directed to human CTHRC1, comprising the amino acid sequence SEQ ID NO:4, SEQ ID NO:11, and SEQ ID NO:13.

Polyclonal antibodies are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the CTHRC1 portion is rendered immunogenic (e.g., CTHRC1 conjugated with keyhole limpet hemocyanin, KLH) and a portion comprising the respective rodent and/or human CTHRC1 amino acid residues. The chimeric proteins are produced by cloning the appropriate nucleic acids encoding CTHRC1 (e.g., SEQ ID NO:1, SEQ ID NO:3, SEQ ID NO:10, SEQ ID NO:12) into a plasmid vector suitable for this purpose, such as but not limited to, pMAL-2 or pCMX.

However, the invention should not be construed as being limited solely to these antibodies or to these portions of the protein antigens. Rather, the invention should be construed to include other antibodies, as that term is defined elsewhere herein, to rat and human CTHRC1, or portions thereof. Further, the present invention should be construed to encompass antibodies, inter alia, bind with CTHRC1 and they are able to bind CTHRC1 present on Western blots, in immunohistochemical staining of tissues thereby localizing CTHRC1 in the tissues, and in immunofluorescence microscopy of a cell transiently transfected with a nucleic acid encoding at least a portion of CTHRC1.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian CTHRC1. That is, the invention includes immunizing an animal using an immunogenic portion, or antigenic determinant, of the CTHRC1 protein. Such immunogenic portions can include, but are not limited to, the carboxy-terminal 15 amino acids (GWNSVSRIIIEELPK) (SEQ ID NO:7). The antibodies can be produced by immunizing an animal such as, but not limited to, a rabbit or a mouse, with a protein of the invention, or a portion thereof, or by immunizing an animal using a protein comprising at least a portion of CTHRC1, or a fusion protein including a tag polypeptide portion comprising, for example, a maltose binding protein tag polypeptide portion covalently linked with a portion comprising the appropriate CTHRC1 amino acid residues. One skilled in the art would appreciate, based upon the disclosure provided herein, that smaller fragments of these proteins can also be used to produce antibodies that specifically bind CTHRC1.

One skilled in the art would appreciate, based upon the disclosure provided herein, that various portions of an isolated CTHRC1 polypeptide can be used to generate antibodies to either highly conserved regions of CTHRC1 or to non-conserved regions of the polypeptide. As disclosed elsewhere herein, CTHRC1 comprises various conserved domains including, but not limited to, a putative signal peptide from about amino acid residue 1 to about amino acid residue 32 transmembrane domain/signal peptide (amino acid residues from about 1 to 32); a CK2 phosphorylation domain (amino acid residues from about 31 to 34); an N-myristoylation domain (amino acid residues from about 69 to 74); a collagen domain (amino acid residues from about aa 60 to 95); a CK2 phosphorylation domain (amino acid residues from about 99 to 102); an N-myristoylation domain (amino acid residues from about 119 to 124); a PKC phosphorylation domain (amino acid residues from about 146 to 148); an N-myristoylation domain (amino acid residues from about 165 to 170); an N-glycosylation domain (amino acid residues from about 188 to 191); a CK2 phosphorylation domain (amino acid residues from about 197 to 200); an N-myristoylation domain (amino acid residues from about 201 to 206); an N-myristoylation domain (amino acid residues from about 205 to 210); and a CK2 phosphorylation domain (amino acid residues from about 219 to 222). These domains are also present in rat and human CTHRC1s (see, e.g., SEQ ID NO:2, SEQ ID NO:4, SEQ ID NO:5, SEQ ID NO:11, and SEQ ID NO:13).

Once armed with the sequence of CTHRC1 and the detailed analysis localizing the various conserved and non-conserved domains of the protein, the skilled artisan would understand, based upon the disclosure provided herein, how to obtain antibodies specific for the various portions of a mammalian CTHRC1 polypeptide using methods well-known in the art or to be developed, as well as methods disclosed herein.

Further, the skilled artisan, based upon the disclosure provided herein, would appreciate that the non-conserved regions of a protein of interest can be more immunogenic than the highly conserved regions which are conserved among various organisms. Further, immunization using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of each CTHRC1 molecule can be used to produce antibodies that are specific only for that CTHRC1 and do not cross-react non-specifically with other CTHRC1 s or with other proteins.

Alternatively, the skilled artisan would also understand, based upon the disclosure provided herein, that antibodies developed using a region that is conserved among one or more CTHRC1 molecule can be used to produce antibodies that react specifically with one or more CTHRC1 molecule. Methods for producing antibodies that specifically bind with a conserved protein domain which may otherwise be less immunogenic than other portions of the protein are well-known in the art and include, but are not limited to, conjugating the protein fragment of interest to a molecule (e.g., keyhole limpet hemocyanin, and the like), thereby rendering the protein domain immunogenic, or by the use of adjuvants (e.g., Freund's complete and/or incomplete adjuvant, and the like), or both. Thus, the invention encompasses antibodies that recognize at least one CTHRC1 and antibodies that specifically bind with more than one CTHRC1, including antibodies that specifically bind with all CTHRC1.

One skilled in the art would appreciate, based upon the disclosure provided herein, which portions of CTHRC1 are less homologous with other proteins sharing conserved domains. However, the present invention is not limited to any particular domain; instead, the skilled artisan would understand that other non-conserved regions of the CTHRC1 proteins of the invention can be used to produce the antibodies of the invention as disclosed herein.

Therefore, the skilled artisan would appreciate, based upon the disclosure provided herein, that the present invention encompasses antibodies that neutralize and/or inhibit CTHRC1 activity (e.g., by inhibiting necessary CTHRC1 receptor/ligand interactions, by inhibiting triple helix formation, and the like), which antibodies can recognize one or more CTHRC1 s, including, but not limited to, rat CTHRC1 s, rat CTHRC1_{L}, and human CTHRC1 (e.g., full-length, cleaved longer fragment, cleaved shorter fragment, CTHRC1 wherein certain domains have been deleted and/or altered, among others), as well as CTHRC1 s from various species (e.g., mouse CTHRC1).

The invention should not be construed as being limited solely to the antibodies disclosed herein or to any particular immunogenic portion of the proteins of the invention. Rather, the invention should be construed to include other antibodies, as that term is defined elsewhere herein, to CTHRC1, or portions thereof, or to proteins sharing at least about 6% homology with a polypeptide having the amino acid sequence of at least one of SEQ ID NO:2, SEQ ID NO:4, SEQ ID NO:5, SEQ ID NO:11, and SEQ ID NO:13. Preferably, the polypeptide is about 10% homologous, more preferably, at least about 15% homologous, more preferably, at least about 20% homologous, even more preferably, at least about 25% homologous, more preferably, at least about 30% homologous, preferably, at least about 35% homologous, even more preferably, at least about 40% homologous, even more preferably, at least about 45% homologous, yet more preferably, at least about 50% homologous, more preferably, at least about 55% homologous, more preferably, at least about 60% homologous, even more preferably, at least about 65% homologous, yet more preferably, at least about 70% homologous, more preferably, at least about 75% homologous, even more preferably, at least about 80% homologous, yet more preferably, at least about 85% homologous, more preferably, at least about 90% homologous, even more preferably, at least about 95% homologous, and most preferably, at least about 99% homologous to at least one of rat CTHRC1 s (SEQ ID NO:2), rat CTHRC1_{L} (SEQ ID NO:5), human CTHRC1 (SEQ ID NO:4), human cCTHRC1_{L} (SEQ ID NO:11), and human cCTHRC1_{S} (SEQ ID NO:13). More preferably, the polypeptide that specifically binds with an antibody specific for mammalian CTHRC1 is at least one of rat CTHRC1_{S} (SEQ ID NO:2), rat CTHRC1_{L} (SEQ ID NO:5), human CTHRC1 (SEQ ID NO:4), human cCTHRC1_{L} (SEQ ID NO:11), and human cCTHRC1_{S} (SEQ ID NO: 13). Most preferably, the polypeptide that specifically binds with an antibody that specifically binds with a mammalian CTHRC1 is at least one of SEQ ID NO: 2, SEQ ID NO:4, SEQ ID NO:5, SEQ ID NO:11, and SEQ ID NO:13.

Further, the skilled artisan would appreciate, based upon the disclosure provided herein, that amino acid sequences that may elicit antibodies that non-specifically cross-react with a non-CTHRC1 protein can also be excluded from use as immunogens. For example, such amino acid sequences include, but are not limited to, an amino acid sequence comprising collagen alpha-2 (IV) chain precursor (GenBank Acc. No. P27393), which shares about 62% identity with CTHRC1 over a 35 amino acid stretch. Thus, such a portion sharing at least about 62% identity over 35 amino acids of CTHRC1 would not be used to produce the antibodies of the invention.

The invention encompasses polyclonal, monoclonal, synthetic antibodies, and the like. One skilled in the art would understand, based upon the disclosure provided herein, that the crucial feature of the antibody of the invention is that the antibody bind specifically with CTHRC1. That is, the antibody of the invention recognizes CTHRC1, or a fragment thereof (e.g., an immunogenic portion or antigenic determinant thereof), as demonstrated by antibody binding CTHRC1 on Western blots, in immunostaining of cells, and/o immunoprecipitation of CTHRC1, using standard methods well-known in the art.

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art.

In addition, the antibody can be used to decrease the level of CTHRC1 in a cell thereby inhibiting the effect(s) of CTHRC1 in a cell. Thus, by administering the antibody to a cell or to the tissues of an animal or to the animal itself, the required CTHRC1 receptor/ligand interactions are therefore inhibited such that the effect of CTHRC1-mediated signaling are also inhibited. One skilled in the art would understand, based upon the disclosure provided herein, that detectable effects upon altering CTHRC1 ligand/receptor interaction using an anti-CTHRC1 antibody can include, but are not limited to, manipulating proliferation of cells, cell migration, constrictive remodeling, adventitial fibrosis, arterial restenosis, fibrosis in any organ or tissue, ossification or bone formation, and the like.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (supra), and in the references cited therein, and in Gu et al. (1997, Thrombosis and Hematocyst 77:755-759), and other methods of humanizing antibodies well-known in the art or to be developed.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

VII. Compositions

The invention includes a composition comprising an isolated nucleic acid complementary to a nucleic acid, or a portion thereof, encoding a mammalian CTHRC1, which is in an antisense orientation with respect to transcription. Preferably, the composition comprises a pharmaceutically acceptable carrier.

The invention includes a composition comprising an isolated mammalian CTHRC1 polypeptide as described herein. Preferably, the composition comprises a pharmaceutically-acceptable carrier.

The invention also includes a composition comprising an antibody that specifically binds CTHRC1. Preferably, the composition comprises a pharmaceutically-acceptable carrier.

The invention further includes a composition comprising an isolated nucleic acid encoding a mammalian CTHRC1. Preferably, the composition comprises a pharmaceutically acceptable carrier.

The compositions can be used to administer CTHRC1 to a cell, a tissue, or an animal or to inhibit expression of CTHRC1 in a cell, a tissue, or an animal. The compositions are useful to treat a disease, disorder or condition mediated by altered expression of CTHRC1 such that decreasing or increasing CTHRC1 expression or the level of the protein in a cell, tissue, or animal, is beneficial to the animal. That is, where a disease, disorder or condition in an animal is mediated by or associated with altered level of CTHRC1 expression or protein level, the composition can be used to modulate such expression or protein level of CTHRC1.

For administration to the mammal, a polypeptide, or a nucleic acid encoding it, and/or an antisense nucleic acid complementary to all or a portion thereof, can be suspended in any pharmaceutically acceptable carrier, for example, HEPES buffered saline at a pH of about 7.8.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer CTHRC1 and/or a nucleic acid encoding the same according to the methods of the invention.

Compounds which are identified using any of the methods described herein may be formulated and administered to a mammal for treatment of arterial restenosis, adventitial fibrosis, fibrosis in any organ or tissue, negative remodeling, excessive bone formation, excessive ossification, any condition associated with, or mediated by, collagen matrix formation, and the like, are now described.

The invention encompasses the preparation and use of pharmaceutical compositions comprising a compound useful for treatment of arterial restenosis, adventitial fibrosis, negative remodeling, and the like, as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than I nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

VIII. Methods

A. Methods of Identifying Useful Compounds

The present invention further includes a method of identifying a compound that affects expression of CTHRC1 in a cell. The method comprises contacting a cell with a test compound and comparing the level of expression of CTHRC1 in the cell so contacted with the level of expression of CTHRC1 in an otherwise identical cell not contacted with the compound. If the level of expression of CTHRC1 is higher or lower in the cell contacted with the test compound compared to the level of expression of CTHRC1 in the otherwise identical cell not contacted with the test compound, this is an indication that the test compound affects expression of CTHRC1 in a cell.

Similarly, the present invention includes a method of identifying a compound that reduces expression of CTHRC1 in a cell. The method comprises contacting a cell with a test compound and comparing the level of expression of CTHRC1 in the cell contacted with the compound with the level of expression of CTHRC1 in an otherwise identical cell, which is not contacted with the compound. If the level of expression of CTHRC1 is lower in the cell contacted with the compound compared to the level in the cell that was not contacted with the compound, then that is an indication that the test compound affects reduced expression of CTHRC1 in a cell.

One skilled in the art would appreciate, based on the disclosure provided herein, that the level of expression of CTHRC1 in the cell can be measured by determining the level of expression of mRNA encoding CTHRC1. Alternatively, the level of expression of mRNA encoding CTHRC1 can be determined by using immunological methods to assess CTHRC1 production from such mRNA as exemplified herein using Western blot analysis using an anti-CTHRC1 antibody of the invention. Further, nucleic acid-based detection methods, such as Northern blot and PCR assays and the like, can be used as well. In addition, the level of CTHRC1 activity in a cell can also be assessed by determining the level of various parameters which can be affected by CTHRC1 activity such as, for example, the level of cell proliferation and/or migration, the level of expression in adventitia, the level of adventitial fibrosis, the level of fibrosis in other organs (e.g., lung, liver, among others), the level of arterial restenosis, the level of ossification, the level of bone formation and fracture healing, the level of osteoblast proliferation, and the like. Thus, one skilled in the art would appreciate, based upon the extensive disclosure and reduction to practice provided herein, that there are a plethora of methods which can be used to asses the level of expression of CTHRC1 in a cell including those methods disclosed herein, methods well-known in the art, and other methods to be developed in the future.

Further, one skilled in the art would appreciate based on the disclosure provided herein that, as disclosed in the examples below, a cell which lacks endogenous CTHRC1 expression can be transfected with a vector comprising an isolated nucleic acid encoding CTHRC1 whereby expression of CTHRC1 is effected in the cell. The transfected cell is then contacted with the test compound thereby allowing the determination of whether the compound affects the expression of CTHRC1. Therefore, one skilled in the art armed with the present invention would be able to, by selectively transfecting a cell lacking detectable levels of CTHRC1 using CTHRC1-expressing vectors, identify a compound which selectively affects CTHRC1 expression.

The skilled artisan would further appreciate, based upon the disclosure provided herein, that where an isolated nucleic acid encoding CTHRC1 is administered to a cell lacking endogenous detectable levels of CTHRC1 expression such that detectable CTHRC1 is produced by the cell, the isolated nucleic acid can comprise an additional nucleic acid encoding, e.g., a tag polypeptide, covalently linked thereto. This allows the detection of CTHRC1 expression by detecting the expression of the tag polypeptide. Thus, the present invention encompasses methods of detecting CTHRC1 expression by detecting expression of another molecule which is co-expressed with CTHRC1.

One skilled in the art would understand, based upon the disclosure provided herein, that the invention encompasses administering a nucleic acid to a cell wherein the nucleic acid is expressed and thereby reduces the level of CTHRC1 in the cell. That is, as disclosed elsewhere herein, the invention encompasses a cell wherein the level of CTHRC1 (either transcription, translation, or both) is inhibited. While such a cell is exemplified elsewhere herein by a cell transfected with an antisense nucleic acid complementary to a nucleic acid encoding CTHRC1, or a portion thereof, the present invention is not limited to this, or any other particular, method for inhibiting expression of a nucleic acid in a cell.

The invention includes a method of identifying a protein that specifically binds with CTHRC1. That is, one skilled in the art would appreciate, based upon the disclosure provided herein, that CTHRC1, which comprises several myristoylation domains. Further, CTHRC1 comprises a putative signal peptide indicating the molecule can be secreted from a cell. These data indicate that CTHRC1 likely effects its biological function(s) by specifically binding with at least one protein, preferably a CTHRC1 receptor, another CTHRC1 molecule, and/or a CTHRC1 ligand. Thus, the invention encompasses methods, which are well-known in the art or to be developed, for identifying a protein that specifically binds with and/or associates with CTHRC1. Such methods include, but are not limited to, protein binding assays wherein the target protein, i.e., CTHRC1, is immobilized on an appropriate support and incubated under conditions that allow CTHRC1 binding with a CTHRC1-associated protein. CTHRC1 can be immobilized on a support using standard methods such as, but not limited to, production of CTHRC1 comprising a glutathione-S-transferase (GST) tag, a maltose binding protein (MBP) tag, or a His₆-tag, where the fusion protein is then bound to glutathione-Sepharose beads, a maltose-column, or a nickel chelation resin (e.g., His-Bind resin, Novagen, Madison, Wis.), respectively. The solid support is washed to remove proteins which may be non-specifically bound thereto and any CTHRC1 -associated protein can then be dissociated from the matrix thereby identifying a CTHRC1-associated protein.

In addition, a protein that specifically binds with CTHRC1, e.g., a receptor, a ligand, and/or other CTHRC1 -associated protein, can be identified using, for example, a yeast two hybrid assay. Yeast two hybrid assay methods are well-known in the art and can be performed using commercially available kits (e.g., MATCHMAKER™ Systems, Clontech Laboratories, Inc., Palo Alto, Calif., and other such kits) according to standard methods. Therefore, once armed with the teachings provided herein, e.g., the full amino and nucleic acid sequences of the “bait” protein, CTHRC1, one skilled in the art can easily identify a protein that specifically binds with CTHRC1 such as, but not limited to, a CTHRC1 receptor protein, a CTHRC1 ligand, and the like.

One skilled in the art would understand, based upon the disclosure provided herein, that the invention encompasses any molecule identified using the methods discussed elsewhere herein. That is, molecules that associate with CTHRC1, such as but not limited to, a CTHRC1 receptor protein, a CTHRC1 ligand protein, or both, can be used to develop therapeutics and diagnostics for diseases, disorders or conditions mediated by CTHRC1 interaction with a CTHRC1 -associated protein such as constrictive remodeling, arterial restenosis, adventitial fibrosis, excessive wound healing responses, scarring, keloids, excessive bone formation, fracture healing, ectopic ossification, excessive fibrous tissue formation, failure of dorsal closure, spina bifida-like effects, and the like. That is, one skilled in the art would appreciate, as more fully set forth elsewhere herein in discussing antibodies that specifically bind with CTHRC1, that a CTHRC1-associated protein can be used to develop therapeutics that inhibit CTHRC1 activity in a cell by inhibiting necessary CTHRC1 receptor/ligand interactions and other CTHRC1 binding interactions, which are required for CTHRC1 activity.

CTHRC1 -associated proteins identified by the above-disclosed methods can be used directly to inhibit CTHRC1 interactions by contacting a cell with the CTHRC1 -associated protein, or a portion thereof, or they can be used to develop antibodies and/or peptidomimetics that can inhibit the CTHRC1 -associated interaction with CTHRC1 thereby inhibiting CTHRC1 function and activity. Thus, CTHRC1-associated proteins, including a CTHRC1 receptor/ligand protein, are useful and are encompassed by the invention.

B. Methods of Treating or Alleviating a Disease, Disorder or Condition Associated with or Mediated by CTHRC1 Expression

The data disclosed herein demonstrate that CTHRC1 expression is induced in fibroblasts following an injury. As such, CTHRC1 is part of any wound healing process which is characterized by granulation tissue formation, proliferation, and migration of fibroblasts with subsequent apoptosis of these cells extracellular matrix production. Although the wound healing response/process is a normal physiologic response to injury, there are many conditions where an excessive wound healing response leads to symptoms or disease. Thus, the skilled artisan would appreciate, based upon the disclosure provided herein, that because the wound healing response is similar for all organs and tissues, the effects of expression of CTHRC1 are similar, independent of where the injury occurred. Excessive wound healing would result in various conditions depending on the situs of injury such that, for example, in an artery, excessive wound healing would result in constrictive remodeling with loss of vessel diameter due to adventitial fibrosis. The formation of scars and keloids is an excessive fibrotic reaction associated with excessive wound healing. Chronic inflammatory conditions often lead to organ fibrosis, e.g., liver fibrosis and lung fibrosis.

One skilled in the art, armed with the teachings provided herein, would appreciate that the invention encompasses methods for treating and/or alleviating a disease mediated by, or associated with, collagen matrix production. The method comprises increasing the level of CHTRC1 in a cell, organ, or tissue. This is because the data disclosed elsewhere herein demonstrate that an increased level of CTHRC1 decreases the production of collagen matrix and, thereby, inhibits, among other things, restenosis in a vessel following, inter alia, an injury, renal fibrosis, and the like. This is because it is known that restenosis is largely caused by constrictive remodeling, due to fibrosis of the adventitia (see, e.g., Smith et al., 1999, Circ. Res. 84:1212-1222). Since, as demonstrated by the data disclosed elsewhere herein, overexpression of CTHRC1 inhibited fibrotic disease in an art-recognized model of renal fibrosis, as well as in a model of arterial restenosis, an increased level of CTHRC1 can decrease constrictive remodeling and prevent or inhibit restenosis following arterial injury, such as the injury associated with angioplasty. Thus, the present invention includes a method of preventing, or treating a disease or disorder mediated by fibrosis related to collagen matrix formation by administering CTHRC1.

The skilled artisan, based upon the disclosure provided herein, would appreciate that administering CTHRC1 encompasses any method of providing CTHRC1 to a cell, tissue or organ. That is, the invention includes, but is not limited to, administering a CTHRC1 polypeptide, a nucleic acid encoding CTHRC1, or any other means of providing CHTRC1 to a cell, such as, but not limited to, gene activation methods whereby an endogenous nucleic acid encoding CTHRC1 is activated such that it is expressed at a higher level than before gene activation was performed, and/or where CTHRC1 was not expressed at a detectable level prior to activation, but is expressed at a detectable level upon such activation.

Accordingly, the invention includes a method of treating a disease mediated by collagen matrix production in a human. That is, a human afflicted with a disease mediated by, or associated with, production of collagen matrix can be treated by CTHRC1 administered to the human. This is because the data disclosed herein demonstrate that expression of CTHRC1 mediates a decrease in collagen matrix deposition and thereby provides a therapeutic effect in various art-recognized models for human diseases mediated by, or associated with, collagen matrix deposition. More specifically, the data demonstrate that renal fibrosis and arterial restenosis can be treated and/or prevented by administration of CTHRC1. While the diseases are exemplified by restenosis and fibrosis, the skilled artisan would appreciated, based upon the disclosure provided herein, that the present invention is in no way limited to these particular diseases. Rather, where a disease or condition is associated with, or mediated by collagen matrix production, one skilled in the art, once armed with the teachings provided herein, would readily appreciate that such condition or disease can be treated, prevented, or both, by administration of CTHRC1.

Further, methods of administering a therapeutic compound, such as CTHRC1, are well-known in the art and are, inter alia, discussed elsewhere herein. That is, the dose, route of administration, treatment regimen and the like, are well understood and depend upon, among other factors, the disease site, the age, size, and state of the human, as well as the state and stage of the disease to be treated. Thus, even though the data disclosed herein demonstrate administration of CTHRC1 comprising administering a nucleic acid encoding CTHRC1 to a cell where it is expressed, and where the cell is present in a transgenic mammal, the invention is not limited to this, or any other, method for administering CTHRC1. Instead, the skilled artisan would appreciate, based upon the disclosure provided herein, that any method for administering a protein to a cell, organ, tissue, or organism, including, but not limited to administering the protein, a nucleic acid encoding it, or any other method either known in the art or to be developed in the future, is included in the present invention.

Similarly, while CTHRC1 can be administered systemically, the present invention is not limited to this, or any other, route of administration. Thus, as more fully discussed elsewhere herein with respect to pharmaceutical compositions, CTHRC1 can be administered to a human using a plethora of methods, including, but not limited to, implanting a device comprising CTHRC1, such that the protein is delivered to a specific site. This can be useful for, among other things, administering CTHRC1 to a wound, or to a site of vessel injury (e.g., associated with angioplasty, and the like), to inhibit or reduce collagen matrix production at the site of interest. This would be well understood by the skilled artisan armed with the teachings provided herein and is, therefore, not discussed further.

In another aspect, the invention includes a method of treating constrictive remodeling in a human. This is because, as amply demonstrated by the data disclosed herein, administration of CTHRC1 to a cell, tissue or organ prevents, reduces and/or inhibits production of collagen matrix, which, in turn inhibits or decreases constrictive remodeling that would otherwise result from such collagen matrix production.

Likewise, the invention encompasses a method of preventing constrictive remodeling in a human. That is because the data disclosed herein demonstrate, for the first time, that administration of CTHRC1 to a cell, organ, tissue, or organism can prevent constrictive remodeling in an art-recognized model. Thus, by administering CTHRC1 before or during collagen matrix deposition, such deposition can be prevented, thereby preventing the constrictive remodeling associated with the collagen matrix production. The present invention therefore provides important novel therapeutics for treatment and prevention of constrictive remodeling.

The invention also provides a method of treating restenosis in a human. The method comprises administering CTHRC1 (e.g., a CTHRC1 protein, a nucleic acid encoding CTHRC1, and the like) to a human afflicted with restenosis, This is because the data disclosed herein demonstrate that administration of CTHRC1 inhibits collagen matrix production thereby inhibiting restenosis mediated by such collagen matrix production.

In addition, the invention encompasses a method of inhibiting restenosis in a human. This is because the data disclosed herein that administration of CTHRC1 in an art-recognized model of restenosis inhibited restenosis following vessel injury, such as that caused by angioplasty. The skilled artisan would appreciate that the present invention is not limited to any particular restenosis, only that the restenosis be mediated by collagen matrix deposition. Therefore, the present invention provides important novel therapeutics for treatment, prevention, or both, of restenosis, which is a common and serious complication following vessel injury by, among other things, angioplasty.

The present invention also provides a method of treating fibrosis in a human. The method comprises administering CTHRC1 to a human afflicted by fibrosis thereby treating the disease. This is because, as amply demonstrated by the data disclosed herein, administration of CTHRC1 prevents or inhibits formation of collagen matrix and thereby inhibits, prevents, and/or reduces fibrosis associated with such collagen matrix deposition. Moreover, although the data disclosed herein demonstrate inhibition of renal fibrosis, the present invention is not limited to inhibition of renal fibrosis. Rather, administration of CTHRC1 can inhibit a plethora of organ and tissue fibrosis wherein the fibrosis is mediated by collagen matrix production, and the skilled artisan would readily appreciate this based upon the disclosure provided herein.

Furthermore, the invention includes a method of preventing fibrosis in a human. That is, the method comprises administering CTHRC1 to the human before onset of fibrosis mediated by collagen matrix deposition. This is because the data disclosed herein demonstrate that where CTHRC1 was administered before fibrosis was detected in an art-recognized model of organ (e.g., renal) fibrosis, fibrosis was inhibited. Therefore, the present invention provides a method for inhibiting fibrosis in a human, where fibrosis is mediated by collagen matrix production, since the surprising data disclosed herein demonstrate that production of collagen matrix is inhibited by administration of CTHRC1 thereby preventing fibrosis.

In sum, because the data disclosed herein demonstrate the antifibrotic properties of CTHRC1, the data support methods comprising administration of CTHRC1 to a cell, tissue, or organ, to reduce fibrosis and any condition, disorder or disease associated with such fibrosis, where the skilled artisan would understand, based upon the disclosure provided herein, the condition, disorder or condition wherein administration of CTHRC1 to increase the level of CTHRC1 in a cell or tissue can provide a benefit, such as, but not limited to, renal fibrosis, and arterial restenosis following vessel injury. Thus, the skilled artisan would appreciate, based upon the disclosure provided herein, that delivery of CTHRC1, for instance, in concert with implantation of a device, such as a stent, whereby the device is coated with CTHRC1, or a matrix providing for its controlled and/or sustained release, is encompassed in the present invention. Methods relating to implantation of devices comprising a substance that is released therefrom are well-known in the art and are therefore not discussed further herein.

The skilled artisan would understand, based upon the disclosure provided herein, that increasing CTHRC1 expression and/or activity in a cell, tissue or organ can treat a disease, disorder or condition associated with excessive collagen production. This is because the data disclosed herein demonstrate that in certain instances, increasing CTHRC1 expression is therapeutic, e.g., by inhibiting fibrosis.

Whether expression of CTHRC1, levels of the polypeptide, or its activity, is normal or decreased, one skilled in the art would appreciate, based on this disclosure, that methods of inducing CTHRC1 of the invention encompass administering a recombinant cell that expresses CTHRC1. Thus, one skilled in the art would appreciate, based on the disclosure provided herein, that the present invention encompasses cell and gene therapy methods to effect a detectable increase in the level of CTHRC1 expression in a mammal.

Additionally, the invention includes ex vivo techniques where a cell is obtained from the mammal, modified to express increased level of CTHRC1, and reintroduced into the mammal. Moreover, cells from the mammal which express a normal level of CTHRC1, compared with the level of CTHRC1 expressed in an otherwise identical cell obtained from a like mammal, can be grown and expanded and an effective number of the cells can be reintroduced into the mammal. Such methods include cell and gene therapy techniques relating to use of bone marrow stromal cells which methods are well-known in the art. Thus, one skilled in the art would appreciate that cell therapy and gene therapy relating to cells that have or lack detectable CTHRC1 expression wherein the cells are administered in vivo are encompassed in the present invention.

The invention includes a method of affecting cellular gene expression by administering an isolated nucleic acid encoding CTHRC1. This is because, as the data presented herein demonstrate, expression of a nucleic acid encoding CTHRC1 affects the level of several cellular genes including, but not limited to, TGF-β1, collagen type1, osteopontin (OPN), Msx1, biglycan, alkaline phosphatase (ALP), and bone morphogenic proteins 1 (BMP1) and 4 (BMP-4). Thus, increasing the level of CTHRC1 in a cell, such as by, among other things, administering a nucleic acid encoding CTHRC1 to the cell and expressing the CTHRC1 therefrom under the control of a promoter that drives increased level of expression, can be used to control the expression of various proteins, including, but not limited to, TGF-β1, collagen type 1, osteopontin, Msx1, biglycan, ALP, BMP-1, and BMP-4, where affecting the expression of these proteins provides a benefit to a mammal.

In addition to replacing defective cells with repaired cells or normal cells from syngeneic, immunologically-matched donors, the method of the invention may also be used to facilitate expression of a desired protein that when secreted in the an animal, has a beneficial effect. That is, cells may be isolated, furnished with a gene encoding CTHRC1 and introduced into the donor or into a syngeneic matched recipient wherein expression of exogenous CTHRC1 exerts a therapeutic effect.

One skilled in the art would understand, based upon the disclosure provided herein, that secretion of CTHRC1 from a cell is contemplated in the present invention. That is, the routineer would appreciate, based upon the disclosure provided herein, that secretion of CTHRC1 from a cell can be a useful therapeutic method and that the present invention includes secretion of CTHRC1 from a cell. Secretion of CTHRC1 from a cell can be effected according to standard methods well-known in the art and methods to be developed in the future. Such methods include, but are not limited to, covalently linking a nucleic acid encoding a signal peptide of a secreted molecule (e.g., insulin; MALLVHFLPLLALLALWEPKPTQA [SEQ ID NO:8]) to the 5′ end of an isolated nucleic acid encoding CTHRC1. A plethora of signal sequences that can be used to mediate secretion of a protein from a cell are available and well-known in the art and the invention includes those as well as sequences to be developed in the future to drive secretion of a protein from a cell.

This aspect of the invention relates to gene therapy in which therapeutic amounts of CTHRC1 are administered to an individual. That is, according to some aspects of the present invention, recombinant cells transfected with a nucleic acid encoding CTHRC1 can be used as cell therapeutics to treat a disease, disorder or a condition characterized by normal or decreased expression of CTHRC1.

In particular, a gene construct that comprises a heterologous gene which encodes CTHRC1 is introduced into cells. These recombinant cells are used to purify isolated CTHRC1, which was is administered to an animal. One skilled in the art would understand, based upon the disclosure provided herein, that instead of administering an isolated CTHRC1 polypeptide, CTHRC1 can be administered to a mammal in need thereof by administering to the mammal the recombinant cells themselves. This will benefit the recipient individual who will benefit when the protein is expressed and secreted by the recombinant cell into the recipient's system.

According to the present invention, gene constructs comprising nucleotide sequences of the invention are introduced into cells. That is, the cells, referred to herein as “recombinant cells,” are genetically altered to introduce a nucleic acid encoding CTHRC1, thereby mediating a beneficial effect in a recipient to which the recombinant cell is administered. According to some aspects of the invention, cells obtained from the same individual to be treated or from another individual, or from a non-human animal, can be genetically altered to replace a defective CTHRC1 gene and/or to introduce a CTHRC1 gene whose expression has a beneficial effect on the individual.

In some aspects of the invention, an individual suffering from a disease, disorder or a condition can be treated by supplementing, augmenting and/or replacing defective or deficient nucleic acid encoding CTHRC1 by providing an isolated recombinant cell containing gene constructs that include normal, functioning copies of a nucleic acid encoding CTHRC1. This aspect of the invention relates to gene therapy in which the individual is provided with a nucleic encoding CTHRC1 for which they are deficient in presence and/or function. The isolated nucleic acid encoding CTHRC1 provided by the cell compensates for the defective CTHRC1 expression of the individual, because, when the nucleic acid is expressed in the individual, a protein is produced which serves to alleviate or otherwise treat the disease, disorder or condition in the individual. Such nucleic acid preferably encodes a CTHRC1 polypeptide that is secreted from the recombinant cell.

In all cases in which a gene construct encoding CTHRC1 is transfected into a cell, the nucleic acid is operably linked to an appropriate promoter/regulatory sequence which is required to achieve expression of the nucleic acid in the recombinant cell. Such promoter/regulatory sequences include but are not limited to, constitutive and inducible and/or tissue specific and differentiation specific promoters, and are discussed elsewhere herein. Constitutive promoters include, but are not limited to, the cytomegalovirus immediate early promoter and the Rous sarcoma virus promoter. In addition, housekeeping promoters, such as those which regulate expression of housekeeping genes, may also be used. Other promoters include those which are preferentially expressed in cells of the central nervous system, such as, but not limited to, the promoter for the gene encoding glial fibrillary acidic protein. In addition, promoter/regulatory elements may be selected such that gene expression is inducible. For example, a tetracycline inducible promoter may be used (Freundlich et al., 1997, Meth. Enzymol. 283:159-173).

The gene construct is preferably provided as an expression vector which includes the coding sequence of a mammalian CTHRC1 of the invention operably linked to essential promoter/regulatory sequences such that when the vector is transfected into the cell, the coding sequence is expressed by the cell. The coding sequence is operably linked to the promoter/regulatory elements necessary for expression of the sequence in the cells. The nucleotide sequence that encodes the protein may be cDNA, genomic DNA, synthesized DNA or a hybrid thereof or an RNA molecule such as mRNA.

The gene construct, which includes the nucleotide sequence encoding CTHRC1 operably linked to the promoter/regulatory elements, may remain present in the cell as a functioning episomal molecule or it may integrate into the chromosomal DNA of the cell. Genetic material may be introduced into cells where it remains as separate genetic material in the form of a plasmid. Alternatively, linear DNA which can integrate into a host cell chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

In order for genetic material in an expression vector to be expressed, the promoter/regulatory elements must be operably linked to the nucleotide sequence that encodes the protein. In order to maximize protein production, promoter/regulatory sequences may be selected which are well suited for gene expression in the desired cells. Moreover, codons may be selected which are most efficiently transcribed in the cell. One having ordinary skill in the art can produce recombinant genetic material as expression vectors which are functional in the desired cells.

It is also contemplated that promoter/regulatory elements may be selected to facilitate tissue specific expression of the protein. Thus, for example, specific promoter/regulatory sequences may be provided such that the heterologous gene will be expressed only in the tissue where the recombinant cells are implanted. Additionally, the skilled artisan would appreciate, based upon the disclosure provided herein, that the CTHRC1 promoter can be operably linked to a nucleic acid of interest thereby directing the expression of the nucleic acid at the site of tissue or organ injury and wounding. More specifically, the CTHRC1 promoter can be used, but is not limited to, direct expression of an angiogenic growth factor to promote angiogenesis after myocardial infarction. Similarly, the CTHRC1 promoter can drive expression of a nucleic acid of interest where such expression is beneficial where tissue ischemia and impaired wound healing are a problem (e.g., ulcerations of the skin, and the like).

One skilled in the art would understand, based upon the disclosure provided herein, that the preferred tissues where the expression of CTHRC1 is to be increased include, but are not limited to, skin, lung and the like. In addition, promoter/regulatory elements may be selected such that gene expression is inducible. For example, a tetracycline inducible promoter may be used (Freundlich et al., 1997, Meth. Enzymol. 283:159-173).

Without wishing to be bound by any particular theory, the nucleic acid encoding CTHRC1 preferably includes a putative signal sequence as disclosed elsewhere herein (e.g., amino acids 1 to 32 of human CTHRC1; SEQ ID NO:3) and amino acids 1 to 32 of rat CTHRC1_{S} (SEQ ID NO:1), which may direct the transport and secretion of the CTHRC1 encoded by the isolated nucleic acid in the recombinant cell. The signal sequence is likely processed and removed upon secretion of the mature CTHRC1 protein from the cell. Alternatively, without wishing to be bound by any particular theory, the putative signal sequence may not be cleaved, but may instead be a transmembrane domain.

In addition to providing cells with recombinant genetic material that either corrects a genetic defect in the cells, that encodes a protein which is otherwise not present in sufficient quantities and/or functional condition so that the genetic material corrects a genetic defect in the individual, and/or that encodes a protein which is useful as beneficial in the treatment or prevention of a particular disease, disorder or condition associated therewith, genetic material can also be introduced into the recombinant cells used in the present invention to provide a means for selectively terminating such cells, should such termination become desirable. Such means for targeting recombinant cells for destruction may be introduced into recombinant cells.

According to the invention, recombinant cells can be furnished with genetic material which renders them specifically susceptible to destruction. For example, recombinant cells may be provided with a gene that encodes a receptor that can be specifically targeted with a cytotoxic agent. An expressible form of a gene that can be used to induce selective cell death can be introduced into the recombinant cells. In such a system, cells expressing the protein encoded by the gene are susceptible to targeted killing under specific conditions or in, the presence or absence of specific agents. For example, an expressible form of a herpes virus thymidine kinase (herpes tk) gene can be introduced into the recombinant cells and used to induce selective cell death. When the introduced genetic material that includes the herpes tk gene is introduced into the individual, herpes tk will be produced. If it is desirable or necessary to kill the implanted recombinant cells, the drug ganciclovir can be administered to the individual which will cause the selective killing of any cell producing herpes tk. Thus, a system can be provided which allows for the selective destruction of implanted recombinant cells.

One skilled in the art would understand, based upon the disclosure provided herein, that the present invention encompasses production of recombinant cells to either provide CTHRC1 to or inhibit CTHRC1 expression in a mammal. That is, the cells can be used to administer CTHRC1 to an animal or to deliver a molecule (e.g., a knock-out targeting vector, an antisense nucleic acid, a ribozyme, and antibody that specifically binds with CTHRC1, and the like).

Administration of CTHRC1 to an animal can be used as a model system to study the mechanism of action of CTHRC1 or to develop model systems useful for the development of diagnostics and/or therapeutics for diseases, disorders or conditions associated with CTHRC1 expression.

Further, the delivery of CTHRC1 to an animal mediated by administration of recombinant cells expressing and secreting CTHRC1 can also be used to treat or alleviate a disease, disorder or condition where increasing the level of CTHRC1 mediates a therapeutic effect. More specifically, administration of CTHRC1 to an animal by administering a recombinant cell expressing a nucleic acid encoding CTHRC1 can be useful for treatment of fibrotic conditions among other things.

Alternatively, administration of recombinant cells comprising a nucleic acid, the expression of which inhibits or reduces CTHRC1 expression, activity, and/or secretion from a cell, can be used as a model for the development of diagnostics and/or therapeutics useful for diseases, disorders or conditions associated with or mediated by CTHRC1 expression, activity, and/or secretion. The present invention encompasses that the recombinant cells can produce the molecule that inhibits CTHRC1 expression thereby providing such molecule to the animal. Alternatively, without wishing to be bound by any particular theory, the recombinant cells themselves, which are otherwise functional cells, except for the inability to express CTHRC1, can perform the functions of otherwise identical but non-recombinant cells, without being subject to the CTHRC1 signaling pathway.

Cells, both obtained from an animal, from established cell lines that are commercially available or to be developed, or primary cells cultured in vitro, can be transfected using well known techniques readily available to those having ordinary skill in the art. Thus, the present invention is not limited to obtaining cells from a donor animal or from the patient animal itself. Rather, the invention includes using any cell that can be engineered using a nucleic acid of the invention such that the recombinant cell either expresses CTHRC1 (where it did not express CTHRC1 prior to being engineered, or where the cell produced CTHRC1 at a different level prior to the introduction of the nucleic acid into the cell) or the recombinant cell does not express CTHRC1 or expresses it at a lower level (where it expressed CTHRC1 before or expressed CTHRC1 at a different level prior to introduction of the nucleic acid into the cell).

Nucleic acids can be introduced into the cells using standard methods which are employed for introducing a gene construct into cells which express the protein encoded by the gene or which express a molecule that inhibits CTHRC1 expression. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA having a desired sequence into the cell. In some embodiments, recombinant retrovirus vectors are used to introduce DNA having a desired sequence into the cell. In some embodiments, standard calcium phosphate, DEAE dextran or lipid carrier mediated transfection techniques are employed to incorporate a desired DNA into dividing cells. Standard antibiotic resistance selection techniques can be used to identify and select transfected cells. In some embodiments, DNA is introduced directly into cells by microinjection. Similarly, well known electroporation or particle bombardment techniques can be used to introduce foreign DNA into cells. A second gene is usually co-transfected with and/or covalently linked to the nucleic acid encoding CTHRC1, or knock-out targeting vector or antisense molecule thereto. The second gene is frequently a selectable antibiotic-resistance gene. Transfected recombinant cells can be selected by growing the cells in an antibiotic that kills cells that do not take up the selectable gene. In most cases where the two genes are unlinked and co-transfected, the cells that survive the antibiotic treatment contain and express both genes.

Where an isolated CTHRC1 polypeptide, an antibody that specifically binds with CTHRC1, a CTHRC1 antisense nucleic acid, TGF-β1, soluble TGF-β1 receptor, and/or recombinant cells of the invention are administered to an animal either to increase or reduce the level of CTHRC1 present in the animal, one skilled in the art would understand, based upon the disclosure provided herein, that the amount of the polypeptide, nucleic acid, antibody, TGF-β1, soluble TGF-β1 receptor, or cell to be administered to the animal can be titrated by assessing the level of expression of CTHRC1 or the level of CTHRC1 polypeptide or nucleic acid encoding CTHRC1 present in the tissues of the animal.

Methods for assessing the level of CTHRC1 (e.g., using anti-CTHRC1 antibodies in Western blot or other immune-based analyses such as ELISA) and/or methods for assessing the level of CTHRC1 expression in a cell and/or tissues (e.g., using Northern blot analysis, RT-PCR analysis, in situ hybridization, and the like) are disclosed herein or are well known to those skilled in the art. Such assays can be used to determine the “effective amount” of CTHRC1 (whether using an isolated nucleic acid, antibody, antisense nucleic acid, ribozyme, recombinant cell, and the like) to be administered to the animal in order to reduce or increase the level of CTHRC1 to a desired level.

Methods Relating to CTHRC1 Regulation of Bone Morphogenetic Proteins

The invention includes a method of decreasing the level of BMP1 in a cell. This is because, as demonstrated by the data disclosed elsewhere herein, increasing the level of CTHRC1 in a cell decreases the level of BMP1. Thus, this surprising finding provides novel methods for affecting the level of BMP1 in a cell. More particularly, the data disclosed herein demonstrate, for the first time, that increasing CTHRC1 reduces the level of BMP1, while decreasing CTHRC1 mediates an increase in the level of BMP1 when compared with the level of BMP1 in an otherwise identical cell to which CTHRC1, or an inhibitor thereof (e.g., a CTHRC1 antisense nucleic acid) is not administered. Therefore, the present invention provides novel methods for increasing or decreasing the level of BMP1 by administering CTHRC1 or an inhibitor thereof, respectively. The skilled artisan, armed with the teachings provided herein, would readily appreciate when it would be desirable to increase or decrease the level of BMP I in a cell using the novel methods provided herein.

The invention also includes novel methods for increasing the level of a propeptide in a cell. This is because, as demonstrated by the data disclosed herein, administration of CTHRC1 to a cell mediates a surprising increase in the level of a propeptide in the cell when compared with an otherwise identical cell to which CTHRC1 is not administered. As disclosed elsewhere herein, the propeptide includes a procollagen and a propeptide of lysyl-oxidase, but other propeptides well known in the art, as appreciated by one skilled in the art once armed with the disclosure provided herein, are encompassed by the invention but not discussed further.

Further, the invention includes a method of inhibiting collagen formation by a cell. The method comprises contacting the cell with a BMP1 inhibiting amount of CTHRC1, thereby inhibiting collagen formation by the cell. This is because the data disclosed herein amply demonstrate that administration of CTHRC1 to a cell mediates a decrease in the level of BMP1 which thereby mediates, or is associated with, decreased collagen formation by the cell when compared with collagen formation by an otherwise identical cell to which CTHCR1 is not administered.

Likewise, the invention encompasses a method of decreasing bone matrix formation by a cell. The method comprises contacting a cell with a BMP1 inhibiting amount of CTHRC1, thereby decreasing bone matrix formation by the cell. This is because, as the data disclosed herein surprisingly demonstrate, administration of CTHRC1 to a cell mediates a decrease in the level of BMP1 in the cell, which is associated with, or mediates, a decrease in bone matrix formation by the cell when compared with bone matrix formation by an otherwise identical cell to which CTHRC1 is not administered. Thus, the skilled artisan would understand, based upon the disclosure provided herein, that the present invention encompasses methods of affecting bone matrix formation by a cell by administering CTHRC1 to the cell wherein CTHRC1 affects the level of BMP1, thereby affecting bone matrix formation.

Although the level of CTHRC1 administered to mediate a desired effect upon bone matrix formation is indicated elsewhere herein, the present invention is not limited in any way to this, or any other, amount of CTHRC1 to be administered. Rather, the skilled artisan would understand, based upon the disclosure provided herein, that the level of CTHRC1 to be administered can be readily established and adjusted to produce the desired level of decreased bone matrix production. Methods of assessing the level of bone matrix formation are well-known in the art, including methods disclosed elsewhere herein, and the present invention includes these, as well as methods to be developed in the future for assessing the level of bone matrix formation.

The invention encompasses a method of decreasing the level of collagen in a cell. The method comprises contacting a cell with a BMP1 inhibiting amount of CTHRC1, thereby decreasing the level of collagen in the cell. In one aspect, the collagen is type I collagen. This is because the data disclosed herein demonstrate, for the first time, that CTHRC1 mediates a decrease in the level of BMP1 in a cell, and further mediates a decrease in the level of collagen (e.g., type I collagen) in the cell. Thus, one skilled in the art would understand, based upon the disclosure provided herein, that the invention provides novel methods for affecting the level of collagen in a cell by either increasing or decreasing the level of CTHRC1 in the cell. Methods for increasing or decreasing the level of CTHRC1 in a cell are exemplified herein, and are also well-known in the art, and also encompass such methods as are developed in the future. Additionally, the amount of CTHRC1 to be administered to mediate the desired effect on the level of collagen in the cell is exemplified elsewhere herein, and can also be readily determined by the skilled artisan once armed with the teachings provided herein.

The invention also provides a method for increasing the level of procollagen in a cell. The method comprises contacting a cell with a BMP1 inhibiting amount of CTHRC1, thereby increasing the level of procollagen in the cell. That is, the data disclosed herein demonstrate that administration of CTHRC1 to a cell decreases the level of BMP1 in the cell and further mediates a decrease in the level of procollagen in the cell. Thus, by administering a level of CTHRC1 that mediates a detectable decrease in the level of BMP1 in the cell, the level of procollagen in the cell can be detectably reduced and such methods are encompassed by the invention.

Likewise, the invention includes a method of increasing the level of chordin in a cell. The method comprises contacting a cell with a BMP1 inhibiting amount of CTHRC1, thereby increasing the level of chordin in the cell. Ample support for this method is provided by the data disclosed herein demonstrating that administration of CTHRC1 to a cell mediates a decrease in the level of BMP1 in the cell and further mediates a detectable decrease in the level of chordin in the cell when compared with the level of chordin in an otherwise identical cell to which a BMP1 inhibiting amount of CTHRC1 is not administered. Thus, surprisingly, by decreasing the level of BMP1 in the cell, administration of CTHRC1 can be used to decrease the level of chordin and the invention encompasses such novel methods. The skilled artisan would appreciate, based upon the disclosure provided herein, when decrease of chordin would be desirable and the amount of CHTRC1 to mediate the desired decrease in chordin would also be easily determined by the skilled artisan using methods well known in the art.

The invention includes a method of inhibiting cross-linking of collagen fibrils in a cell. The method comprises contacting a cell with a BMP1 inhibiting amount of CTHRC1, wherein BMP1 is responsible for processing a propeptide lysyl-oxidase, and further wherein the lysyl-oxidase mediates cross-linking the collagen fibrils, thereby inhibiting cross-linking of collagen fibrils in the cell. This is because, as demonstrated by the data disclosed elsewhere herein, administration of CTHRC1 to a cell reduces the level of BMP1, thereby mediating a decrease in the processing of a propeptide (lysyl-oxidase) thereby inhibiting cross-linking of collagen fibrils. Thus, where the skilled artisan would desire inhibiting cross-liking of such fibrils, the present invention provides novel methods for doing so by administering CTHRC1. The surprising findings disclosed herein would be understood by the skilled artisan to provide novel methods for affecting cross-linking of collagen fibrils in a cell, and the amount of CTHRC1 to be administered, thus affecting the level of cross-linking of the fibrils, can be readily determined by one skilled in the art using methods disclosed elsewhere herein, as well as methods known in the art or to be developed in the future.

The invention also encompasses a method of inhibiting plaque rupture in a blood vessel. The method comprises administering a collagen matrix production enhancing amount of a CTHRC1 inhibitor to a blood vessel comprising a plaque, thereby inhibiting plaque rupture in the blood vessel. That is, the data disclosed elsewhere herein demonstrate that decreasing the level of CTHRC1 mediates an increase in the level of BMP1 and, because BMP1 activity can inhibit plaque rupture, decreasing the level of CTHRC1 can mediate decreased plaque ruptured which is desirable by those in the relevant art.

In one embodiment, the CTHRC1 inhibitor is selected from the group consisting of an antibody that specifically binds with CTHRC1 and a CTHRC1 antisense nucleic acid. However, the invention is not limited to these, or any other, inhibitor of CTHRC1. This is because one skilled in the art would readily appreciate, based upon the disclosure provided herein, that any inhibitor of CTHRC1, either exemplified herein, known in the art, or to be developed in the future, is encompassed by the invention to mediate a decrease in CTHRC1 thereby mediating a detectable increase in the level of BMP1.

The invention also includes a method of identifying a compound that affects collagen production in a cell. The method comprises contacting a cell comprising CTHRC1 with a test compound and assessing the level of CTHRC1 in the cell and comparing the level of CTHRC1 in an otherwise identical cell not contacted with the test compound. One skilled in the art, based upon the disclosure provided herein, would understand that a higher or lower level CTHRC1 in the cell contacted with the test compound compared with the level of CTHRC1 in the second otherwise identical cell not contacted with the test compound is an indication that the test compound inhibits collagen production in the cell. This is because, as demonstrated by the data disclosed herein, the level of CTHRC1 in a cell affects the level of collagen produced by the cell. thereby identifying a compound that inhibits collagen production in the cell.

In one aspect, the test compound is assayed for its ability to affect the level of BMP1. This is because, as demonstrated by the data disclosed herein, there is a surprising correlation between the level of CTHRC1 in a cell and the level of BMP1 in that cell. More specifically, there is an inverse correlation in that increased level of CTHRC1 mediates a decreased level of BMP1. Thus, a test compound that affects the level of CTHRC1 in a cell can be assayed for by its effect, if any, on BMP1 level. A test compound that affects, either increasing or decreasing the level of BMP1 in a cell, is a potential candidate compound that affects CTHRC1, as the skilled artisan would appreciate based upon the disclosure provided herein.

The invention includes a method of decreasing collagen formation in a patient in need thereof. The method comprises administering a BMP1 inhibiting amount of CTHRC1 to the patient, whereby inhibiting BMP1 reduces collagen production, thereby decreasing collagen formation in the patient. This is because, as demonstrated by the data disclosed herein, increasing CTHRC1 decreases the level of BMP1 in a cell, which, in turn, mediates a decrease in collagen formation. Thus, the skilled artisan would understand, once armed with the teachings provided herein, that the level of collagen formation can be inhibited by administering an amount of CTHRC1 that decreases the level of BMP1 in a cell, where the amount can be readily assessed according to the methods exemplified herein, methods known in the art, and/or methods to be developed in the future to determine the level of collagen formation, the level of CTHRC1 and/or the level of BMP1.

The method can be used wherein the patient has a condition mediated by collagen formation. Such condition includes, but is not limited to, wound scarring, wound healing, keloid formation, inflammation-associated scarring, pulmonary fibrosis, and angioplasty-associated vascular fibrosis.

The invention also includes a method of increasing bone matrix production in a cell. The method comprises administering an effective amount of an inhibitor of CTHRC1 to the cell, wherein inhibition of CTHRC1 increases the level of BMP1 in the cell, and further wherein increasing the level of BMP1 increases bone matrix production, thereby increasing bone matrix production. This is because, as amply demonstrated by the data disclosed herein, decreasing CTHRC1 mediates an increase in the level of BMP1 wherein the increased level of BMP1 mediates, or is associated with, increased bone matrix production.

The invention encompasses a method of increasing collagen production in a patient in need thereof. The method comprises administering an effective amount of an inhibitor of CTHRC1 to the patient, wherein inhibition of CTHRC1 increases the level of BMP1 in the patient, and further wherein increasing the level of BMP1 increases processing of fibrillar collagen, thereby increasing collagen production in the patient. This is because, as demonstrated elsewhere herein, decreasing CTHRC1 mediates an increase in the level of BMP1 wherein the increased level of BMP1 mediates, or is associated with, increased processing of fibrillar collagen, such that decreasing CTHRC1, by increasing processing by BMP1, increases collagen production in the patient. The skilled artisan would be, once armed with the teachings provided herein, be able to readily determine what patient is in need of such increased collagen production using the novel methods disclosed herein.

The invention encompasses a method of treating a disease mediated by expression of BMP1 in a patient in need thereof. The method comprises administering to the patient a BMP1 inhibiting amount of CTHRC1, thereby treating the disease mediated by expression of BMP1 in the patient. This is because the data disclosed herein demonstrate, for the first time, that administration of CTHRC1 decreases the level of BMP1 such that any condition mediated by BMP1 can be treated by administering CTHRC1, which, in turn, decreases the level of deleterious BMP1.

The invention includes a kit for decreasing the level of BMP1 in a cell. The kit comprises a BMP1 inhibiting amount of collagen triple helix repeat containing 1 (CTHRC1). The kit further comprises an applicator, and an instructional material for the use thereof. This is because, as demonstrated by the data disclosed herein, administration of CTHRC1 mediates a decrease in BMP1 in a cell. These surprising findings amply support a kit for decreasing BMP1 in a cell where the kit provides an amount sufficient to detectably decrease the level of BMP1 in a cell compared with the level of BMP1 in an otherwise identical cell to which CTHRC1 is not administered, and where the amount of CTHRC1 can be readily determined using assays exemplified herein, as well as those known in the art and/or to be developed in the future.

The skilled artisan would appreciate, based upon the disclosure provided herein, that the invention includes a kit for decreasing the level of BMP1 mRNA in a cell. The kit comprises a BMP1 inhibiting amount of collagen triple helix repeat containing 1 (CTHRC1), and an applicator, and an instructional material for the use of the kit. This is because, as pointed out elsewhere herein, the data disclosed herein demonstrate, for the first time, that administration of CTHRC1 mediates a decrease in BMP1 mRNA in a cell. These surprising findings amply support a kit for decreasing BMP1 mRNA in a cell where the kit provides an amount sufficient to detectably decrease the level of BMP1 mRNA in a cell compared with the level of BMP1 mRNA in an otherwise identical cell to which CTHRC1 is not administered, and where the amount of CTHRC1 can be readily determined using assays exemplified herein, as well as those known in the art and/or to be developed in the future.

Likewise, one skilled in the art would understand, once armed with the teachings provided herein, that the invention includes a kit for increasing the level of BMP1 in a cell. The kit comprises a BMP1 increasing amount of a collagen triple helix repeat containing 1 (CTHRC1) inhibitor, an applicator, and an instructional material for the use of the kit. This is because the data disclosed herein demonstrate, surprisingly, that inhibiting CTHRC1 in a cell mediates an increase in the level of BMP1. Thus, the skilled artisan would appreciate, based upon the disclosure provided herein, that the invention encompasses increasing the level of BMP1 by decreasing the level of CTHRC1, and a kit for doing so using, among other things, methods known in the art for decreasing the level of a protein in a cell.

Similarly, the invention includes a kit for increasing the level of a propeptide in a cell. The kit comprises a BMP1 inhibiting amount of CTHRC1, an applicator, and an instructional material for the use thereof. This is because the data disclosed herein surprisingly demonstrate that administering CTHRC1 decreases the level of BMP1 and, thereby, increases the level of a propeptide (e.g., procollagen, a propeptide of lysyl-oxidase, and the like) in a cell.

The invention also includes a kit for inhibiting collagen formation by a cell. The kit comprises a BMP1 inhibiting amount of CTHRC1, an applicator, and an instructional material for the use thereof. This is because the data provided herein demonstrate that administration of CTRHC1 decreases the level of BMP1 thereby inhibiting collagen formation.

The invention encompasses a kit for decreasing bone matrix formation by a cell where the kit comprises a BMP1 inhibiting amount of CTHRC1, an applicator, and an instructional material for the use of the kit. This is because, as discussed previously elsewhere herein, the data disclosed herein demonstrate that administration of CTHRC1 mediates a decrease in the level of BMP1 such that bone matrix formation is decreased.

One skilled in the art, based upon the disclosure provided herein, would understand that the invention encompasses a kit for decreasing the level of collagen in a cell. The kit comprises a BMP1 inhibiting amount of CTHRC1, an applicator, and an instructional material for the use thereof. This is because, as discussed previously elsewhere herein, the data disclosed herein demonstrate that administration of CTHRC1 decreases the level of BMP1 such that the level of collagen (e.g., type I collagen) in a cell is also decreased.

The skilled artisan would appreciated, based upon the disclosure provided herein, that the invention encompasses a method of increasing the level of bone morphogenetic protein 4 (BMP4) in a cell. The method comprises contacting a cell expressing BMP4 with CTHRC1 in an amount sufficient to increase the level of the BMP4 in the cell when compared with the level of BMP4 in an otherwise identical cell not contacted with CTHRC1, or the level of BMP4 in the cell prior to the cell being contacted with CTHRC1. This is because the data disclosed elsewhere herein demonstrate that contacting a cell with CTHRC1, or otherwise administering CTHRC1 (e.g., a nucleic acid encoding CTHRC1 and/or a CTHRC1 polypeptide, or fragment thereof), increases the level of BMP4 in the cell.

Likewise, the invention includes a method of increasing the level of BMP4 promoter activity in a cell. The method comprises contacting a cell with CTHRC1 in an amount sufficient to increase the level of BMP4 promoter activity in the cell. This is because the data disclosed elsewhere herein demonstrate, for the first time, that administration of CTHRC1 mediates an increase in the level of BMP4 promoter activity.

The skilled artisan would also appreciate, based upon the disclosure provided herein, that the invention encompasses a method of promoting bone growth in a mammal. The method comprises administering CTHRC1 to the mammal in an amount sufficient to increase the level of BMP4, where the data demonstrate that increasing the level of BMP4 detectably increases the level of bone growth when compared with the level of bone growth where CTHRC1 is not administered to an otherwise identical mammal, or when compared with bone growth in the mammal prior to administration of CTHRC1. The skilled artisan, once armed with the teachings provided herein, can readily determine the route and dose of administration as well-known in the art in that the level of BMP4 in the pertinent bone forming cells can be readily assessed by a plethora of methods known in the art to determine the efficacy of administration of CTHRC1.

The invention includes a method of promoting differentiation of a stem cell. The method comprises contacting a stem cell with CTHRC1 in an amount sufficient to increase the level of BMP4 in the stem cell, thereby promoting differentiation of the stem cell. This is because the data disclosed herein demonstrate that administration of CTHRC1 mediates an increase in the level of BMP4 in a cell compared with a cell to which CTHRC1 is not administered, and where it is known that a biological activity of BMP4 is to promote the differentiation of a stem cell. Thus, one skilled in the art, armed with the teachings provided herein, would understand that administering CTHRC1 to a stem cell, where CTHRC1 mediates an increase in BMP4 in the cell, promotes differentiation of the stem cell.

One skilled in the art would understand, based upon the disclosure provided herein, that the invention encompasses a method of decreasing the level of osteopontin (OPN) in a cell. The method comprises contacting a cell with CTHRC1 in an amount sufficient to decrease the level of OPN, thereby decreasing the level of OPN in the cell. This is because the data disclosed herein demonstrate that contacting, or otherwise administering, CTHRC1 to a cell mediates a decrease in OPN level in that cell compared with the level of OPN in an otherwise identical cell not contacted with CTHRC1.

Conversely, the skilled artisan, based upon the disclosure provided herein, would also appreciate that the invention includes a method of increasing the level of OPN in a cell. The method comprises contacting a cell with a CTHRC1 inhibiting amount of a CTHRC1 inhibitor, thereby increasing the level of OPN in the cell. This is because the data disclosed herein demonstrate that decreasing CTHRC1 in a cell mediates an increase in the level of OPN in the cell when compared with the level of OPN in an otherwise identical cell not contacted with an inhibitor of CTHRC1, which inhibitor mediates a decrease in the amount and/or activity of CTHRC1 in the cell.

The invention includes a method of identifying a compound that effects a CTHRC1 -mediated reduction of BMP4 in a cell. The method comprises contacting a CTHRC1-containing cell with a test compound, wherein a lower level of BMP4 in the cell contacted with the test compound compared with the level of BMP4 in a second otherwise identical cell not contacted with the test compound is an indication that the test compound reduces the level of BMP4 in the cell, and further wherein the test compound affects the activity of CTHRC1, thereby identifying a compound that effects a CTHRC1 -mediated reduction of BMP4 in the cell. This is because a compound that affects the BMP4 level in a cell is a potential candidate compound for affecting the level (e.g., the amount, activity, or both) of CTHRC1 in a cell. This is because the data disclosed herein demonstrate, surprisingly, that the level of CTHRC1 in a cell affects the level of BMP4 in a cell, such that increasing CTHRC1 increases BMP4 and decreasing CTHRC1 decreases the level of BMP4 in the cell. Based on this surprising correlation, a test compound that affects BMP4 in a cell is a potential candidate compound for affecting the level of CTHRC1 in a cell.

One skilled in the art would appreciate, based upon the disclosure provided herein, that the invention encompasses a method of treating a disease mediated by BMP4 in a human in need thereof. The method comprises administering to a human afflicted with a disease mediated by BMP4 a CTHR1 inhibiting amount of a CTHRC1 inhibitor, thereby treating the disease mediated by BMP4 in the human in need thereof. This is because, as discussed previously elsewhere herein, the data disclosed herein demonstrate that inhibiting CTHRC1 mediates a decrease in BMP4 such that, where a disease is mediated by BMP4, inhibiting CTHRC1 provides an effective method for inhibiting BMP4 and, thereby, treating the disease.

The invention also includes a method of treating a disease mediated by under-expression of BMP4 in a human in need thereof. The method comprises administering to a human afflicted with the disease a BMP4 expression-inducing amount of CTHRC1. This is because the data disclosed herein demonstrate that increasing the level of CTHRC1 mediates in increase in the level of BMP4 compared with the level of BMP4 where CTHRC1 is not administered. Thus, where an increase in the level of BMP4 would provide a treatment, administration of CTHRC1 can provide a beneficial effect. The skilled artisan, armed with the teachings of the present invention, can readily determine a disease that can be treated by increasing the level of BMP4, since methods for identifying such disease, and patients afflicted therewith, are known in the art.

The skilled artisan, based upon the disclosure provided herein, would understand that the invention includes a method of increasing the level of a muscle segment homeobox 1 (Msx1) in a cell. The method comprises contacting a cell expressing BMP4 with a collagen triple helix repeat containing 1 (CTHRC1) in an amount sufficient to increase the level of the BMP4 in the cell. This because increasing the level of BMP4 mediates an increase in the level of Msx1, and because the data disclosed elsewhere herein demonstrate, for the first time, that contacting a cell with CTHRC1 increases the level of BMP4 in the cell compared with the level of BMP4 in an otherwise identical cell not contacted with CTHRC1. The data also show that contacting a cell with CTHRC1 mediates a detectable increase in the level of Msx1 in the cell, thereby demonstrating that administration of CTHRC1 increases the level of Msx1 in the cell.

The invention includes a kit for increasing the level of bone morphogenetic protein 4 (BMP4) in a cell, where the kit comprises an amount of CTHRC1 sufficient to increase the level of BMP4 in a cell, an applicator, and an instructional material for the use of the kit. This is because, as discussed previously elsewhere herein, the data disclosed herein demonstrate that administering CTHRC1 to a cell mediates a detectable increase in the level of BMP4 in the cell compared with the level of BMP4 in an otherwise identical cell to which CTRHC1 is not administered.

C. Methods of Diagnosis and Assessment of Therapies

The present invention includes methods of diagnosis certain diseases, disorders, or conditions such as, but not limited to, constrictive remodeling, arterial restenosis, adventitial fibrosis, excessive wound healing responses, scarring, keloids, excessive bone formation, fracture healing, ectopic ossification (malignant and benign), fibrosis in any organ or tissue (e.g., liver fibrosis and lung fibrosis), altered bone density, altered bone growth, collagen diseases, and the like, which are associated with or mediated by abnormal expression of CTHRC1.

The invention includes a method of diagnosing tissue damage, constrictive remodeling, arterial restenosis, adventitial fibrosis, excessive wound healing responses, scarring, keloids, excessive bone formation, fracture healing, ectopic ossification (malignant and benign), fibrosis in any organ or tissue (e.g., liver fibrosis and lung fibrosis), altered bone density, altered bone growth, premature calcification, collagen-related diseases, and the like, in a patient mammal. This is because, as demonstrated by the data disclosed herein, there is a correlation between altered expression of CTHRC1, when compared to expression of CTHRC1 in otherwise identical but undamaged, normal tissue, and tissue injury, constrictive remodeling, arterial restenosis, adventitial fibrosis, excessive wound healing responses, scarring, keloids, excessive bone formation, fracture healing, ectopic ossification (malignant and benign), fibrosis in any organ or tissue (e.g., liver fibrosis and lung fibrosis), altered bone density, altered bone growth, premature calcification, collagen diseases, and the like, such that assessing the level of CTHRC1 expression is a useful diagnostic for these diseases, disorders, or conditions associated with altered expression of CTHRC1.

The method comprises obtaining a biological sample from the mammal and comparing the level of CTHRC1 (expression, amount, activity) in the sample with the level of CTHRC1 in a sample from a normal person who is not afflicted with tissue damage, ectopic ossification, and organ fibrosis. A higher level of CTHRC1 in the sample from the patient compared with the level of CTHRC1 in the sample obtained from a person not afflicted with tissue damage, ectopic ossification, and organ fibrosis an indication that the patient is afflicted with tissue damage, ectopic ossification, and organ fibrosis. This is because, as disclosed elsewhere herein, an increased level of CTHRC1 expression is associated with tissue damage, ectopic ossification, organ fibrosis, bone mineralization, skin wounding, bone density and/or bone growth, lack of dorsal closure, spina bifida-like phenotype, collagen-related phenotypes, and vascular injury.

In one aspect, the biological sample is selected from the group consisting of a lung biopsy, an aorta sample, a smooth muscle cell (SMC) sample, an endarterectomy sample, a liver biopsy, any biopsy from a wound, and the like.

The invention includes a method of assessing the effectiveness of a treatment for arterial restenosis in a mammal. The method comprises assessing the level of CTHRC1 expression, amount, and/or activity, before, during and after a specified course of treatment for arterial stenosis since arterial restenosis and/or arterial fibrosis is associated with increased CTHRC1 expression. This is because, as stated previously elsewhere and demonstrated by the data disclosed herein, CTHRC1 expression, amount and/or activity is associated with or mediates decreased increased cell proliferation which is feature of certain disease states (e.g., constrictive remodeling, adventitial fibrosis and arterial restenosis), and is also associated with, or mediates, increased collagen matrix production. Thus, assessing the effect of a course of treatment upon CTHRC1 expression/amount/activity indicates the efficacy of the treatment such that a lower level of CTHRC1 expression, amount, or activity indicates that the treatment method is successful.

The data disclosed herein should allow the identification and characterization of the CTHRC1 ligand/receptor. This is useful since antagonism of the CTHRC1 ligand, receptor, or both, should provide useful in treatment of diseases, disorders or conditions mediated by CTHRC1 ligand/receptor signaling such as, but not limited to, arterial restenosis, constrictive remodeling, adventitial fibrosis, fibrosis in any organ or tissue (e.g., liver, lung, among others), hypertrophic scar tissue (i.e., keloids), excessive bone formation, ectopic ossification (malignant and benign), and the like.

IX. Kits

The invention includes various kits which comprise a compound, such as a nucleic acid encoding CTHRC1, an antibody that specifically binds CTHRC1, a nucleic acid complementary to a nucleic acid encoding CTHRC1 but in an antisense orientation with respect to transcription, and/or compositions of the invention, an applicator, and instructional materials which describe use of the compound to perform the methods of the invention. Although exemplary kits are described below, the contents of other useful kits will be apparent to the skilled artisan in light of the present disclosure. Each of these kits is included within the invention.

In one aspect, the invention includes a kit for alleviating a disease mediated by altered expression of CTHRC1. The kit is used pursuant to the methods disclosed in the invention. Briefly, the kit may be used to contact a cell with a nucleic acid complementary to a nucleic acid encoding CTHRC1 where the nucleic acid is in an antisense orientation with respect to transcription to reduce expression of CTHRC1, or with an antibody that specifically binds with CTHRC1, wherein the decreased expression, amount, or activity of CTHRC1 mediates an beneficial effect.

The kit further comprises an applicator useful for introducing the nucleic acid or antibody protein of the invention into the cell. The particular applicator included in the kit will depend on, e.g., the recombinant DNA method used to introduce the nucleic acid into the cell and such applicators are well-known in the art and may include, among other things, a pipette, a syringe, a dropper, and the like. Moreover, the kit comprises an instructional material for the use of the kit. These instructions simply embody the disclosure provided herein.

The kit includes a pharmaceutically-acceptable carrier. The composition is provided in an appropriate amount as set forth elsewhere herein. Further, the route of administration and the frequency of administration are as previously set forth elsewhere herein.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### CTHRC1 : Novel Modulator of Collagen Matrix

The experiments presented in this example may be summarized as follows.

In order to identify novel factors involved in mediating arterial remodeling in response to injury, suppressive subtractive hybridization was performed using mRNA from normal and balloon-injured rat arteries. A novel nucleic acid sequence was identified using this approach and a full length 1235 bp cDNA clone was isolated by screening a cDNA library prepared from 8 day balloon-injured rat carotid arteries and aortae. This cDNA clone comprises an open reading frame (ORF) of about 245 amino acids having no significant homology to any known protein. This protein, referred to herein as CTHRC1, was previously termed REMODELIN, REMODEL, and more previously termed AIBE for Adventitia Inducible and Bone Expressed protein. CTHRC1 comprises, inter alia, a potential transmembrane domain and five potential N-myristoylation sites which can target the molecule to the cell membrane.

In situ hybridization analysis disclosed that CTHRC1 mRNA expression is remarkably restricted to the adventitia of balloon-injured vessels with maximal expression detected at 8 days after carotid artery balloon denudation and with no detectable expression in normal arteries. CTHRC1 expression in the adventitia was no longer detectable at 6 weeks after balloon injury.

The data disclosed herein further demonstrate that during mouse embryogenesis, CTHRC1 expression was prominent in developing bone starting at about 12 days post coitus (dpc), while Northern blot analysis demonstrated that only low levels of CTHRC1 expression were detected in brain and lung of the adult animal.

Antibodies specific for CTHRC1 were generated and used to characterize expression of the protein by in vitro translation, in various cell lines which endogenously express CTHRC1, injured arteries, and cells transfected with a CTHRC1 expression vector.

CTHRC1 was shown to alter the expression of several cellular genes including TGF-β1, collagen III, biglycan, osteopontin, ALP, and BMP-4. Further, CTHRC1 was shown to inhibit Cbfal-mediated activation of the osteocalcin promoter, as determined using a luciferase assay.

Injection of CTHRC1 mRNA into frog embryos caused severe developmental abnormalities, including, but not limited to, inhibition of FGF-induced mesoderm formation, failure of neural tissue cells to migrate, and failure of dorsal closure, abnormal head development, and formation of a split tail.

MC3T3-E1 cells transfected with CTHRC1 antisense exhibited elongated, fibroblastic morphology, and exhibited increased cell turnover, suggesting that CTHRC1 is involved in cell-matrix and cell-cell interaction(s).

Analysis of CTHRC1 transgenic mice revealed severe phenotypes including hemorrhaging, dwarfism, skeletal abnormalities including decreased bone density, and severe myopathy.

The Results of the experiments presented in this example are now described.

Identification of Injury Inducible Factor

Suppressive subtractive hybridization was performed between cDNA expressed in normal rat carotid artery/aorta and cDNA expressed in 8 day balloon injured carotid/aorta using the PCR-Select kit from Clontech Laboratories, Inc. (Palo Alto, Calif.), to identify genes that are involved in the arterial remodeling response to injury. The normal vessel provided the “driver” cDNA and the injured vessel provided the “tester” cDNA.

Partial sequences of approximately 300 clones were obtained by automated sequencing and the sequence identities were determined by searching GenBank databases, including non-redundant and EST (expressed sequence tag) databases which are publicly available at http://www.ncbi.nlm.nih.gov/blast/blast.cgi. Those sequences not matching a known gene (usually corresponding murine or human ESTs) were identified in the database and were pursued further.

Duplicate slot blots containing the series of cDNA clones were hybridized with ³²P-dCTP labeled cDNA prepared from either normal vessel RNA or from balloon-injured vessel RNA. The clones that exhibited increased expression in the injured vessels were then further tested using Northern blot technique with RNA from both normal and balloon-injured rat arteries.

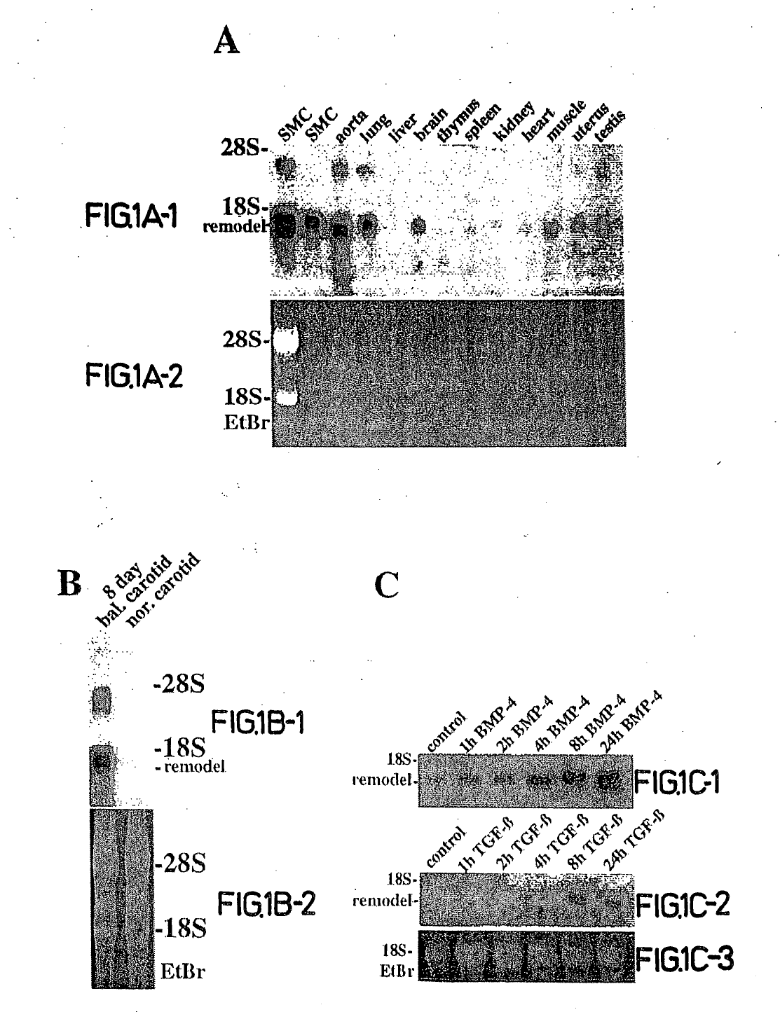

The data disclosed herein demonstrate that CTHRC1 expression was essentially not detectable in normal vessels while injured vessels exhibited a dominant 1.2 kb transcript (FIG. 1B-1). The sequences exhibiting detectable increased expression in the injured vessels compared with normal vessels were further examined for expression in various organs using Northern blot. In order to select for genes that might be specific for the vasculature, those clones that were predominantly expressed in vascular tissues like lung and brain, in addition to showing expression in the aorta or carotid artery, were pursued. The data disclosed herein demonstrate that CTHRC1 showed low levels of expression in lung and brain (FIG. 1A-1).

Clones identified using the above-described screening approach were then used to make ³⁵S-UTP labeled sense and antisense strand which were, in turn, used for in situ hybridization on normal and balloon-injured rat carotid artery sections (4, 8, 14, and 28 days after injury), as well as on sections from staged mouse embryos.

To determine expression in quiescent versus proliferating/migrating endothelium, en face preparations of 7 and 14 day injured aortas were also used in the in situ hybridization study as described in Lindner and Reidy (1993, Circ. Res. 73:589-595). In the injured aortae, endothelial regeneration occurs from the intercostal arteries giving rise to migrating/proliferating endothelial cells at the wound edge as well as quiescent endothelium in the monolayer away from the wound edge.

Among the clones exhibiting modulated expression in response to injury, CTHRC1 was expressed in the adventitia of injured vessels but was absent from normal adventitia (FIGS. 2A and 2B). Strong expression of CTHRC1 was detected at 8 days after injury with less expression at 14 days (FIGS. 2B and 2C). Surprisingly, there was no appreciable CTHRC1 expression detected in the media and in the developing neointima despite the fact that Smooth Muscle Cells (SMC) in vitro expressed the 1.2 kb transcript (FIGS. 2B and 2C). No other gene is known in the art which is specifically induced in the adventitia and is not detectably induced in the neointima. Longer exposure or loading of larger amounts of RNA from in vitro SMC also revealed a less abundant transcript of about 3.5 kb (FIG. 1A-1).

At 4 weeks after balloon injury, CTHRC1 expression was nearly undetectable in the adventitia. At the time when CTHRC1 is expressed, the adventitia shows rapid proliferation of myofibroblasts as well as a subsequent sharp decline in cell number that is accompanied by abundant synthesis of collagens type I and type III (Smith et al., 1999, Cir. Res. 84:1212-1222). Interestingly, in the staged mouse embryos, CTHRC1 expression was detected in the mesoderm at 11.5 days post coitus (dpc) (FIG. 2E), which expression later became restricted to the developing bone (FIG. 2F). Lower levels of CTHRC1 expression were also detected in the cortical bone of a femur from a rat pup. During development expression of CTHRC1 was prominent in developing bone such as the skull (FIGS. 2F, 2G and 2H). CTHRC1 continued to be expressed in osteoblasts adjacent to mineralized bone (FIGS. 21 and 2J).

In addition, full thickness skin incisions undergoing wound healing and remodeling revealed strong expression along the incision (FIG. 2L) while no expression was detectable in normal skin (FIG. 2K).

A full length CTHRC1 clone was obtained using a size-fractionated (500 bp cut-off) cDNA library prepared using mRNA extracted from 8 day balloon-injured rat aortas and carotid arteries using a Lambda Zap Express system (Stratagene, La Jolla, Calif.). After excision with a helper phage, the isolated cDNA clones were ligated into the pBK-CMV vector (Stratagene, La Jolla, Calif.), which allows for convenient expression in mammalian cells using the CMV promoter. This library is expected to contain sequences expressed in proliferating SMC, endothelial cells (EC), and fibroblasts as well as their quiescent counterparts. In addition, sequences from inflammatory cells, predominantly macrophages, are also expected to be present in the library.

The 230 bp CTHRC1 clone obtained using the differential screen approach was then used to probe the library and six clones were isolated and sequenced. Five clones started within 50 bp upstream of a putative translational AUG start site at nucleotide position 116 (FIG. 4Ai). The longest clone contained an additional 60 bp of 5′ sequence which contained a potential additional in-frame AUG translational start site at position 19 (FIG. 4Ai). The 230 bp clone was designated CTHRC1-short (CTHRC1_{S}) and the 290 bp CTHRC1 clone was designated CTHRC1-long (CTHRC1_{L}).

Searching The Institute for Genomic Research (TIGR) sequence database for the human homolog of CTHRC1, identified a 771 bp of 3′ sequence. Using RNA from cultured human aortic SMC, 5′ RACE (rapid amplification of cDNA ends) cloning was performed to identify the missing approximately 500 bp sequence located at the 5′ end. The sequence alignment of rat (SEQ ID NO:2) and human CTHRC1 (SEQ ID NO:4) is shown in FIG. 4. Interestingly, the human sequence did not have the additional 5′ AUG translational start site but only contained the AUG codon at position 114. A 5′ primer located upstream of this AUG start site and a 3′ primer were designed to verify that the AUG start site at position 19 of the rat sequence was not a cloning artifact. Reverse transcription polymerase chain reaction (RT-PCR) analysis and sequencing was performed to confirm the presence of the AUG-19 codon. The overall identity between the human and rat CTHRC1 sequence was 78.3% at the nucleotide level using a blast 2 algorithm search as provided at the web site http://www.ncbi.nlm.nih.gov/gorf/b12.html, which web site is publicly available. Further, homology between the rat (SEQ ID NO:2) and human CTHRC1 (SEQ ID NO:4) amino acid sequences is greater than about 97% using the blast 2 algorithm search strategy described previously.

Amino Acid Sequence and Domains within CTHRC1

In vitro translation was performed using a kit (Promega Corp., Madison, Wis.) comprising rabbit reticulocyte lysate and ³⁵S-methionine. The data disclosed herein demonstrate that CTHRC1_{L} contained two putative transcriptional start sites compatible with the Kozak rule (i.e., at positions 19 and 116) while CTHRC1 s had only the AUG₁₁₆ site. In vitro translation revealed that the CTHRC1_{L} construct expressed preferentially the long form but, to a lesser extent, the short form of CTHRC1 was also expressed from the CTHRC1_{L} construct (FIG. 5A). However, the CTHRC1_{S} construct expressed only the shorter form. The apparent molecular weights of the long and short form were approximately 34 kDa and 30 kDa, respectively (FIG. 5A). Since the human sequence does not have the additional AUG codon at the 5′ end, it is most likely that in vivo translation starts at AUG₁₁₆. This is predicted to result in a 245 amino acid (aa) rat protein while the human homolog has about 243 amino acids (SEQ ID NO:4) due to a 2 amino acid deletion in the amino terminus.

A leucine-rich hydrophobic region is located near the amino terminus and, without wishing to be bound by any particular theory, this region is predicted to be a cleavable signal peptide (from about amino acid residue number 1 to amino acid residue 32), which would result in a 213 aa mature peptide having a molecular weight of about 23.1 kDa and a theoretical pI of 6.57 for the human CTHRC1. However, if the aa1-aa32 peptide is not a signal sequence and does not get cleaved, it is predicted to be a definitive transmembrane domain. The rat CTHRC1_{L} protein would result in a 277 aa protein which lacks a predicted signal sequence (SEQ ID NO:2).

At the amino acid level, human and rat CTHRC1 were 95% identical (FIG. 4B). The carboxy terminal half of the molecule was more highly conserved with over 99% identity between the two species. Remarkably, the amino acid composition contains 4.7% cysteine, 2.4% tyrosine, and 2% tryptophan residues. A glycine-rich domain also found in many collagens is located between amino acid residue 59 and amino acid residue 93. A putative N-glycosylation site is located at about amino acid residue 188 to about amino acid residue 191, a putative protein kinase C (PKC) phosphorylation site at amino acid residue 146 to amino acid residue 148, and four casein kinase II (CD2) phosphorylation sites. Furthermore, there are 5 N-myristoylation sites located throughout the molecule. A summary of these putative motifs is depicted in FIG. 3.

Functional Characterization of CTHRC1

Contrary to SMC in the vessel wall, cultured rat aortic SMC growing in DMEM supplemented with 10% bovine serum expressed CTHRC1 mRNA. Expression was inducible by TGF-β in 3T3 cells as well as SMC with peak expression detected after 8 hours of stimulation. Expression levels were still elevated after 24 hours and no induction followed stimulation of cells with FGF-2.

Since CTHRC1 is also expressed in bone, expression in MC3T3 cells, a bone derived cell line, was also examined. The response to TGF-β stimulation was similar to SMC and NIH3T3 cells with maximal induction occurring after 8 hours (FIG. 1C). Stimulation with bone morphogenetic protein-4 (BMP-4) caused a similar induction of CTHRC1 mRNA in MC3T3 cells as TGF-β (FIG. 1C).

Antibodies that Specifically Bind CTHRC1

The peptide corresponding to the carboxy terminal 15 amino acids of CTHRC1 was coupled to keyhole limpet hemocyanin (KLH) and used to immunize two rabbits. One of the rabbits produced antiserum that had a titer of greater than about 1:64,000. The antiserum (used at a 1:5000 dilution) detected a specific band with a molecular weight of approximately 30 kDa in lysates prepared from 10 day balloon-injured rat carotid arteries while this band was absent in lysates prepared from normal vessels (FIG. 5C). The same specific band was also detected in lysates from cultured SMC.

In addition, SMC cultured in vitro also expressed a slightly larger immunoreactive band of approximately 34 kDa. However, concentrated conditioned medium obtained from SMC contained no detectable immunoreactivity, indicating that if any CTHRC1 is secreted, it is at low levels beyond the level of detection. Further, the detectable bands were specific since the preimmune IgG from the same rabbit did not react with these protein bands (FIG. 5C).

Expression of GFP-tagged CTHRC1 in Transfected Cells

CTHRC1 cDNA was cloned into a green fluorescent protein (GFP)- and hemagglutinin (HA)-tagged expression vector to produce CTHRC1/tag polypeptide fusion proteins. The localization of the GFP-CTHRC1 fusion protein was then assessed with regard to its cellular localization following transfection of NIH3T3 and 293 cells with the construct encoding the fusion protein. The data disclosed herein demonstrate that fluorescence was distributed homogeneously throughout the cell with absence of GFP in the nucleus. This staining pattern is compatible with cytosolic and/or cell membrane localization. The transfection efficiency obtained using the GFP-CTHRC1 construct was consistently lower than with the GFP control vector.

Expression of myc-Tagged CTHRC1 in Transfected Cells

The coding region of rat CTHRC1 was cloned into a mammalian expression vector comprising a myc tag at the carboxy terminus (pcDNA3.1 myc/his, Invitrogen, Carlsbad, Calif.). The sequence of the coding region used in the construct is depicted in FIG. 10 (SEQ ID NO:9).

Transient transfections were performed using bovine aortic endothelial cells (BAE) and NIH3T3 cells. The cell lines were analyzed for transgene expression 24 hours and 48 hours after transfection using Western blotting and immunostaining using an anti-myc monoclonal antibody (Zymed, South San Francisco, Calif.).

Interestingly, the data disclosed herein demonstrate that very little expression remained at 48 hours post-transfection (FIG. 5F). Without wishing to be bound by any particular theory, these data suggest that the transfected cells were lost from the culture. In comparison, the same cell line transfected in parallel with an unrelated cDNA (EP1) using the same vector exhibited significantly higher levels of expression at 48 hours than at 24 hours (FIG. 5F).

The possibility that overexpression of CTHRC1 results in cell death was examined further using immunohistochemistry, confocal microscopy and flow cytometry for cell cycle analysis. BAE cells transiently transfected with myc-tagged CTHRC1 exhibited a 15% increase in accumulation of cells in G0-G1 at 24 hours post-transfection (transfection efficiency approximately 15%). Western blotting of these transfectants using the anti-myc antibody demonstrated that by 48 hours post-transfection very little immunoreactivity remained, indicating that the transfected cells were lost from the culture.

Confocal microscopy of CTHRC1 transfected NIH3T3 cells demonstrated localization of the myc-tagged protein in very small vesicles distributed throughout the cytoplasm (FIG. 5B).

Expression of CTHRC1 in Various Cell Types

Vessel wall lysates were prepared from rat carotid arteries harvested at 1, 4, 7, 14, and 28 days after balloon injury. These lysates (30 micrograms of protein in each lane) were analyzed by immunoblotting using an antibody raised against the carboxyterminal peptide of CTHRC1. The highest levels of CTHRC1 were seen at 4 and 7 days after injury with a decline to near control levels at 28 days post injury (FIG. 5C).

Smooth muscle cells in vitro expressed CTHRC1 mRNA while this cell type showed very little expression in vivo. Therefore expression of CTHRC1 was examined using a variety of different cell lines using immunoblotting analysis (FIG. 5D). The cell lines included NIH3T3, bovine aortic (BAE), PAC-1 (a rat smooth muscle cell line), A7r5 (a rat smooth muscle cell line), RASMC (rat aortic SMC primary culture), 293, BASMC (bovine aortic SMC), 10T/½, human umbilical vein endothelial cells (HUVEC), A43 1, and human aortic SMC (HASMC). The data disclosed herein demonstrate detection of a prominent immunoreactive band with an apparent molecular weight of about 34 k Da that was present in all cell lines tested. These data indicate that the antibody reacted with mouse, rat, bovine, and human homologs of CTHRC1 (FIG. 5D). A less abundant protein band having an apparent molecular weight of about 30 kDa was present in some cell lines and some additional larger immunoreactive bands were also detected. Without wishing to be bound by any particular theory, the 30 kDa band may reflect differences in glycosylation or phosphorylation of CTHRC1.

Regulation of CTHRC1 Expression

The regulation of CTHRC1 expression by TGF-β was further investigated using MC3T3 cells, a bone derived cell line. Immunoblotting of cell lysates harvested after 24, 48, and 72 hours after stimulation with TGF-β demonstrated increased expression levels of CTHRC1 protein compared to controls (FIG. 5E).

The data disclosed herein demonstrate that blocking signaling via the TGF-β receptor type II by the addition of a soluble TGF-β receptor type II (Biogen, Cambridge, Mass.) to the cells, inhibited expression of CTHRC1 protein (FIG. 5E). MC3T3 cells were treated with 1 ng/ml of TGF-β1 (TGF-β) or 100 ng/mi soluble TGF-β receptor type II (sol. TGF-βRII), the cells were harvested at various time points, and 30 micrograms of protein were applied to each polyacrylamide gel lane (FIG. 5E). The data disclosed herein demonstrate that TGF-β1 stimulated CTHRC1 expression while inhibition of TGF-β signaling inhibited CTHRC1 expression.

Effects of CTHRC1 on Gene Expression

Stable NIH3T3 and MC3T3 cell lines overexpressing CTHRC1 under the control of the CMV promoter were established. To identify genes whose expression might be changed in response to altered levels of CTHRC1, Northern blots from CTHRC1 overexpressing and antisense CTHRC1 transfected cell lines were performed (FIG. 6). CTHRC1 overexpressing NIH3T3 cells expressed considerable lower levels of TGF-β1, collagen III, and biglycan while the mRNA levels of the same genes were elevated in the antisense transfected cells. Most dramatic was the >10-fold upregulation of osteopontin and ALP in the antisense transfected cells. The dramatic effects of CTHRC1 on osteopontin expression were further analyzed using a luciferase assay. NIH3T3 cells were cotransfected with a construct in which the osteopontin promoter driving luciferase activity (provided by Dr. Liaw) and the CTHRC1 expression construct under the control of the CMV promoter. As shown in FIG. 6, NIH3T3 cells express endogenous osteopontin. Compared to vector transfected cells, CTHRC1 decreased luciferase activity by 80% (FIG. 7). As discussed herein, CTHRC1 affects bone formation, and thus, it was of interest to determine the effect of CTHRC1 on Cbfal-dependent activity of the osteocalcin promoter. NIH3T3 and MC3T3-E1 cells were cotransfected with an osteocalcin-luciferase construct (containing OSE2 and others, provided by Dr. Karsenty), Cbfal, and CTHRC1 (or empty vector). CTHRC1 completely inhibited the CbfaI-mediated increase in osteocalcin-driven luciferase activity (FIG. 7).

Bacterial Expression of CTHRC1

CTHRC1 protein with a 6x histidine tag was expressed in E. coli, and the recombinant protein was characterized by immunoblotting. Under non-reducing conditions, bands of 74, 49, and 25 kDa reacted with anti-his tag antibody (FIG. 8, lane 1). Under reducing conditions, his-tagged recombinant CTHRC1 protein runs as a single band of approximately 28 kDa (FIG. 8, lanes 2 and 3) which is similar in size to the native protein detected on immunoblots (FIG. 5A). Without wishing to be bound to any particular theory, these data suggest that CTHRC1 is able to form dimers and trimers. These data indicate that CTHRC1 is probably not glycosylated and it is therefore likely that the recombinant protein has similar properties as the native CTHRC1 which make it suitable for in vitro studies (described herein). The 28 kDa band in lane 1 probably represents reduced CTHRC1 (compare with FIG. 8, lanes 2 and 3).

Effects of CTHRC1 Overexpression in Xenopus laevis

The biological effects of CTHRC1 overexpression on Xenopus laevis development were studied using injection of CTHRC1 mRNA into frog embryos. For injection experiments, a dose of 5 ng of either the long form of CTHRC1 or the short form of CTHRC1 mRNA was injected into embryos at the 2 cell stage. Controls embryos were injected with an equal volume of empty vehicle or lacZ mRNA.

Injection of both CTHRC1_{L} and CTHRC1_{S} disturbed normal embryonic development. In general, the percentage of oocytes exhibiting disturbed development was significantly higher in the oocytes injected with the short form (close to 100%) while the long form of CTHRC1 showed fewer malformed embryos. Without wishing to be bound by any particular theory, these data may indicate that the short form of CTHRC1 is translated into protein in vivo.

In stage 17 embryos, there was a difference between lacZ-injected (FIG. 9A, left 2 embryos) and CTHRC1-injected embryos (FIG. 9A, right 2 embryos) indicating inhibition of blastopore closure.

At stage 34, control-injected embryos exhibited normal development (FIG. 9B), however, CTHRC1-injected embryos displayed a number of defects (FIG. 9C). The CTHRC1-injected embryos were smaller and were often distorted exhibiting abnormal development of the head. Due to failure of closure of the neural folds, fusion of the neurectoderm did not occur (FIG. 9D). Other malformations included development of a split tail (FIG. 9E). Several separate injection experiments were performed with similar results. This phenotype is remarkably similar to that of embryos injected with mRNA for dominant-negative FGF receptor constructs (Neilson and Friesel, 1996, J. Biol. Chem. 271:250497-25057).

The effect of CTHRC1 on mesoderm induction was further assessed in that CTHRC1 or control RNA was injected at the 2 cell stage and the embryos were allowed to develop to the blastula stage, at which time the animal pole ectoderm (animal caps) were dissected. Uninjected animal caps incubated in the presence of 200 ng/ml of FGF-1 elongated in a manner consistent with mesoderm induction while control-injected animal caps did not. Animal caps from embryos injected with CTHRC1 and incubated in the presence of FGF-1 resembled animal caps incubated without FGF-1. This indicates that CTHRC1 was able to block FGF-induced mesoderm formation.

Further, an important experiment assessed whether secretion of CTHRC1 is necessary for function as follows. Using PCR, a construct comprising a deletion of the first 32 amino acids that have the potential for being either a cleavable signal peptide or a transmembrane domain, was designed (SEQ ID NO:9, FIG. 10). The construct was cloned into the PCS2+ vector (American Type Culture Collection, Manassas, Va.) and RNA was injected into frog embryos at the 2 cell stage. The resulting phenotype was similar to the one seen in CTHRC1_{S}-injected embryos both in severity as well as frequency. Without wishing to be bound by any particular theory, these results suggest that it is likely that CTHRC1 is not a secreted protein and that the putative N-myristoylation sites can anchor the protein in the cell membrane if required for biological function(s).

Function of CTHRC1 in MC3T3 Cells

MC3T3 cells were transfected with control vector (pcDNA3.1 myc/his, Invitrogen, Carlsbad, Calif.) or with full length rat CTHRC1 cDNA in an antisense orientation. Stably transfected clonal cell lines were then obtained and used in cell proliferation assays.

MC3T3-E1 cells were transfected with an antisense CTHRC1 expression construct and stably transfected cell lines were established. Immunoblotting of clonal cell lines demonstrated that levels of CTHRC1 protein in antisense transfectants were undetectable, as compared with normal levels of CTHRC1 protein observed in vector transfectants (FIG. 11).

Morphology of the cells was determined using phase contrast microscopy for both vector transfected (FIGS. 12A through 12C) and antisense CTHRC1 transfected cells. As depicted in FIGS. 12D through 12I, antisense CTHRC1 transfected cells exhibited a distinctly altered phenotype demonstrating less adhesion to the substratum and reduced cell-cell contacts. More specifically, antisense transfected cells were elongated and fibroblastic in appearance (FIGS. 12D-I). This is in contrast to control vector transfected cells, which exhibited a cobblestone morphology (FIGS. 12D through 12I).

The data disclosed herein also demonstrate that there were increased numbers of dead cells and cell debris in the antisense transfected cells indicating, without wishing to be bound by any particular theory, increased cell turnover in CTHRC1 antisense transfected cells.

Increased cell turnover was determined by establishing growth curves of the clones (FIG. 13A) and measuring [³H]-thymidine incorporation in the clones (FIG. 13B) in parallel experiments. Even though cell counts were similar between control and antisense CTHRC1 transfected cells at all time points, [³H]-thymidine incorporation was significantly higher in the antisense CTHRC1 transfected cells at all time points examined (FIG. 13B). Increased cell turnover in the antisense transfected cells indicates that cell viability is reduced by shortening the cell life span. Together with the altered adhesion phenotype of the cells, the data suggests, without wishing to be bound by any particular theory, that CTHRC1 is involved in cell-matrix and cell-cell interaction(s).

Expression of CTHRC1 in Transgenic Mice

Transgenic mice were generated in which the coding region of CTHRC1 was under the control of the CMV promoter. Breeding of a CTHRC1 transgenic female with a CTHRC1 transgenic male gave rise to mouse pups that exhibited hemorrhaging in the hip and shoulder regions. The bleeding appeared to originate from the long bones (FIGS. 14A and 15A), as fractures of the humerus (FIG. 15B) and femur (FIG. 15C) were evident. In one instance, bleeding also occurred in smaller bones of the foot.

X-ray examination of the skeleton revealed that all transgenic mice were smaller than corresponding non-transgenic mice (FIG. 14B). This was particularly evident in the long bones.

Dissection of the dorsal skin revealed protrusion of the spinal cord similar to a phenotype seen in spina bifida disorders (FIG. 14C).

Skeletal preparations of the control and transgenic pups were prepared using standard methods in order to further investigate the skeletal abnormalities. The skeletal preparations revealed a striking decrease in Alcian blue staining affecting all sites of cartilage in transgenic mice. The spinal column (FIGS. 15D and 15E) revealed a virtual absence of Alcian blue staining. Alcian blue, which binds to proteoglycans, identifies the areas of cartilage formation. The lack of Alcian blue thus indicates a marked reduction in the proteoglycan content of the cartilage. Alcian blue staining was also reduced in the extremities, particularly the distal phalanges. The severity of the phenotype was variable, as in some cases, the offspring from a CTHRC1 transgenic/wildtype cross gave rise to severely affected mutants that died perinatally (FIGS. 15H through 15L). These mice were dwarfs with severe skeletal abnormalities affecting all bones (FIGS. 15H and 15I). In addition, separation of the skin occurred which is reminiscent of dystrophic epidermolysis bullosea (FIGS. 15H and 15L). X-ray examination of these transgenics showed extremely reduced bone density (lack of mineralization) and deformed bones (FIG. 15I). Masson's trichrome stained sections exhibited dramatically reduced collagen matrix (blue) in many organs including skull bone (FIG. 15J) and ribs (FIG. 15K). Separation of the epidermis from the dermis also appeared to result from a deficient collagen matrix (FIG. 15L). Preliminary analysis indicated that chondrocyte maturation appeared normal.

Mineralized bone of skeletal preparations was stained using Alizarin Red (pink color depicted as darker gray) and cartilage was stained using Alcian Blue (blue color depicted as lighter gray) (FIGS. 16A through 16J). The data disclosed herein demonstrate that there was a striking decrease in cartilage formation affecting all sites of cartilage generation including the extremities, particularly the distal phalanges (FIG. 16A compared with FIG. 16B). Cartilage was completely missing from the vertebra and intervertebral joints in the CTHRC1 transgenic mice (FIG. 16D) compared with normal pups (FIG. 16C), leaving the posterior parts of the vertebrae and the intervertebral joints without cartilage.

The absence of cartilage in the posterior parts of the vertebra surrounding the spinal cord is the most likely reason for the protrusion of the spinal cord leading the spina bifida phenotype. The anterior parts of the ribs exhibited strikingly reduced cartilage formation, which was most pronounced in the more caudal ribs (FIG. 16F compared with FIG. 16E).

Another finding was the marked decrease in the density of the mineralized bone which gave the flat bones of the skull a more transparent appearance (compare FIG. 16H with FIG. 16G). Without wishing to be bound by any particular theory, the decreased bone density is expected to result in weaker bones with increased tendency to fracture. Indeed, the hemorrhaging observed in the shoulder and hip regions was found to be the result of fractured long bones such as the humerus (FIGS. 161 and 16J) and femur.

For a better understanding of the phenotypes observed in CTHRC1 transgenic mice and to clarify whether the abnormalities were the result of ectopic expression or overexpression in cell types that have endogenous levels of CTHRC1, the sites of transgene expression were characterized and compared with the expression pattern of endogenous CTHRC1 protein. Polyclonal antibodies were generated in rabbits with the recombinant protein as antigen. One of the rabbits produced an antibody that recognized recombinant CTHRC1 and native CTHRC1 in lung tissue lysates on immunoblots with similar sensitivity and specificity as the peptide antibody described herein. In addition, the antibody was suitable for immunohistochemistry.

A protein A-purified IgG fraction was prepared from the antiserum and the corresponding preimmune serum was used to immunostain bone, skin and muscle sections from one day old normal and CTHRC1 transgenic pups. In parallel, immunostaining on sections with the transgene specific anti-myc antibody was performed (FIGS. 17 and 18). Chondrocytes of the humerus head express endogenous CTHRC1 mRNA (FIG. 17A) and endogenous CTHRC1 protein was expressed abundantly by chondrocytes (FIGS. 17B and 17C). However, CTHRC1 protein expression decreased sharply in the hypertrophic cartilage zone as it reaches the osteogenic front so that the bone matrix is essentially devoid of endogenous CTHRC1 protein (FIGS. 17B and 17C). Periosteal cells and osteoblasts expressed both CTHRC1 mRNA (FIG. 17D) and protein (FIG. 17F) although at a lower level than chondrocytes. Expression of endogenous CTHRC1 in osteocytes was not detectable (FIG. 17F). The CTHRC1 -myc transgene protein, however, was not expressed by chondrocytes (FIG. 17G, upper right corner) but high levels were synthesized by osteocytes in the bone matrix (FIG. 17H, arrowheads). Lower levels of CTHRC1 -myc protein were also found in osteoblasts. Controls for immunostaining were performed with preimmune IgG, and these revealed very little nonspecific background (FIG. 17I). Thus, in the transition from the cartilage matrix to the bone matrix, endogenous CTHRC1 and the CTHRC1 -myc transgene had inverse expression patterns (compare FIGS. 177B and 17G). Without wishing to be bound to any particular theory, the endogenous CTHRC1 expression pattern supports the idea that CTHRC1 functions as an inhibitor of mineralization, and the expression pattern of the transgene provides an explanation for the normal development of the cartilage in the transgenic mice. With regard to osteoblasts, the transgenic mice provide an in vivo overexpression model.

Immunohistochemistry performed on skin sections from normal one day old mouse pups showed no CTHRC1 protein detectable by the anti-CTHRC1 IgG (FIG. 18A). It should be emphasized, however, that skin wound repair is associated with high levels of CTHRC1 mRNA expression by dermal fibroblasts. High levels of the CTHRC1 -myc transgene were expressed by keratinocytes in the epidermis as determined by immunohistochemistry with the anti-myc antibody (FIG. 18B). The dermis of these transgenic mice was characterized by an unusually loose connective tissue (FIG. 18C) and hair follicles were often absent or poorly developed. The abnormalities in the skin could explain the DEB phenotype. Skeletal muscle in normal mice revealed CTHRC1 immunoreactivity in the muscle fibers (FIG. 18D) and high levels of the transgene were also detected in skeletal muscle with the anti-myc antibody (FIG. 18G and FIG. 17G). Interestingly, the muscle fiber bundles in the transgenics were often shorter and less densely packed (FIGS. 18H and 18I). In addition, the fiber bundles often formed circular structures that appeared hollow in the center (FIGS. 18G, 18H, and 18I). These findings indicate the presence of a severe myopathy in CTHRC1 transgenic mice. With regard to skeletal muscle, the transgenic mice provide an in vivo overexpression model. Expression of the transgene in skeletal muscle is consistent with data presented herein that demonstrates activity of the CMV promoter in this tissue.

The data disclosed herein using transgenic mice indicate that CTHRC1 plays an important role in bone growth. Similar to the frog embryo injection experiments wherein CTHRC1 mRNA mediated a failure of dorsal closure, the mouse transgenics also exhibit spina bifida-like defects of the spinal column.

These findings demonstrate that altered expression of CTHRC1 is affecting vital mechanisms of bone formation. In particular, increased CTHRC1 expression inhibits cartilage and bone formation resulting in reduced bone growth and bone mineralization which gives rise to more fragile bones. Furthermore, without wishing to be bound by any particular theory, the data disclosed herein suggest that inhibition of CTHRC1 expression can lead to the opposite phenotype with increased cartilage formation and increased bone density and strength. The level of CTHRC1 expression could thus be a predictor of bone formation, bone density and bone strength. Further, inhibiting CTHRC1 expression may be useful for diseases, disorders, or conditions associated with decreased bone density, bone formation and bone strength such as, but not limited, osteoporosis, and the like.

The data disclosed herein demonstrate that CTHRC1 is expressed selectively in settings where remodeling occurs, i.e., skin incisional wounds, bone, and the like. Without wishing to be bound by any particular theory, these data suggest that the role of CTHRC1 is not restricted to the vasculature but instead CTHRC1 expression is be relevant to events in wound healing in general, including bone formation, bone density and bone strength.

Wound healing is characterized by the formation of granulation tissue from connective tissue surrounding the damaged area and its components are inflammatory cells, fibroblasts and myofibroblasts (smooth muscle α-actin positive). As the wound closes and evolves into a scar, there is an important decrease in cellularity and a specific disappearance of myofibroblasts (Ducy et al., 2000, Science 289:1501-1504; Giachelli et al., 1993, J. Clin. Invest. 92:1686-1696). This cell loss has been shown to occur by apoptosis (Giachelli et al., 1993, J. Clin. Invest. 92:1686-1696). Failure to decrease this cellularity may contribute to hypertrophic scarring and keloid formation (Balica et al., 1997, Circulation 95:1954-1960; Bostrom et al., 1993, J. Clin. Invest. 91:1800-1809). The response of the adventitia to balloon injury (Bostrom et al., 1995, Amer. J. Cardiol. 75:88B-91B), and the response of the myocardium to infarction are very similar (Luo et al., 1997, Nature 386:78-81) with early accumulation of myofibroblasts and subsequent loss of cells by apoptosis as disclosed elsewhere herein resulting in an acellular matrix-rich structure. It should be emphasized that, as demonstrated by data disclosed elsewhere herein, CTHRC1 is induced in these myofibroblasts while it is not expressed in the dedifferentiated smooth muscle cells (SMC) of the neointima. While smooth muscle α-actin is down-regulated in the dedifferentiated, proliferating SMC of the neointima, it is induced in the myofibroblasts of the adventitia (Bostrom et al., 1995, Amer. J. Cardiol. 75:88B-91 B), but is lost from the adventitial cells within 2 weeks after injury.

CTHRC1 expression may play a role in other clinically relevant situations of fibrosis, including liver fibrosis and pulmonary fibrosis. In liver fibrosis, apoptosis of hepatic stellate cells has been implicated in the fibrotic process and targeting apoptosis may be a promising strategy for antifibrotic therapies (Cales et al., 1998, Biomed. Pharmacother. 52:259-263). TGF-β has been identified as the major factor responsible for fibrosis in the bleomycin-induced lung fibrosis model Wang et al., 1999, Thorax 54:805-812). Finally, since CTHRC1 is expressed in developing bone it should be mentioned that apoptosis is an integral part of endochondral ossification and bone fracture healing with chondrocytes and osteoblasts undergoing apoptosis (Einhorn et al., 1998, Clin. Orthop. S7-21; Olmedo et al., 1999, J. Orthop. Trauma 13:356-362). The prominent expression of CTHRC1 in developing bone further suggests, without wishing to be bound by any particular theory, that CTHRC1 is involved in regulating calcification since inhibition of calcification is an important event in cell death (Kim, 1995, Scanning Microsc. 9:1137-1178; Kockx et al, 1998, Arterioscler. Thromb. Vasc. Biol. 18:1519-1522).

As discussed herein, mutations in collagens are responsible for OI, Bethlem myopathy, and DEB (reviewed in Spranger et al., 1994, Eur. J. Pediatr., 153:56-65). Therefore, a reasonable hypothesis is that CTHRC1 functions as a modulator of collagen matrices. Data presented herein make it clear that it is absolutely essential that the CTHRC1 expression levels be tightly regulated for normal development. The importance of this molecule is further demonstrated in that it is the first non-collagen gene to produce an OI phenotype. Based on data presented herein, it is likely that CTHRC1 is an endogenous modulator of collagen matrices. In that capacity, the molecule could function as an endogenous regulator of (anti)fibrotic responses. The inhibition of bone mineralization could be a consequence of a deficient collagen bone matrix that does not support mineralization. In addition, the potential role of CTHRC1 as a signaling molecule needs to be addressed as it was able to inhibit osteopontin promoter activity and Cbfal-dependent activity of the osteocalcin promoter. Furthermore, reduced expression of collagen was seen in cells overexpressing CTHRC1 (FIG. 6). If CTHRC1 would function solely by incorporation as a component of a collagenous matrix, it should be present in matrices of healed wounds. A completed repair process in response to angioplasty injury of an artery, however, is associated with low CTHRC1 levels (FIG. 5C, 28 day time point) similar to normal vessels.

In summary, the hypothesis of CTHRC1 as a modulator of collagen matrices is based on the following findings: 1) increased CTHRC1 expression in vitro is associated with decreased TGF-β expression as well as reduced TGF-β dependent gene expression (collagens I and III) in vivo and in vitro, 2) increased CTHRC1 expression in vivo results in phenotypes reminiscent of collagen mutations found in OI, DEB, and myopathies, 3) in the absence of CTHRC1, expression of bone differentiation markers such as osteopontin and ALP are dramatically increased, and 4) the presence of a 36 aa domain with homology to the triple helical repeat region of collagens. The proposed hypothesis for the function of CTHRC1 is based on insight derived mainly from in vivo overexpression as well as ectopic expression.

### Example 2

### Antifibrotic Properties of CTHRC1 Relating to Fibrosis and Restenosis

The data disclosed previously elsewhere herein demonstrate, inter alia, that CTHRC1 is a novel nucleic acid sequence encoding a protein expressed in arteries following balloon catheter injury. No expression of CTHRC1 was detected in normal arteries. However, in injured arteries, CTHRC1 was expressed by smooth muscle cells proliferating in the neointima, as well as by fibroblasts in the adventitia undergoing fibrosis. The data further demonstrate that transgenic mice overexpressing CTHRC1 under CMV promoter control exhibited bleeding (FIGS. 15A and 20A), which bleeding originated from fractured bones (FIGS. 15C and 20B). The brittleness of the bones was due to a reduction in bone matrix in the transgenic mice (FIGS. 20C and 20D), and the majority of the bone matrix consists of collagen type I.

The association of increased CTHRC1 expression levels with decreased collagen matrix deposition (bone matrix) prompted investigation of the role of CTHRC1 in disease conditions accompanied by fibrosis caused by excessive collagen matrix deposition. In particular, collagen matrix production in mouse carotid arteries in an art-recognized model of intimal hyperplasia and constrictive remodeling (Kumar and Lindner, 1997, Arterioscler. Thromb. Vasc. Biol. 17:2238-2244) was examined. This was done in wildtype/control mice as well as in transgenic mice overexpressing CTHRC1. Also, the amount of collagen production in a smooth muscle cell line (PAC1) overexpressing CTHRC1 protein was assessed and compared to the same cell line in which endogenous CTHRC1 expression was inhibited by stably expressing the CTHRC1 sequence in reverse orientation (antisense transfectants). In addition, the role of CTHRC1 in an art-recognized renal fibrosis model was examined by comparing the degree of fibrosis in wildtype mice compared with transgenic mice overexpressing CTHRC1. The data disclosed herein demonstrate that expression of CTHRC1 inhibits formation of collagen matrix and, therefore, provides a method for inhibiting fibrosis, restenosis, or both, associated with, or mediated by, collagen matrix formation.

Overexpression of CTHRC1 in transgenic Mice is Associated with Decreased Collagen Production in Bone

The apparent decrease in bone matrix observed in CTHRC1 transgenic mice was examined by Northern blot and immunoblot analysis performed on skull bones obtained from wildtype and CTHRC1 transgenic mouse pups. Collagen I mRNA levels in transgenic skull bones were increased while collagen I protein levels in the same samples were decreased (FIG. 21) compared to controls. Increased collagen I mRNA levels were also observed in C3H10T/½ cells overexpressing CTHRC1 (FIG. 21, right side of panel). Thus, collagen I protein levels were reduced despite increased mRNA levels.

Overexpression of CTHRC1 in PAC 1 Smooth Muscle Cells Leads to Reduced Collagen Production and Cell Migration

PAC1 smooth muscle cells express basal levels of endogenous CTHRC1. Stable PAC1 CTHRC1 antisense transfectants were established and this led to a near complete inhibition of detectable CTHRC1 protein expression (FIG. 22). Quantification of procollagen protein in CTHCR1 overexpressing PAC1 cells was compared to untransfected control PAC1 cells and CTHRC1-antisense transfected PAC1 cells (PAC 1-AS) (FIG. 23). This was done by immunoblot analysis using a procollagen antibody (clone SP1.D8, Developmental Studies Hybridoma Bank, University of Iowa, Iowa City, Iowa). For evaluation of protein loading the same samples were also immunoblotted with an anti-actin antibody (Sigma) (FIG. 23). Procollagen levels were markedly reduced in the cell lysate as well as in the conditioned medium (“CM”) of CTHRC1 overexpressing cells.

Overexpression of Wildtype CTHRC1 is Associated with Reduced Collagen type I mRNA Expression

Stably transfected PAC1 smooth muscle cell lines were produced that over-expressed one of the following: wildtype CTHRC1 (PAC1-Cthrc1); a mutant Cthrc1 (PAC1-mCol) wherein the collagen domain of human CTHRC1 (comprising the amino acid sequence GPAGVPGRDGSPGANGIPGTPGIPGRDGFKGEKG [SEQ ID NO: 14] encoded by the nucleic acid sequence ggaccagcaggagttcctggtcgcgatgggagccctggggccaatggcattcctggcacaccgggaatcccaggtcg ggatggattcaaaggagagaaaggg [SEQ ID NO:15]) was replaced with a collagen domain of equal length obtained from mouse collagen 1 alpha1 (comprising amino acid sequence GSPGTAGARGNDGAVGAAGPPGPTGPTGPPGFGF [SEQ ID NO:16], encoded by nucleic acid sequence ggatcccctggcactgctggtgctc gcggtaacga tggtgctgtt ggtgctgctg gaccccctgg tcccaccggccccactggcc ctcctggcttcggattc [SEQ ID NO:17]); and antisense Cthrc1 (PAC1 -Cthrc1AS) described elsewhere herein. Non-transfected PAC1 cells (PAC1-Control) were also used as controls. Northern blots of mRNA derived from each cell line were probed with radiolabeled mouse collagen 1 alpha1 (colla1), rat collagen 1 alpha2 (colla2), as well as with GAPDH as an internal loading control (FIG. 24). Note that two transcripts were detectable for both collagen 1 alpha1 and collagen 1 alpha2 in all cells except those transfected with PAC1 -Cthrc1. The data disclosed herein demonstrate that overexpression of wildtype Cthrc1, but not expression of mutant CHTRC1 (mCol) or antisense CTHRC1 (AS), was associated with profound down-regulation of all collagen 1 mRNA transcripts. That is, relative collagen mRNA levels in the other transfected cell lines were similar to those observed in control non-transfected PAC1 cells. These data also indicate that the ability of CTHRC1 to down-regulate collagen mRNA expression is dependent upon its native “GXY” repeat.

Overexpression of CTHRC1 in PAC1 Cells is Associated with Reduced Cell Migration

A scratch wound assay was performed on confluent CTHRC1 and CTHRC1 -antisense transfected PAC I cells to determine the effects of CTHRC1 on cell migration (FIG. 25). Coverage of the scraped area with migrating cells was quantified by planimetry of digital images at the indicated times after scrape wounding and the results were graphically depicted in FIG. 26. PAC1 cells overexpressing CTHRC1 demonstrated significantly delayed closure of the scraped area compared with PAC1 cells transfected with CTHRC1 antisense (FIGS. 25 and 26).

Collagen Deposition and Adventitial Fibrosis are Reduced in CTHRC1 Transgenic Mice

Ligation of the left common carotid artery was performed as previously described by Kumar and Lindner (1997, Arterioscler. Thromb. Vasc. Biol. 17:2238-2244). This procedure leads to formation of an intimal lesion and also to adventitial fibrosis. Histological analysis was performed on cross-sections of carotid arteries from wildtype mice and on carotid arteries obtained from CTHRC1 transgenic mice 2 weeks after carotid artery ligation (FIGS. 27A-D). For visualization of collagen, sections were stained with Masson's Trichrome which stains collagen blue (which is depicted as darker gray herein). In vessels from wildtype mice, abundant deposition of collagenous matrix was evident in the intima and media (FIGS. 27A and 27B), while very little collagen matrix was detectable in the intima and media of CTHRC1 transgenic mice (FIGS. 27C and 27D). The transgenic vessel shown in FIG. 27D revealed marked medial hyperplasia instead of the typical intimal hyperplasia detected in wildtype mice carotids. The data disclosed demonstrate that CTHRC1 can reduce restenosis and constrictive vessel remodeling thereby providing important novel therapeutics relating to such conditions.

Collagen Deposition is Reduced in CTHRC1 Transgenic Mice in a Renal Fibrosis Model

The renal fibrosis model of unilateral ligation and dissection of the ureter described in Diamond (1995, Am. J. Kidney Dis. 26:133-140) was applied to wildtype and CTHRC1 transgenic mice to evaluate the effects of elevated CTHRC1 levels on the development of fibrosis. Collagen deposition was evaluated on Masson's Trichrome stained sections obtained 2 weeks after ligating and dissecting the left ureter. Briefly, groups of wildtype and CTHRC1 transgenic mice (n=5 animals per group) had their left ureter ligated and dissected. Groups of animals were sacrificed at 4, 7, and 14 days after ureter ligation. CTHRC1 transgenic mice showed much reduced fibrosis evident by the reduced amount of collagenous matrix (blue staining depicted herein as dark gray) (FIG. 27E, F).

The left kidney was removed and either processed for histology as described above or flash frozen for immunoblotting. For immunoblotting, lysates were prepared from the kidneys and 100 microgram protein per sample was subjected to SDS-PAGE followed by immunoblotting with anti-collagen type I (Calbiochem) or anti-myc antibody (Zymed).

The amount of collagen type I production in the kidneys 4 days following ureter obstruction was quantified by immunoblot analysis of kidney tissue lysates (FIG. 28). The data disclosed herein demonstrate that collagen type I production was substantially reduced in CTHRC1 transgenic mice compared to wildtype control mice.

CTHRC1 is Cleaved by Matrix Metalloprotease-1 (MMP-1) and Collagenase

The schematic representation of the CTHRC1 protein demonstrates a short collagen domain with a glycine (G) residue in every third position near the N terminus (at about amino acids 60-95) (FIG. 29). In collagen molecules, this domain is responsible for triple helix formation. Cross-linking of CTHRC1 with disuccinimidyl octanedioate (DSS) prior to immunoblotting with anti-CTHRC1 antibody demonstrates CTHRC1 running at a molecular weight consistent with a trimer (FIG. 29, bottom of figure, immunoblot lane 3).

The CTHRC1 collagen domain is susceptible to cleavage by bacterial collagenase (FIG. 30). In addition, MMP-1 cleaves native collagen type I at a single site within the collagen domain where a glycine residue is followed by an isoleucine; therefore, cleavage of CTHRC1 by collagenase was examined. The data disclosed herein demonstrate that highly purified bacterial collagenase (Sigma) (FIG. 30), as well as MMP-1 (FIG. 31) cleave CTHRC1 near the N terminus. MMP-7 and thrombin do not cleave CTHRC1 (FIG. 31), demonstrating that cleavage of CTHRC1 by MMP-1 is specific. The amino acid sequence of CTHRC1 cleaved by MMP-1 to produce a longer cleavage fragment (cCTHRC1_{L}) is set forth in FIG. 32B (SEQ ID NO:11) and the nucleic acid sequence corresponding to this longer cleavage fragment is set forth in FIG. 32A (SEQ ID NO:10). Similarly, cleavage at the second MMP-1 cleavage site results in a cleaved CTHRC1 shorter fragment polypeptide (cCTHRC1_{S}) comprising the amino acid sequence set forth in FIG. 33B (SEQ ID NO:13), and the nucleic acid sequence corresponding to the amino acid sequence is set forth at FIG. 33A (SEQ ID NO:12).

These data support that the collagen domain within the CTHRC1 protein is responsible for trimer formation and that CTHRC1 is susceptible to cleavage by MMP-1. The latter appears to be of biological significance since the vast majority of endogenous CTHRC1 in PAC1 cells migrates as fragments with reduced molecular weight compared to full-length CTHRC1 purified from CHO cells (FIG. 22, especially as indicated by the arrows).

As demonstrated previously elsewhere herein, PAC1 cells stably transfected with an expression construct of wildtype CTHRC1 can be maintained in culture. These transfectants display diminished cell migration. However, transfection of PAC1 cells with a mutant CTHRC1 in which the entire collagen domain was deleted compromised the viability of the cells and stable transfectants comprising this mutant CTHRC1 could not be established. All cells died under selection with G418 and, without wishing to be bound by any particular theory, this mutant CTHRC1 likely does not form a trimer. Without wishing to be bound by any particular theory, the presence of endogenous CTHRC1 in a cleaved form in cells, the ability of MMP-1 to cleave CTHRC1, and the profound effects of a mutant CTHRC1 lacking the collagen domain, support that CTHRC1 may require cleavage for its activity. Thus, the activity of CTHRC1 may depend on the presence of MMP-1 or another protease. Further, the data suggest that inhibition of cleavage by MMP-1, such as, but not limited to, by matrix metalloprotease inhibitors (MMP1) that inhibit MMP-1, can inhibit CTHRC1 function by inhibiting cleavage necessary for CTHRC1 biological function.

The data disclosed herein demonstrate that the ability to inhibit collagen matrix production with CTHRC1 can be beneficial in a variety of clinical conditions characterized by excessive collagen production. These conditions include, but are not limited to, scarring and fibrotic processes as in scarring of wounds, skin wounds, keloid formation, scarring associated with chronic inflammatory processes such as in renal fibrosis, pulmonary fibrosis, and vascular fibrosis associated with angioplasty procedures leading to restenosis.

Alternatively, the data disclosed herein demonstrate that decreasing CTHRC1 expression can result in enhanced collagen matrix production, which may be desirable in certain situations where the strength of the connective tissue should be increased. Examples of beneficial uses relating to increased CTHRC1 in a cell or tissue include, but are not limited to, any form of wound healing where increase tensile strength is desired in a tissue or organ (e.g., bone, skin, tendons and ligaments, etc.). Additionally, since plaque rupture is the major cause of heart attacks, a plaque with a stable fibrous cap is considered beneficial. Thus, a fibrous cap with increased strength can prevent plaque rupture and decreasing levels of CTHRC1 in this disease condition may be beneficial since increased CTHRC1 can prevent such rupture.

### Example 3

### CTHRC1 Regulation of BMP1

Bone morphogenetic protein 1 (BMP1) is involved in the processing of fibrillar collagens type I, II, and III by cleaving the C terminal propeptide. It has also been found to cleave the propeptide of lysyl-oxidase, an enzyme involved in cross-linking of collagen fibrils. Cleavage of these propeptides is important for proper collagen formation. BMP1 also degrades chordin, which functions as an antagonist of bone morphogenetic proteins.

Transgenic mice overexpressing CTHRC1 under CMV promoter control exhibited bleeding (FIG. 20A) which originated from fractured bones (FIG. 20B). The brittleness of the bones was due to a reduction in bone mass in the transgenic mice (FIG. 20C and 20D).

Overexpression of CTHRC1 in C3H10T½ cells as shown by immunoblot analysis with anti-CTHRC1 antiserum (FIG. 34C) is associated with a decrease in BMP1 mRNA levels (FIG. 34A). In the skin of CTHRC1 transgenic mice, BMP1 protein levels are decreased (FIG. 35). This decrease in BMP1 is associated with an increase chordin protein levels in the skin (FIG. 35). Therefore, without wishing to be bound by any particular theory, the brittle bones with reduced bone matrix formation observed in CTHRC1 transgenic mice can be caused by inhibition of collagen deposition due to reduced processing of collagen proforms caused by the lack of BMP1.

The apparent decrease in bone matrix observed in CTHRC1 transgenic mice was examined by Northern blot and immunoblot analysis performed on skull bones obtained from wildtype and CTHRC1 transgenic mouse pups. Collagen I mRNA levels in transgenic skull bones were increased (FIG. 21A) while collagen I protein levels in the same samples were decreased (FIG. 21C) compared to controls. Increased collagen I mRNA levels were also observed in C3H10T½ cells (these are same cells as shown in bottom panel of FIG. 34) overexpressing CTHRC1 (FIG. 21B). Thus, collagen I protein levels were reduced despite increased mRNA levels. In the presence of decreased BMP1 expression, this is likely to be caused by reduced processing of procollagen by BMP1. This would also explain the reduced amount of bone matrix found in CTHRC1 transgenic mice.

The ability to inhibit collagen matrix production with CTHRC1 can be beneficial in a variety of clinical conditions characterized by excessive collagen production. Such conditions include, but are not limited to, scarring and fibrotic processes as in scarring of wounds, skin wounds, keloid formation, scarring associated with inflammatory processes, pulmonary fibrosis, and vascular fibrosis associated with angioplasty procedures leading to restenosis, negative remodeling, and the like. Thus, administration of CHTRC 1 to inhibit BMP1 -mediated processes (e.g., processing of various propeptides, chordin expression, and the like) can provide a therapeutic benefit where reduced collagen deposition, bone matrix formation, and the like, are desired, such as, but not limited to, treatment of the afore-mentioned conditions.

One skilled in the art, based upon the disclosure provided herein, would understand that methods for administering CTHRC1 encompass not only administering the protein, but also administering a nucleic acid encoding the protein, and such methods are more fully discussed elsewhere herein. Thus, once armed with the teachings provided herein, the skilled artisan would understand the increasing CTHRC1, by administering the protein and/or a nucleic acid encoding the protein, can provide a benefit and can be performed according to the novel methods provided herein.

Increasing CTHRC1 expression can be beneficial for mediating decreased collagen matrix production. Based upon the disclosure provided herein, the skilled artisan would appreciate that increasing collagen matrix production is desirable in certain situations. Alternatively, increased collagen matrix formation is desired, for instance, where the strength of the connective tissue should be increased. Examples include, but are not limited to, any form of wound healing to increase tensile strength (bone, skin, tendons and ligaments, etc).

Further, since plaque rupture in a blood vessel is the major cause of heart attacks, a plaque with a stable fibrous cap is potentially beneficial. Thus, a fibrous cap with increased strength can prevent plaque rupture such that decreasing levels of CTHRC1 in this disease condition can be beneficial. This is because, as disclosed for the first time herein, decreasing CTHRC1 can increase BMP1 expression and the level of BMP1 protein in cell, thereby increasing propeptide processing to produce collagen and lysyl-oxidase, and increasing collagen deposition, thereby stabilizing the plaque such that it does not rupture and cause subsequent damage (e.g., myocardial infarction, and the like).

Inhibition of CTHRC1 can be accomplished as more fully set forth previously elsewhere herein. That is, methods for inhibiting CTHRC1 expression or function include use of antisense nucleic acids, intrabodies, and the like, as would be well understood by one skilled in the art. Therefore, where inhibition of CTHRC1 is desired, the skilled artisan, armed with the teachings provided herein, would be able to inhibit CTHRC1 thereby mediating the effects disclosed herein, e.g., increasing the level of BMP1 mRNA, and increasing the level of BMP1 in a cell, decreasing the level of chordin in a cell, among others. The skilled artisan would understand, based upon the disclosure provided herein, a condition, disease or disorder where providing these benefits using the novel methods disclosed herein would treat or ameliorate the disease, disorder or condition.

### Example 4

### CTHRC1 in Regulation of BMP4

Stably transfected MC3T3-E1 cell lines were established expressing full length complementary rat CTHRC1 mRNA (cRNA), hereinafter referred to as “MC3T3-E1 Cthrc1 antisense”. As controls, MC3T3-E1 cell lines were established with an empty vector (“MC3T3-E1 vector”). In both cases, the mammalian expression vector pcDNA3.1 (Invitrogen) was used.

C3H10T/½ cells were transduced using a retroviral expression construct expressing the full length coding sequence of rat CTHRC1 (“C3H10T½ Cthrc1”. The control C3H10T/½ cells were transduced with a retroviral vector containing an empty vector (“C3H10T½ vector”).

Immunoblot analysis using anti-CTHRC1 antibody of cell lysates from the MC3T3-E1 cell lines revealed that the antisense transfected cell line had markedly reduced levels of CTHRC1 compared with control cells transfected with an empty vector (FIG. 36, left middle panel). The reduction in CTHRC1 expression was paralleled by an equally dramatic reduction of BMP4 mRNA as determined by Northern blot analysis.

C3H10T½ vector control cells, on the other hand, did not express CTHRC1 mRNA, nor did they express BMP4 mRNA as determined by Northern blotting (FIG. 36). Overexpression of CTHRC1 mRNA in C3H10T½ Cthrc1 cells, however, coincided with an induction of BMP4 mRNA in those cells. These data demonstrate that the expression levels of BMP4 closely follow those of CTHRC1 and furthermore, these data demonstrate that CTHRC1 can regulate the expression level of BMP4. These data provide a surprising, novel mechanism for regulation of BMP4 in a cell.

Analysis of skull bones obtained from wildtype mice and from CTHRC1 transgenic (“Tg”) mice overexpressing CTHRC1 mRNA (i.e., Tg15 and Tg32) demonstrated that levels of BMP4 were also increased in vivo due to overexpression of CTHRC1 (FIG. 37). This increase in BMP4 coincided with an increase in transcription of Msx1 mRNA (FIG. 37). Msx1 is the transcription factor muscle segment homeobox gene 1, which is known to be regulated by BMP4.

The data disclosed herein also demonstrate that osteopontin mRNA levels in CTHRC1 transgenic mouse bones where CTHRC1 was overexpressed were reduced compared to wildtype mice expressing normal amount of CTHRC1 (FIG. 37).

Similarly to the results obtained in vivo, the data disclosed herein demonstrate an inverse relationship between CTHRC1 expression and osteopontin expression was also observed in antisense CTHRC1 transfected MC3T3-E1 cells compared to controls (FIG. 37, left panel). That is, in MC3T3-E1 Cthrc1 antisense cells, where CTHRC1 expression is inhibited by an antisense molecule, OPN expression was increased.

BMP4 plays critical roles in many organ systems and in cell differentiation processes. For instance, the development of the central nervous system and skeletal systems depends upon BMP4, as does the differentiation of stem cells. BMPs are also used to promote bone and cartilage formation in orthopedic settings of fracture healing and bone/cartilage repair. While not wishing to be bound by any particular theory, the use of CTHRC1 to increase BMP4 expression can provide an important therapeutic benefit for promoting bone and cartilage growth in any orthopedic application where skeletal repair/healing is sought and in promoting the differentiation of stem cells into different cell lineages.

One skilled in the art would, based upon the disclosure provided herein, understand that reducing the level of CTHRC1 expression can be important in the downregulation of BMP4, which provides a significant advantage in a situation where it is desired to maintain stem cells in an undifferentiated state. That is, the skilled artisan would appreciate, once armed with the teachings provided herein, that the present invention provides a novel method of regulating BMP4 expression and/or function by regulating the level of CTHRC1 both in vitro and in vivo. Thus, where expression and/or function of BMP4 is desired, CTHRC1 expression can be increased which then induces and/or increases the level of BMP4. Alternatively, where it is desirable to decrease the level of BMP4, the level of CTHRC1 can be decreased, mediating the desired effect.

Likewise, the level of Msx1 can also be regulated by increasing or decreasing the level of CTHRC1 in a cell. That is, as demonstrated by the data disclosed herein, increasing the level of CTHRC1 mediates an increase in the level of Msx1 and decreasing the level of CTHRC1 mediates a decrease in the level of Msx1 in a cell. Thus, by regulating or affecting the level of CTHRC1 in a cell, the level of Msx1 can also be regulated and/or affected. The skilled artisan would readily appreciate, based upon the disclosure provided herein, a condition, disease or disorder where regulation of Msx1, either to decrease or increase the level of Msx1 in a cell or tissue, would be desirable.

Further, the data disclosed herein demonstrate that expression of CTHRC1 mediates a decrease in the level of osteopontin. Thus, where it is desirable to decrease the level of OPN, the level of CTHRC1 can be increased. Alternatively, where it is desirable to increase the level of OPN, the level of CTHRC1 can be decreased. The skilled artisan, based upon the disclosure provided herein, would appreciate situations where an increased or decreased level of osteopontin would be useful.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated nucleic acid encoding a human cleaved CTHRC1.

2. The isolated nucleic acid of claim 1, wherein said nucleic acid shares at least about 33% sequence identity with a nucleic acid encoding at least one of cleaved CTHRC1 longer fragment (SEQ ID NO:10), and a human cleaved CTHRC1 shorter fragment (SEQ ID NO:12).

3. An isolated nucleic acid encoding a human cleaved CTHRC1, wherein the amino acid sequence of said human cleaved CTHRC1 shares at least about 33% sequence identity with an amino acid sequence of at least one of SEQ ID NO:11, and SEQ ID NO:13.

4. An isolated polypeptide comprising a human cleaved CTHRC1.

5. The isolated polypeptide of claim 4, wherein said human cleaved CTHRC1 shares at least about 6% sequence identity with an amino acid sequence of at least one of SEQ ID NO:11, and SEQ ID NO:13.

6. The nucleic acid of claim 1, said nucleic acid further comprising a nucleic acid encoding a tag polypeptide covalently linked thereto.

7. The nucleic acid of claim 6, wherein said tag polypeptide is selected from the group consisting of a green fluorescent protein tag polypeptide, an influenza virus hemagglutinin tag polypeptide, a myc tag polypeptide, a glutathione-S-transferase tag polypeptide, a myc-pyruvate kinase tag polypeptide, a His6 tag polypeptide, a FLAG tag polypeptide, and a maltose binding protein tag polypeptide.

8. The nucleic acid of claim 1, said nucleic acid further comprising a nucleic acid specifying a promoter/regulatory sequence operably linked thereto.

9. A vector comprising the nucleic acid of claim 1.

10. The vector of claim 9, said vector further comprising a nucleic acid specifying a promoter/regulatory sequence operably linked thereto.

11. A recombinant cell comprising the isolated nucleic acid of claim 1.

12. A recombinant cell comprising the vector of claim 9.

13. An isolated nucleic acid complementary to the nucleic acid of claim 1, said complementary nucleic acid being in an antisense orientation.

14. The isolated nucleic acid of claim 13, wherein said nucleic acid shares at least about 33% identity with a nucleic acid complementary with a nucleic acid having the sequence of at least one of a human cleaved CTHRC1 longer fragment (SEQ ID NO:10), and a human cleaved CTHRC1 shorter fragment (SEQ ID NO:12).

15. A recombinant cell comprising the isolated nucleic acid of claim 13.

16. An antibody that specifically binds with a human cleaved CTHRC1 of claim 5.

17. The antibody of claim 16, wherein said antibody is selected from the group consisting of a polyclonal antibody, a monoclonal antibody, a humanized antibody, a chimeric antibody, and a synthetic antibody.

18. A composition comprising the isolated nucleic acid of claim 13 and a pharmaceutically-acceptable carrier.

19. A composition comprising the isolated nucleic acid of claim 1 and a pharmaceutically-acceptable carrier.

20. A composition comprising the isolated polypeptide of claim 4 and a pharmaceutically-acceptable carrier.

21. A transgenic non-human mammal comprising the isolated nucleic acid of claim 1.

22. A method of treating a disease mediated by collagen matrix production in a human in need thereof, said method comprising administering to a human afflicted with said disease an effective amount of CTHRC1, thereby treating said disease mediated by collagen matrix production in said human.

23. The method of claim 22, wherein said CTHRC1 is administered as a molecule selected from the group consisting of a CHTRC I polypeptide and a nucleic acid encoding CTHRC1.

24. The method of claim 23, wherein said disease is selected from the group consisting of fibrosis, constrictive remodeling, and restenosis.

25. The method of claim 24, wherein said fibrosis is fibrosis of an organ.

26. The method of claim 25, wherein said organ is at least one organ selected from the group consisting of kidney, lung, liver and skin.

27. A method of treating constrictive remodeling in a human in need therefor, said method comprising administering to a human an effective amount of CTHRC1, thereby treating said constrictive remodeling in said human.

28. A method of preventing constrictive remodeling in a human in need therefor, said method comprising administering to a human an effective amount of CTHRC1, thereby preventing said constrictive remodeling in said human.

29. A method of treating restenosis in a human in need therefor, said method comprising administering to a human an effective amount of CTHRC1, thereby treating restenosis in said human.

30. A method of preventing restenosis in a human in need therefor, said method comprising administering to a human an effective amount of CTHRC1, thereby preventing restenosis in said human.

31. A method of treating fibrosis in a human in need therefor, said method comprising administering to a human an effective amount of CTHRC1, thereby treating fibrosis in said human.

32. A method of preventing fibrosis in a human in need therefor, said method comprising administering to a human an effective amount of CTHRC1, thereby preventing fibrosis in said human.

33. A kit for treating a disease mediated by collagen matrix formation in a human in need therefor, said kit comprising an effective amount of CTHRC1, said kit further comprising an applicator, and an instructional material for the use thereof.

34. The kit of claim 33, wherein said disease is selected from the group consisting of constrictive remodeling, arterial restenosis, vessel injury, and fibrosis.

35. A kit for preventing a disease mediated by collagen matrix formation in a human in need therefor, said kit comprising an effective amount of CTHRC1, said kit further comprising an applicator, and an instructional material for the use thereof.

36. An isolated nucleic acid encoding a mutant CTHRC1, wherein said nucleic acid comprises a nucleotide sequence encoding a human CTHRC1 collagen domain is replaced by a nucleotide sequence of mouse collagen 1 alpha 1 encoding a mouse collagen 1 alpha 1 collagen domain.

37. The isolated nucleic acid of claim 36, wherein said nucleotide sequence encoding said human CTHRC1 collagen domain is SEQ ID NO:15 and further wherein said nucleotide sequence encoding said mouse collagen 1 alpha 1 collagen domain is SEQ ID NO:17.

38. An isolated mutant CTHRC1 polypeptide, wherein said polypeptide comprises substitution of a human CTHRC1 collagen domain with a mouse collagen 1 alpha 1 collagen domain.

39. The isolated polypeptide of claim 38, wherein the amino acid sequence of said human CTHRC1 collagen domain is SEQ ID NO:14 and further wherein the amino acid sequence of said mouse collagen 1 alpha 1 collagen domain is SEQ ID NO:16.

40. A method of decreasing the level of BMP1 in a cell, said method comprising contacting a cell expressing BMP1 with a BMP1 inhibiting amount of collagen triple helix repeat containing 1 (CTHRC1), thereby decreasing the level of BMP1 in said cell.

41. A method of decreasing the level of BMP1 mRNA in a cell, said method comprising contacting a cell with a BMP1 mRNA expression-inhibiting amount of CTHRC1, thereby decreasing the level of BMP1 mRNA in said cell.

42. A method of increasing the level of BMP1 in a cell, said method comprising contacting a cell expressing BMP1 with a BMP1 increasing amount of a collagen triple helix repeat containing 1 (CTHRC1) inhibitor, thereby increasing the level of BMP1 in said cell.

43. A method of increasing the level of BMP1 mRNA in a cell, said method comprising contacting a cell with a BMP1 mRNA expression-increasing amount of a CTHRC1 inhibitor, thereby increasing the level of BMP1 mRNA in said cell.

44. A method of increasing the level of a propeptide in a cell, said method comprising contacting a cell with a BMP1 inhibiting amount of CTHRC1, thereby increasing the level of said propeptide in said cell.

45. The method of claim 5, wherein said propeptide is selected from the group consisting of a procollagen and a propeptide of lysyl-oxidase.

46. A method of inhibiting collagen formation by a cell, said method comprising contacting said cell with a BMP1 inhibiting amount of CTHRC1, thereby inhibiting collagen formation by said cell.

47. A method of decreasing bone matrix formation by a cell, said method comprising contacting a cell with a BMP1 inhibiting amount of CTHRC1, thereby decreasing bone matrix formation by said cell.

48. A method of decreasing the level of collagen in a cell, said method comprising contacting a cell with a BMP1 inhibiting amount of CTHRC1, thereby decreasing the level of collagen in said cell.

49. The method of claim 48, wherein said collagen is type I collagen.

50. A method of increasing the level of procollagen in a cell, said method comprising contacting a cell with a BMP1 inhibiting amount of CTHRC1, thereby increasing the level of procollagen in said cell.

51. A method of increasing the level of chordin in a cell, said method comprising contacting a cell with a BMP1 inhibiting amount of CTHRC1, thereby increasing the level of chordin in said cell.

52. A method of inhibiting cross-linking of collagen fibrils in a cell, said method comprising contacting a cell with a BMP1 inhibiting amount of CTHRC1, wherein BMP1 is responsible for processing a propeptide lysyl-oxidase, and further wherein said lysyl-oxidase mediates cross-linking said collagen fibrils, thereby inhibiting cross-linking of collagen fibrils in said cell.

53. A method of inhibiting plaque rupture in a blood vessel, said method comprising administering a collagen matrix production enhancing amount of a CTHRC1 inhibitor to a blood vessel comprising a plaque, thereby inhibiting plaque rupture in said blood vessel.

54. The method of claim 53, wherein said CTHRC1 inhibitor is selected from the group consisting of an antibody that specifically binds with CTHRC1 and a CTHRC1 antisense nucleic acid.

55. A method of identifying a compound that affects collagen production in a cell, said method comprising contacting a cell comprising CTHRC1 with a test compound and assessing the level of CTHRC1 in said cell, wherein a higher or lower level CTHRC1 in said cell contacted with said test compound compared with the level of CTHRC1 in a second otherwise identical cell not contacted with said test compound is an indication that said test compound inhibits collagen production in said cell, thereby identifying a compound that inhibits collagen production in said cell.

56. The method of claim 55, wherein said test compound affects the level of BMP1 in said cell.

57. The method of claim 55, wherein said test compound affects the level of BMP1 mRNA in said cell.

58. The method of claim 55, wherein said test compound affects the level of procollagen in said cell.

59. The method of claim 55, wherein said test compound affects the level of chordin in said cell.

60. A compound identified by the method of claim 55.

61. A method of decreasing collagen formation in a mammal in need thereof, said method comprising administering a BMP1 inhibiting amount of CTHRC1 to said mammal, whereby inhibiting BMP1 reduces collagen production, thereby decreasing collagen formation in said mammal.

62. The method of claim 61, wherein said mammal has a condition mediated by collagen formation and further wherein said condition is selected from the group consisting of wound scarring, wound healing, keloid formation, inflammation-associated scarring, pulmonary fibrosis, and angioplasty-associated vascular fibrosis.

63. A method of increasing bone matrix production in a cell, said method comprising administering an effective amount of an inhibitor of CTHRC1 to said cell, wherein inhibition of CTHRC1 increases the level of BMP1 in said cell, and further wherein increasing said level of BMP1 increases bone matrix production, thereby increasing bone matrix production in said cell.

64. A method of increasing collagen production in a mammal in need thereof, said method comprising administering an effective amount of an inhibitor of CTHRC1 to said mammal, wherein inhibition of CTHRC1 increases the level of BMP1 in said mammal, and further wherein increasing said level of BMP1 increases processing of fibrillar collagen, thereby increasing collagen production in said mammal.

65. A method of treating a disease mediated by expression of BMP1 in a mammal in need thereof, said method comprising administering to said mammal a BMP1 inhibiting amount of CTHRC1, thereby treating said disease mediated by expression of BMP1 in said mammal.

66. A kit for decreasing the level of BMP1 in a cell, said kit comprising a BMP1 inhibiting amount of collagen triple helix repeat containing 1 (CTHRC1), said kit further comprising an applicator, and an instructional material for the use thereof.

67. A kit for decreasing the level of BMP1 mRNA in a cell, said kit comprising a BMP1 inhibiting amount of collagen triple helix repeat containing 1 (CTHRC1), said kit further comprising an applicator, and an instructional material for the use thereof.

68. A kit for increasing the level of BMP1 in a cell, said kit comprising a BMP1 increasing amount of a collagen triple helix repeat containing 1 (CTHRC1 ) inhibitor, said kit further comprising an applicator, and an instructional material for the use thereof.

69. A kit for increasing the level of a propeptide in a cell, said kit comprising a BMP1 inhibiting amount of CTHRC1, said kit further comprising an applicator, and an instructional material for the use thereof.

70. The kit of claim 69, wherein said propeptide is selected from the group consisting of a procollagen and a propeptide of lysyl-oxidase.

71. A kit for inhibiting collagen formation by a cell, said kit comprising a BMP1 inhibiting amount of CTHRC1, said kit further comprising an applicator, and an instructional material for the use thereof.

72. A kit for decreasing bone matrix formation by a cell, said kit comprising a BMP1 inhibiting amount of CTHRC1, said kit further comprising an applicator, and an instructional material for the use thereof.

73. A kit for decreasing the level of collagen in a cell, said kit comprising a BMP1 inhibiting amount of CTHRC1, said kit further comprising an applicator, and an instructional material for the use thereof.

74. The kit of claim 73, wherein said collagen is type I collagen.

75. A method of increasing the level of bone morphogenetic protein 4 (BMP4) in a cell, said method comprising contacting a cell expressing BMP4 with a collagen triple helix repeat containing 1 (CTHRC1) in an amount sufficient to increase said level of said BMP4 in said cell, thereby increasing the level of BMP4 in said cell.

76. A method of increasing the level of BMP4 promoter activity in a cell, said method comprising contacting a cell with CTHRC1 in an amount sufficient to increase the level of said BMP4 promoter activity in said cell, thereby increasing the level of BMP4 promoter activity in said cell.

77. A method of promoting bone growth in a mammal, said method comprising contacting a mammal with CTHRC1 in an amount sufficient to increase the level of BMP4 in said mammal, thereby promoting bone growth in said mammal.

78. A method of promoting differentiation of a stem cell, said method comprising contacting said stem cell with CTHRC1 in an amount sufficient to increase the level of BMP4 in said stem cell, thereby promoting differentiation of said stem cell.

79. A method of decreasing the level of osteopontin (OPN) in a cell, said method comprising contacting said cell with CTHRC1 in an amount sufficient to increase the level of BMP4, thereby decreasing the level of OPN in said cell.

80. A method of increasing the level of OPN in a cell, said method comprising contacting said cell with a CTHRC1 inhibiting amount of a CTHRC1 inhibitor, thereby increasing the level of OPN in said cell.

81. A method of identifying a compound that effects a CTHRC1-mediated reduction of BMP4 in a cell, said method comprising contacting a CTHRC1-containing cell with a test compound, wherein a lower level of BMP4 in said cell contacted with said test compound compared with the level of BMP4 in a second otherwise identical cell not contacted with said test compound is an indication that said test compound reduces the level of BMP4 in said cell, and further wherein said test compound affects the activity of CTHRC1, thereby identifying a compound that effects a CTHRC1 -mediated reduction of BMP4 in said cell.

82. A method of treating a disease mediated by BMP4 in a mammal in need thereof, said method comprising administering to a mammal afflicted with a disease mediated by BMP4 a CTHR1 inhibiting amount of a CTHRC1 inhibitor, thereby treating said disease mediated by BMP4 in said mammal in need thereof.

83. A method of treating a disease mediated by under-expression of BMP4 in a mammal in need thereof, said method comprising administering to a mammal afflicted with said disease a BMP4 expression-inducing amount of CTHRC1.

84. A method of increasing the level of a muscle segment homeobox 1 (Msx1) in a cell, said method comprising contacting a cell expressing BMP4 with a collagen triple helix repeat containing 1 (CTHRC1) in an amount sufficient to increase said level of said BMP4 in said cell, wherein increasing the level of BMP4 mediates an increase in the level of Msx1, thereby increasing the level of Msx1 in said cell.

85. A kit for increasing the level of bone morphogenetic protein 4 (BMP4) in a cell, said kit comprising an amount of collagen triple helix repeat containing 1 (CTHRC1) sufficient to increase said level of said BMP4 in said cell, said kit further comprising an applicator, and an instructional material for the use thereof.
